(19) United States
(12) Patent Application Publication
(10) Pub. No.: US 2010/0016270 A1
(43) Pub. Date: Jan. 21, 2010

(54) BRIDGED POLYCYCLIC COMPOUND BASED COMPOSITIONS FOR CONTROLLING CHOLESTEROL LEVELS

(76) Inventor: Jeffery A. Whiteford, Belmont, CA (US)

Correspondence Address: MEYERTONS, HOOD, KIVLIN, KOWERT & GOETZEL, P.C., P.O. BOX 398, AUSTIN, TX, 78767-0398, US

(21) Appl. No.: 12/488,269

(22) Filed: Jun. 19, 2009

Related U.S. Application Data
(60) Provisional application No. 61/074,475, filed on Jun. 20, 2008.

(51) Int. Cl. A61K 31/395; C07D 487/00

(52) U.S. Cl. 514/183; 540/477

(57) ABSTRACT

A pharmaceutically active agent, a pharmaceutically active agent carrier and method of use thereof are described. In some embodiments, a system may include a composition. The composition may include one or more bridged polycyclic compounds. At least one of the bridged polycyclic compounds may include at least two cyclic groups, and at least two pharmaceutically active agents may be associated with the bridged polycyclic compound. In some embodiments, one or more bridged polycyclic compounds may be administered to a subject to improve cardiac and/or cardiovascular health. In some embodiments, one or more bridged polycyclic compounds may be administered to a subject to control cholesterol levels.

### PRIORITY CLAIM

This application claims priority to U.S. Provisional Patent Application No. 61/074,475 entitled “BRIDGED POLYCYCLIC COMPOUND BASED COMPOSITIONS FOR CONTROLLING CHOLESTEROL LEVELS” filed on Jun. 20, 2008, which is incorporated by reference herein.

### BACKGROUND OF THE INVENTION

1. Field of the Invention

The present disclosure generally relates to bridged polycyclic based compounds for the inhibition and amelioration of disease. More particularly, the disclosure generally relates to systems and methods for formulating antiviral, antibacterial, antifungal, antidisease compositions using these bridged polycyclic based compounds.

2. Description of the Relevant Art

Dendrimers are branched polymers with densely packed end-functional groups that can be used to attach the dendrimers to bioactive molecules such as drugs, targeting ligands and imaging agents. Since a significant portion of a dose of pharmaceutical drugs is lost in the circulation due to impaired uptake by the cells especially in the case of drug resistant cells. The actual concentration of a drug inside the cells is much less than what is present extracellularly. To increase the effectiveness of treatment of diseases it is important to increase the intracellular amount of the drug. Dendrimers have already been used as a carrier agent for several known antiviral agents. Attaching these known agents to a dendrimer has been shown to increase the activity of the agent verses using the agent alone and uncoupled to a dendrimer. However, there are problems associated with using dendrimers, especially when scaling up production to commercial quantities.

Two main methods exist for the synthesis of dendrimers: a divergent method (where the dendrimer is assembled in a totally linear manner) or a convergent method (where fragments of the dendrimer are condensed together). These two methods both suffer from major problems when it comes to practical synthesis, in particular, the necessity for repeated and time-consuming purifications.

Additional problems associated with the synthesis of dendrimers include: defects in the molecular structure; and the crowded molecular structure of dendrimers. The molecular structure of dendrimers is typically so crowded that many times other molecules become trapped within the spaces within the molecular structure of the dendrimer.

Therefore there is a need for a pharmaceutical composition comprising a compound which increases the intracellular availability of pharmaceutical drugs but which is easier and cheaper to synthesize than dendrimers and which are capable of more easily attaching different functionalities.

There are many maladies present in society (human and animal) which would benefit from a compound or composition capable of increasing intracellular concentrations of pharmaceutically active compounds wherein the carrier itself is substantially nonsystemic. A compound which is substantially nonsystemic may then pass through a subject's digestive track (e.g., for orally administered compounds) without being absorbed by into the bloodstream of the circulatory system. Advantages of nonsystemic compounds include, but are not limited to, decreased inadvertent and/or unforeseen adverse side effects, due mainly to the compounds limited exposure to the body as a whole. Typically high molecular weigh polymers are nonsystemic compounds which are not absorbed into the body of humans or animals and generally, if ingested, will pass through the digestive track and be expelled with excreted fecal matter or urine. It would be advantageous to have a nonsystemic polymeric or non polymeric compound capable of delivering pharmaceutically active drugs and/or functioning to absorb or sequester undesirable materials (and/or materials naturally found in the body which have accumulated in the body to unnaturally excessive levels) while passing through the body.

Cholesterol is eliminated from the body by converting cholesterol to bile acids. Bile acids are then excreted from the body as neutral steroids. Bile acids are synthesized from cholesterol in the liver and enter the bile as glycine and taurine conjugates. The conjugates are released in salt form in bile during digestion and act as detergents to solubilize and assist in digesting dietary fats. After digestion, bile acid salts are predomenantly reabsorbed in the ileum, complexed with proteins, and returned to the liver. Any bile acid salts not reabsorbed by active transport are excreted through the distal ileum and large intestine as feces.

Reabsorption of bile acids from the intestine conserves lipoprotein cholesterol in the bloodstream. Reducing reabsorption of bile acids within the intestinal tract can lower levels of bile acid leading to replacement of bile acids in the liver through synthesis from cholesterol. Reducing reabsorption of bile acids can therefore result in a lowering of circulating blood cholesterol levels.

One method of inhibiting bile acid reabsorption may include oral administration of compounds that sequester the bile acids within the intestinal tract inhibiting reabsorption of the bile acids. The sequestered bile acids may then be excreted from the body.

Orlistat (trademark XENICAL®) inhibits gastrointestinal lipases. Gastrointestinal lipases break down ingested fat. Inhibition of gastrointestinal lipase results in unabsorbed fat is excreted in the feces. Orlistat has been shown to inhibit fat absorption and to reduce body weight.

What is needed therefore is an easy to use, effective system for maintaining cardiovascular health. Preferably such methods and compositions should be easy-to-use and comprise antimicrobial agents. Such methods and compositions should provide long-term effectiveness against cholesterol level problems, including lipid and LDL levels. Such methods and compositions should provide long-term effectiveness against high cholesterol and/or weight problems. Such methods and compositions should be affordable, safe and easy to use on a regular basis.

### SUMMARY

Embodiments of the present invention address the problems described above by providing novel compositions and methods for altering cholesterol levels, altering triglyceride levels, reducing weight, and/or suppressing hunger. The present invention solves the problems described above by providing novel compositions and methods for reducing and/or ameliorating maladies associated with high LDL, cholesterol and or lipids in general. Embodiments of the present invention provide unique methods and compositions that are safe and effective for regular use by both humans and animals.

In some embodiments, a chemical composition may include a chemical compound. The chemical compound may include one or more bridged polycyclic compounds. At least one of the bridged polycyclic compounds may include at least two cyclic groups. At least two pharmaceutically active agents and/or derivatives of pharmaceutically active agents may be coupled to the bridged polycyclic compound.

In some embodiments, at least one of the pharmaceutically active agents may include an anti-inflammatory agent, an antimicrobial agent, a lipase inhibitor, a bile acid sequestrant, a hunger suppressant, triglyceride reduction agent, lipid reduction agent, weight reduction agent, and/or a cholesterol reduction agent.

In some embodiments, a chemical composition may include a chemical compound, wherein the chemical compound has a general structure (Ia):

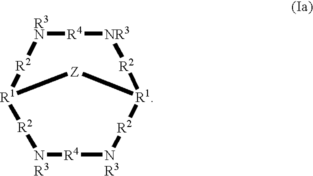

Each R¹ may be independently an alkyl group, a substituted alkyl group, an aryl group, a substituted aryl group, N, N⁺H, N⁺R³, a heterocycle group, or a substituted heterocycle group. Each R² may be independently an alkyl group, a substituted alkyl group, an aryl group, a substituted aryl group, a heterocycle group, a substituted heterocycle group, a covalent bond, or an alkene. Each R³ may be independently a hydrogen, pharmaceutically active agent, an ester, an alkyl group, a substituted alkyl group, an aryl group, a substituted aryl group, a heterocycle group, a substituted heterocycle group, an alkene, an ether, an ester, a PEG, an amide, an amine, a guanidine, or a PEI. Each R⁴ may be independently an alkyl group, a substituted alkyl group, an aryl group, a substituted aryl group, a heterocycle group, a substituted heterocycle group, an ether, an amide, an alcohol, an ester, a sulfonamide, a sulfanilamide, or an alkene. Z may include at least one bridge. At least one of the bridges may be —R²—N⁺R³₂—R⁴—N⁺R³₂—R²—, —R²—NR³—R⁴—N⁺R³₂—R²—, —R²—NR³—R⁴—NR³—R²—, or —R²—N═R⁴═N—R²—. Each bridge may independently couple R¹ to R¹. The chemical compound may include one or more negatively charged counter ions.

In some embodiments, a bridged polycyclic compound may include a salt of compound Ia.

In some embodiments, a chemical composition may include a chemical compound, wherein the chemical compound has a general structure:

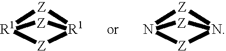

Z may include

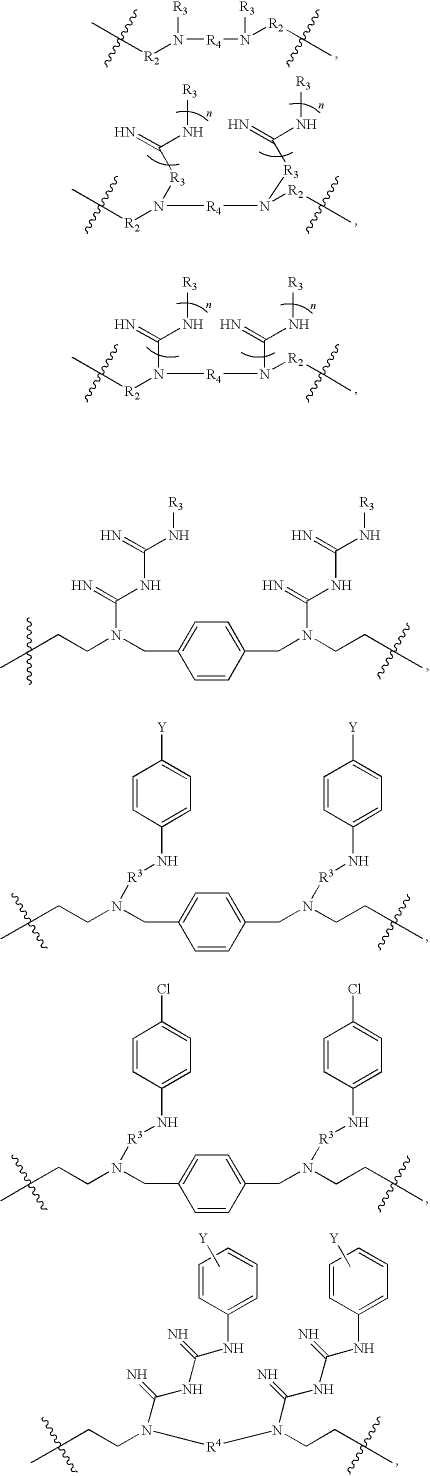

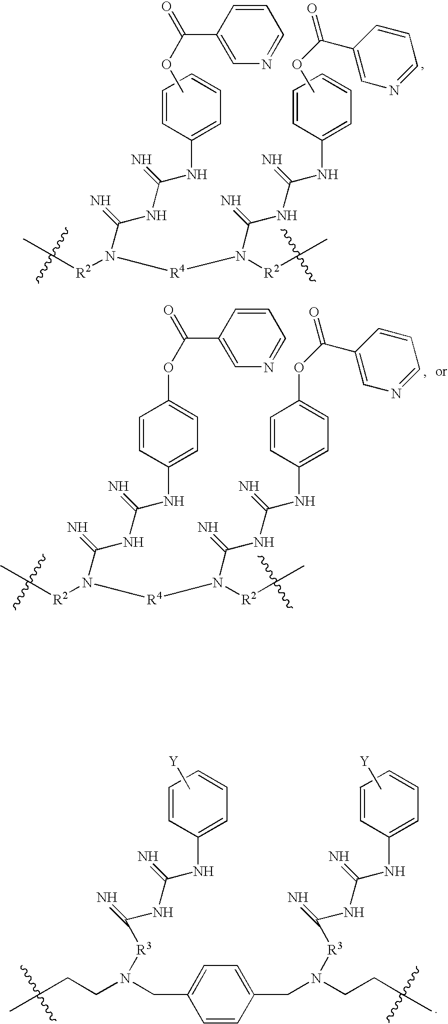

In some embodiments, Z may include at least two bridges.

In some embodiments, a chemical composition may include a chemical compound, wherein the chemical compound has a general structure:

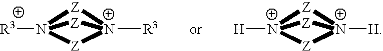

Z may include

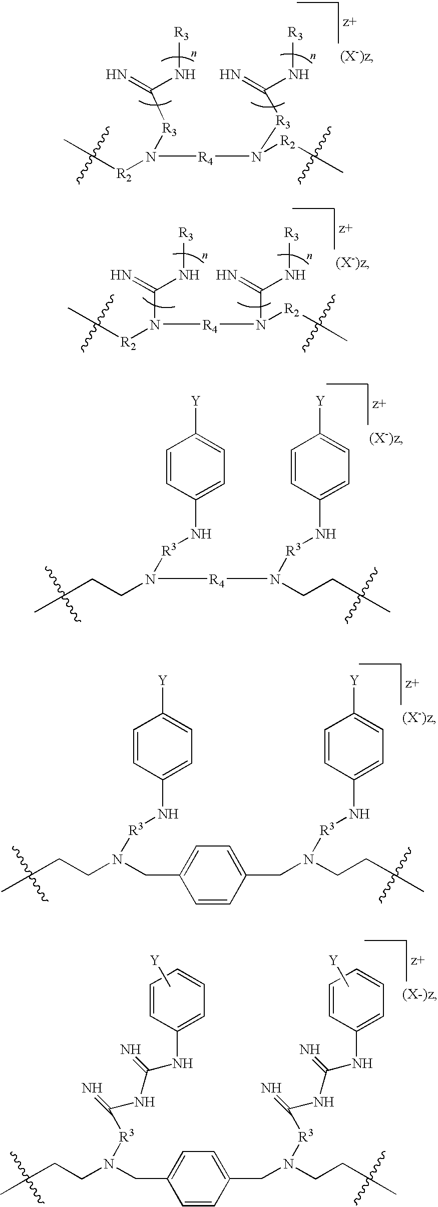

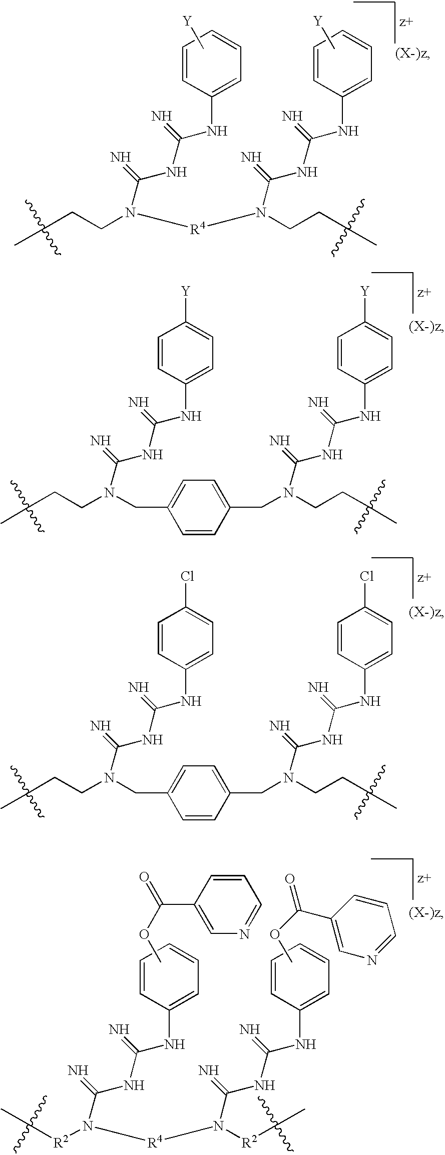

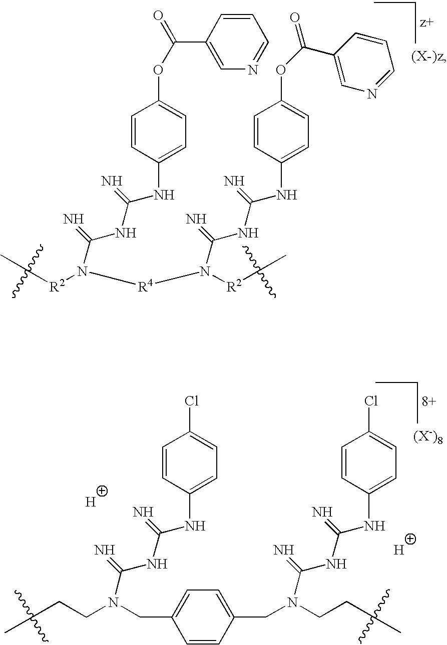

including combinations of Z, X and/or NaOAc,

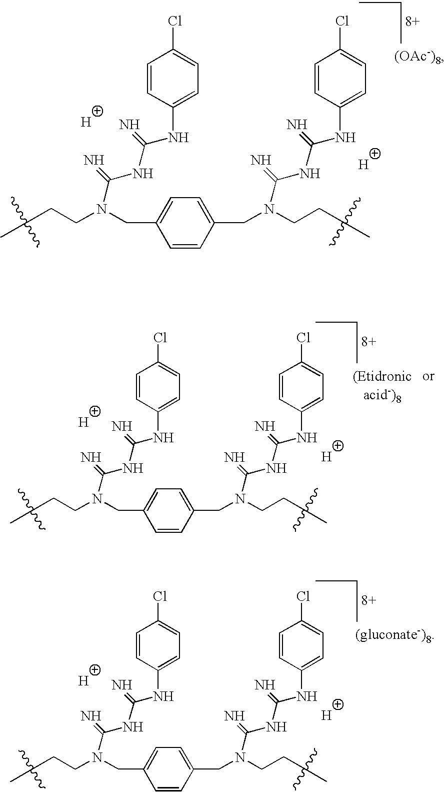

In some embodiments, R³ may include a guanidine moiety, a chlorhexidine derivative, an alcohol, a phenol, an ester, a phenol ester, a niacin ester, and/or a halogenated aryl moiety. R³ may include any of the other moieties associated with R³ herein.

In some embodiments, a chemical compound is a salt of the chemical compound. At least one counterion forming the salt may include an acetate ion.

In some embodiments, Y may include, for example, a halogen (e.g., Cl), an alcohol, an ester or a pharmaceutical active agent (e.g. nicotinic acid, nicotinic acid derivative). Y may include aryl, substituted aryl, alkyl, and/or substituted alkyl.

In some embodiments, X may include a counter ion. X may include a pharmaceutically active agent (e.g., Etidonate, Butyrate, Pinolenate, Hydroxycitrate).

In some embodiments, a chemical composition may include a polymer or a prepolymer. At least one polymer is poly(vinyl acetate-co-crotonic acid).

In some embodiments, a z may represent a charge on the chemical compound and an appropriate number of counterions. z may range from 1-16, 2-14, 6-14, 8-14, or 12-20.

In some embodiments, y may represent a number of bridges coupling the Nitrogens of the chemical compound. y may range from 3-8, 3-5, or 3-4.

In some embodiments, n may range from 1-8, 1-4, 2-4, or 1-3. n may be at least 2.

In some embodiments, a chemical composition may include at least one solvent.

In some embodiments, a chemical composition may include water and/or an alcohol (e.g., ethanol).

In some embodiments, a chemical composition may include a pharmaceutically acceptable viscous liquid (e.g., glycerin).

In some embodiments, a protective coating composition may include a compound. A compound may include a bridged polycyclic compound. A bridged polycyclic compound may be a cavitand. Portions of the bridged polycyclic compound may include two or more quaternary ammonium moieties. The coating composition may be antimicrobial.

In some embodiments, a protective coating composition may be antimicrobial.

In some embodiments, a compound may include a shape with a substantially curved surface.

In some embodiments, a coating may inhibit microbial adhesion.

In some embodiments, a compound may have a minimum inhibitory concentration of less than 0.1 mg/mL.

In some embodiments, a composition may have a minimum inhibitory concentration of less than 0.05 mg/mL.

In some embodiments, at least one R¹ is N⁺R³. In some embodiments, at least one R¹ is

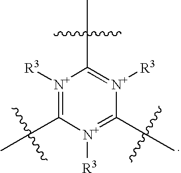

In some embodiments, at least one R³ is hydrophilic. In some embodiments, at least one R³ is a polymer. In some embodiments, at least one R³ is an oxazoline polymer. In some embodiments, at least one R³ is hydrophobic.

In some embodiments, at least one R⁴ may be

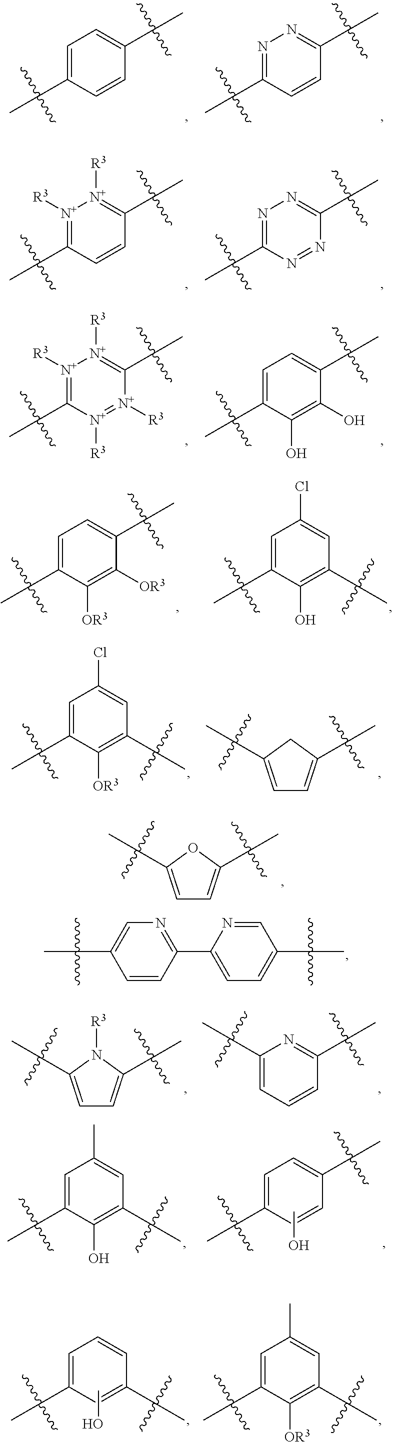

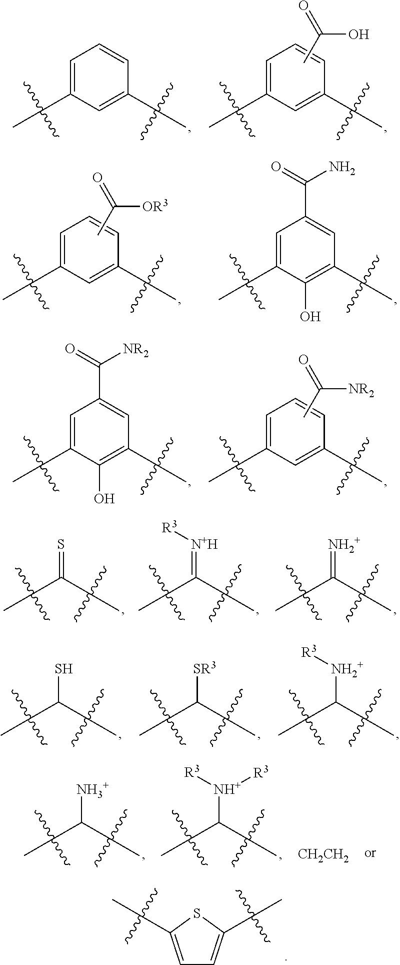

In some embodiments, a composition may include at least one metal (M) coordinated to at least a portion of the compound. At least one M may include a cation. At least one M may be positioned inside a space defined by R² and R⁴, and wherein at least one M is coordinated to one or more N+R³₂'s.

In some embodiments, at least one X may include a halogen ion.

In some embodiments, at least one X may include one or more elements with antimicrobial activity.

In some embodiments, at least one X may include one or more elements with anti- inflammatory activity

In some embodiments, at least one X may include boron.

In some embodiments, a composition may include one or more metals and/or metal ions with antimicrobial properties.

In some embodiments, a composition may include one or more metals and/or metal ions with anti-inflammatory properties.

In some embodiments, at least a portion of a chemical composition may form an antimicrobial coating over at least a portion of a surface. The chemical composition may include one or more bridged polycyclic compounds. At least one of the bridged polycyclic compounds may include at least two cyclic groups.

In some embodiments, a compound and/or a composition may have a minimum inhibitory concentration of greater than 900 μM (e.g., 900 μM-1500 μM, 900 μM -2000 μM, 1500 μM-2500 μM, etc.). In some embodiments, a compound and/or a coating composition may have a minimum inhibitory concentration of less than 10.0 mg/mL, less than 5.0 mg/mL, less than 1.0 mg/mL, less than 0.1 mg/mL, or less than 0.05 mg/mL. In such compositions, antimicrobial properties may not be the primary function of a coating composition.

The composition may be in the form of a gel, a foam, a sealant, a varnish, a resin, and/or a coating.

In some embodiments, a composition may include a coalescing solvent.

The method may include using the composition as a bonding agent.

The method may include using the composition as a resin cement.

The method may include using the composition as a sealant.

The method may include using the composition as a varnish.

The method may include using the composition as a resin.

In some embodiments, an oral surface may be coated with a coating. The coating may include a chemical composition at least a portion of which forms an antimicrobial coating over at least a portion of the oral surface. The coating may include a chemical composition at least a portion of which forms an anti-inflammatory coating over at least a portion of the oral surface. The coating may include a chemical composition at least a portion of which decreases bleeding over at least a portion of the oral surface. The coating may include a chemical composition at least a portion of which decreases inflammation over at least a portion of the oral surface.

In some embodiments, a composition may include bridged polycyclic compounds. Bridged polycyclic compounds may include cholesterol controlling (e.g., reducing) pharmaceutically active agents (e.g., nicotinic acid derivatives, niacin derivatives). The composition may be used to form a coating in at least a portion of an oral cavity. The coating may function to release pharmaceutically active agents slowly over time as the coating dissolves. As the coating dissolves the pharmaceutically active agents may be released reducing cholesterol. Slowly releasing certain pharmaceutically active agents may reduce some side effects (e.g., flushing associated with nicotinic acid). In some embodiments, slow release of pharmaceutically active agents may allow agents to be administered in a controlled manner for subjects (including humans and/or animals) who refuse to or for other reasons are not capable of taking their medicine in a more traditional manner.

In some embodiments, a chemically active derivative of a pharmaceutically active agent may be mixed with a coating composition including bridged polycyclic compounds. For example, Niacin may be converted into an acid chloride and mixed with polyvinyl acetate co-crotonic acid and one or more bridged polycyclic compounds. Such a composition when applied to a surface to form a coating may function as a slow release bile acid sequestrant (bridged polycyclic compound) LDL/triglyceride lowering (niacin derivative) drug formulation. A discussion of nicotinic acid and nicotinic acid derivatives (e.g., esters) and their use as cholesterol lowering preparations may be found in U.S. Pat. No. RE32,581 to Scherm et al. and U.S. Pat. No. 4,321,268 Scherm et al., which are incorporated by reference as if fully set forth herein.

In some embodiments, a method of inhibiting or ameliorating a disease may include administering to a subject an effective amount of a pharmaceutically acceptable formulation comprising a chemical composition as described herein.

In some embodiments, a subject may include a animal, mammal (e.g., canine, feline), and/or a human.

In some embodiments, a method may include administering the pharmaceutically acceptable formulation to a subject parenterally, intracoronary administration, subcutaneously, orally, and/or topically. Topical administration may be in the form of a gel and/or by self-administration of a topical formula.

In some embodiments, a method may include administering at least two different pharmaceutically active agents. The agents may be coupled to the same and/or different bridged polycyclic compounds.

In some embodiments, a chemical compound may decompose during use, wherein one or more of the products of the decomposition may be more biologically active relative to the chemical compound.

In some embodiments, a method may include administering the pharmaceutically acceptable formulation to a subject in the form of an emulsion.

### BRIEF DESCRIPTION OF THE DRAWINGS

Advantages of the present invention may become apparent to those skilled in the art with the benefit of the following detailed description of the preferred embodiments and upon reference to the accompanying drawings.

FIG. 1 depicts a graphical representation of time kill assay tests for a bridged polycyclic compound tested against Haemophilus Actinomycetemcomitans.

FIG. 2 depicts a graphical representation of time kill assay tests for a bridged polycyclic compound tested against Streptococcus mutans.

FIG. 3 depicts a graphical representation of time kill assay tests for a bridged polycyclic compound tested against Porphymonas Gingivalis.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DEFINITIONS

Unless defined otherwise, all technical and scientific terms used herein have the same meaning as commonly understood by one of ordinary skill in the art.

The term “accelerator” as used herein generally refers to a substance that speeds a chemical reaction.

The term “acyl” as used herein generally refers to a carbonyl substituent, —C(O)R, where R is alkyl or substituted alkyl, aryl, or substituted aryl, which may be called an alkanoyl substituent when R is alkyl.

The terms “administration,” “administering,” or the like, as used herein when used in the context of providing a pharmaceutical, cosmeceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

The term “aldehyde” as used herein generally refers to any of a class of organic compounds containing the group —CHO

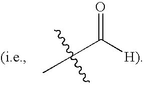

The term “aldehyde forming moiety” as used herein generally refers to any of a class of organic compounds which form an aldehyde in solution or react in an equivalent manner to an aldehyde such that an at least similar chemical product is achieved as would have been achieved with an aldehyde.

The terms “alkenyl” and “alkene” as used herein generally refer to any structure or moiety having the unsaturation C═C. As used herein, the term “alkynyl” generally refers to any structure or moiety having the unsaturation C≡C.

The term “alkoxy” generally refers to an —OR group, where R is an alkyl, substituted alkyl, aryl, substituted aryl. Alkoxy groups include, for example, methoxy, ethoxy, phenoxy, substituted phenoxy, benzyloxy, phenethyloxy, t-butoxy, and others.

The term “alkyl” as used herein generally refers to a chemical substituent containing the monovalent group CₙH₂ₙ, where n is an integer greater than zero. Alkyl includes a branched or unbranched monovalent hydrocarbon radical. An “n-mC” alkyl or “(nC-mC)alkyl” refers to all alkyl groups containing from n to m carbon atoms. For example, a 1-4C alkyl refers to a methyl, ethyl, propyl, or butyl group. All possible isomers of an indicated alkyl are also included. Thus, propyl includes isopropyl, butyl includes n-butyl, isobutyl and t-butyl, and so on. The term alkyl may include substituted alkyls.

The term “alkyl-aryl” as used herein generally refers to a chemical substituent containing an alkyl group or substituted alkyl group coupled to an aryl group or a substituted aryl group.

The term “altering,” as used herein, generally refers to a change in the magnitude of a biological parameter such as, for example, foci formation, tumorigenic or neoplastic potential, apoptosis, growth kinetics, expression of one or more genes or proteins of interest, metabolism, oxidative stress, replicative status, intercellular communication, or the like. “Alteration” may refer to a net increase or a net decrease in the biological parameter.

The term “amidine” as used herein generally refers to a specific derivative of oxoacids wherein the oxygens are replaced with nitrogens. An amidine may have a general structure of

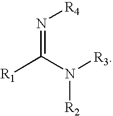

The terms “amino” or “amine” as used herein generally refer to a group —NRR′, where R and R′ may independently include, but are not limited to, hydrogen, alkyl, substituted alkyl, aryl, substituted aryl, alkyl-aryl, or acyl. Amine or amino may include a salt of the amine group.

The terms “amine forming moiety” as used herein generally refers to any of a class of organic compounds which form an amine in solution or react in an equivalent manner to an amine such that an at least similar chemical product is achieved as would have been achieved with an amine.

The terms “amphiphile” or “amphiphilic” as used herein generally refer to a molecule or species which exhibits both hydrophilic and lipophilic character. In general, an amphiphile contains a lipophilic moiety and a hydrophilic moiety. The terms “lipophilic” and “hydrophobic” are interchangeable as used herein. An amphiphile may form a Langmuir film.

Non-limiting examples of hydrophobic groups or moieties include lower alkyl groups, alkyl groups having 6, 7, 8, 9, 10, 11, 12, or more carbon atoms, including alkyl groups with 14-30, or 30 or more carbon atoms, substituted alkyl groups, alkenyl groups, alkynyl groups, aryl groups, substituted aryl groups, saturated or unsaturated cyclic hydrocarbons, heteroaryl, heteroarylalkyl, heterocyclic, and corresponding substituted groups. A hydrophobic group may contain some hydrophilic groups or substituents insofar as the hydrophobic character of the group is not outweighed. In further variations, a hydrophobic group may include substituted silicon atoms, and may include fluorine atoms. The hydrophobic moieties may be linear, branched, or cyclic.

Non-limiting examples of hydrophilic groups or moieties include hydroxyl, methoxy, phenyl, carboxylic acids and salts thereof, methyl, ethyl, and vinyl esters of carboxylic acids, amides, amino, cyano, isocyano, nitrile, ammonium salts, sulfonium salts, phosphonium salts, mono- and di-alkyl substituted amino groups, polypropyleneglycols, polyethylene glycols, glycosyl groups, sugars, epoxy groups, acrylates, sulfonamides, nitro, —OP(O)(OCH₂CH₂N⁺RRR)O⁻, guanidinium, aminate, acrylamide, pyridinium, piperidine, and combinations thereof, wherein each R is independently selected from H or alkyl. Further examples include polymethylene chains substituted with alcohol, carboxylate, acrylate, or methacrylate. Hydrophilic moieties may also include alkyl chains having internal amino or substituted amino groups, for example, internal —NH—, —NC(O)R—, or —NC(O)CH═CH₂— groups, wherein R is H or alkyl. Hydrophilic moieties may also include polycaprolactones, polycaprolactone diols, poly(acetic acid)s, poly(vinyl acetates)s, poly(2-vinyl pyridine)s, cellulose esters, cellulose hydroxylethers, poly(L-lysine hydrobromide)s, poly(itaconic acid)s, poly(maleic acid)s, poly(styrenesulfonic acid)s, poly(aniline)s, or poly(vinyl phosphonic acid)s. A hydrophilic group may contain some hydrophobic groups or substituents insofar as the hydrophilic character of the group is not outweighed.

The term “animal” as used herein generally refers to any member of the kingdom Animalia, comprising multicellular organisms that have a well-defined shape and usually limited growth, can move voluntarily, actively acquire food and digest it internally, and have sensory and nervous systems that allow them to respond rapidly to stimuli: some classification schemes also include protozoa and certain other single-celled eukaryotes that have motility and animallike nutritional modes. Generally the term animal as used herein does not refer to humans.

The term “antiinflammatory” as used herein generally refers to a substance acting to reduce certain signs of inflammation (e.g., swelling, tenderness, fever, and pain).

The term “antimicrobial” as used herein generally refers to a substance capable of destroying or inhibiting the growth of microbes, prevents the development of microbes, and/or inhibits the pathogenic action of microbes as well as viruses, fungi, and bacteria.

The term “aryl” as used herein generally refers to a chemical substituent containing an aromatic group (e.g., phenyl). An aromatic group may be a single aromatic ring or multiple aromatic rings which are fused together, coupled covalently, or coupled to a common group such as a methylene, ethylene, or carbonyl, and includes polynuclear ring structures. An aromatic ring or rings may include, but is not limited to, substituted or unsubstituted phenyl, naphthyl, biphenyl, diphenylmethyl, and benzophenone groups. The term “aryl” includes substituted aryls

The term “avian” as used herein generally refers to any of the biological family Aves including a class of vertebrates comprising the birds. Aves are generally characterized by have a complete double circulation, oviparous, reproduction, front limbs peculiarly modified as wings; and they bear feathers. All existing birds have a horny beak, without teeth.

The term “bridged polycyclic compound” as used herein generally refers to a compound that is composed of two or more cyclic systems that share two or more atoms. A cyclic system is formed from a group of atoms which together form a continuous loop. A bridged polycyclic compound may include a bridging atom or group of atoms that connects two or more non-adjacent positions of the same ring. An example of a bridged bicyclic system (i.e., a compound composed of two cyclic systems) with two atoms (atoms “A”) common to both cyclic systems is depicted below. One of the linking groups “L” represents a bridging atom or group of atoms.

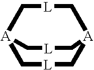

The term “canine” as used herein generally refers to any of the biological family Canidae including carnivorous mammals including wolves, jackals, foxes, coyote, and the domestic dog.

The term “cavitand” as used herein generally refers to a natural or synthetic molecular compound with enforced cavities large enough to complex complementary compounds or ions. More specifically, a cavitand may be generally defined as a three-dimensional compound that maintains a substantially rigid structure and binds a variety of molecules in the cavities produced by the structure of the three-dimensional compound.

The term “chelating agent or complexing agent” as used herein generally refers to any of various compounds that combine with metals to form chelates.

The term “coalescing agents or solvents” as used herein generally refers to any of various compounds that are used in coatings to promote film formation (e.g., in architectural and industrial latex coating).

The terms “coupling” and “coupled” with respect to molecular moieties or species, atoms, synthons, cyclic compounds, and nanoparticles refers to their attachment or association with other molecular moieties or species, atoms, synthons, cyclic compounds, and nanoparticles. The attachment or association may be specific or non-specific, reversible or non-reversible, the result of chemical reaction, or complexation or charge transfer. The bonds formed by a coupling reaction are often covalent bonds, or polar-covalent bonds, or mixed ionic-covalent bonds, and may sometimes be Coulombic forces, ionic or electrostatic forces or interactions.

The terms “crystalline” or “substantially crystalline”, when used with respect to nanostructures, refer to the fact that the nanostructures typically exhibit long-range ordering across one or more dimensions of the structure. It will be understood by one of skill in the art that the term “long range ordering” will depend on the absolute size of the specific nanostructures, as ordering for a single crystal typically does not extend beyond the boundaries of the crystal. In this case, “long-range ordering” will mean substantial order across at least the majority of the dimension of the nanostructure. In some instances, a nanostructure may bear an oxide or other coating, or may be comprised of a core and at least one shell. In such instances it will be appreciated that the oxide, shell(s), or other coating need not exhibit such ordering (e.g., it may be amorphous, polycrystalline, or otherwise). In such instances, the phrase “crystalline,” “substantially crystalline,” “substantially monocrystalline,” or “monocrystalline” refers to the central core of the nanostructure (excluding the coating layers or shells). The terms “crystalline” or “substantially crystalline” as used herein are intended to also encompass structures comprising various defects, stacking faults, atomic substitutions, etc., as long as the structure exhibits substantial long range ordering (e.g., order over at least about 80% of the length of at least one axis of the nanostructure or its core). It may be appreciated that the interface between a core and the outside of a nanostructure or between a core and an adjacent shell or between a shell and a second adjacent shell may contain non-crystalline regions and may even be amorphous. This does not prevent the nanostructure from being crystalline or substantially crystalline as defined herein.

The term “cyclic” as used herein generally refers to compounds having at least some of the atoms arranged in a ring or closed-chain structure.

The term “disease” as used herein generally refers to a disordered or incorrectly functioning organ, part, structure, or system of the body resulting from the effect of genetic or developmental errors, infection, poisons, nutritional deficiency or imbalance, toxicity, or unfavorable environmental factors; illness; sickness; ailment.

The terms “effective concentration” or “effective amount” as used herein generally refers to a sufficient amount of the pharmaceutically active agent is added to decrease, prevent or inhibit the growth of a virus and/or cancerous growth. The amount will vary for each compound and upon known factors related to the item or use to which the pharmaceutically active agent is applied.

The phrase “enteric coating” as used herein generally refers to a barrier applied to oral medication that controls the location in the digestive system where it is absorbed. Enteric refers to the small intestine, therefore enteric coatings prevent release of medication before it reaches the small intestine. Most enteric coatings work by presenting a surface that is stable at the highly acidic pH found in the stomach, but breaks down rapidly at a less acidic (relatively more basic) pH. For example, they will not dissolve in the acidic juices of the stomach (pH ˜3), but they will in the higher pH (above pH 5.5) environment present in the small intestine.

The term “feline” as used herein generally refers to any of the biological family Felidae including lithe-bodied carnivorous mammals (as the lion, lynx, and cheetah, as well as the common house cat). Felines having often strikingly patterned fur, comparatively short limbs with soft pads on the feet, usually sharp curved retractile claws, a broad and somewhat rounded head with short but powerful jaws equipped with teeth suited to grasping, tearing, and shearing through flesh, erect ears, and typically eyes with narrow or elliptical pupils and especially adapted for seeing in dim light.

The terms “functionalized” or “functional group” as used herein generally refers to the presence of a reactive chemical moiety or functionality. A functional group may include, but is not limited to, chemical groups, biochemical groups, organic groups, inorganic groups, organometallic groups, aryl groups, heteroaryl groups, cyclic hydrocarbon groups, amino (—NH₂), hydroxyl (—OH), cyano (—C≡N), nitro (NO₂), carboxyl (—COOH), formyl (—CHO), keto (—CH₂C(O)CH₂—), ether (—CH₂—O—CH₂—), thioether (—CH₂—S—CH₂—), alkenyl (—C═C—), alkynyl, (—C≡C—), epoxy

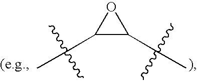

metalloids (functionality containing Si and/or B) and halo (F, Cl, Br, and I) groups. In some embodiments, the functional group is an organic group.

The term “gram-negative bacteria” or “gram-negative bacterium” as used herein generally refers to bacteria which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein generally refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

The term “guanidine” as used herein generally refers to

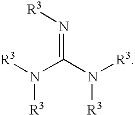

Guanidine may also refer to derivatives of guanidine

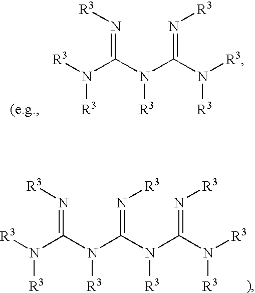

including, for example, salts of guanidine.

The term “heteroaryl” generally refers to an unsaturated heterocycle.

The term “heterocycle” as used herein generally refers to a closed-ring structure, in which one or more of the atoms in the ring is an element other than carbon. Heterocycle may include aromatic compounds or non-aromatic compounds. Heterocycles may include rings such as thiophene, pyridine, isoxazole, phthalimide, pyrazole, indole, furan, or benzo-fused analogues of these rings. Examples of heterocycles include tetrahydrofuran, morpholine, piperidine, pyrrolidine, and others. In some embodiments, “heterocycle” is intended to mean a stable 5- to 7-membered monocyclic or bicyclic or 7- to 10-membered bicyclic heterocyclic ring which is either saturated or unsaturated, and which consists of carbon atoms and from 1 to 4 heteroatoms (e.g., N, O, and S) and wherein the nitrogen and sulfur heteroatoms may optionally be oxidized, and the nitrogen may optionally be quaternized, and including any bicyclic group in which any of the above-defined heterocyclic rings is fused to a benzene ring. In some embodiments, heterocycles may include cyclic rings including boron atoms. The heterocyclic ring may be attached to its pendant group at any heteroatom or carbon atom which results in a stable structure. The heterocyclic rings described herein may be substituted on carbon or on a nitrogen atom if the resulting compound is stable. Examples of such heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzofuranyl, benzothiophenyl, carbazole, chromanyl, chromenyl, cinnolinyl, decahydroquinolinyl, furanyl, furazanyl, imidazolidinyl, imidazolinyl, imidazolyl, indolinyl, indolizinyl, indolyl, isobenzofuranyl, isochromanyl, isoindolinyl, isoindolyl, isoquinolinyl (benzimidazolyl), isothiazolyl, isoxazolyl, morpholinyl, naphthyridinyl, octahydroisoquinolinyl, oxazolidinyl, oxazolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, pyrrolyl, quinazolinyl, quinolinyl, quinoxalinyl, quinuclidinyl, carbolinyl, tetrahydrofuranyl, tetrahydroisoquinolinyl, tetrahydroquinolinyl, tetrazolyl, thianthrenyl, thiazolyl, thienyl, thiophenyl, triazinyl, xanthenyl. Also included are fused ring and spiro compounds containing, for example, the above heterocycles.

The term “initiator” as used herein generally refers to a substance that initiates a chemical reaction.

The term “ion” as used herein generally refers to an atom(s), radical, or molecule(s) that has lost or gained one or more electrons and has thus acquired an electric charge.

The terms “in need of treatment” or “in need thereof” when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

The term “malady” as used herein generally refers to any disorder or disease of the body or any undesirable or disordered condition including, but not limited to, illness, sickness, affliction, complaint, ailment, indisposition, virus, disease, fungus, infection, disease, etc.

The term “mammal” as used herein generally refers to any vertebrate of the class Mammalia. Mammalians generally having the body more or less covered with hair, nourishing the young with milk from the mammary glands, and, with the exception of the egg-laying monotremes, giving birth to live young. Generally the term mammal as used herein does not refer to humans.

The term “matrix” generally refers to a material, often a polymeric material and/or a prepolymeric material, into which a second material (e.g., a nanostructure) is embedded, surrounded, or otherwise associated. A matrix is typically composed of one or more monomers, but may include other matrix components/constituents. Often the matrix constituents include one or more “addressable” components or complementary binding pairs, that optionally promote assembly and/or cross-linkage of the matrix.

The term “medical device” as used herein generally refers to a device used which pertains to treating or determining the state of one's health. Medical devices are any article that contacts subjects or are used in health care, and may be for use either internally or externally.

The term “microbe” as used herein generally refers to a minute life form; a microorganism. In some embodiments, a microbe may include a bacterium that causes disease.

The term “monocrystalline” when used with respect to a nanostructure indicates that the nanostructure is substantially crystalline and comprises substantially a single crystal. When used with respect to a nanostructure heterostructure comprising a core and one or more shells, “monocrystalline” indicates that the core is substantially crystalline and comprises substantially a single crystal.

The terms “monofunctional”, “bifunctional”, “trifunctional”, and “multifunctional” generally refers to a number of attachment sites a particular compound, molecule, atom, etc. may include (monofunctional having one site, bifunctional having two sites, trifunctional having three sites, and multifunctional having more than one site).

The term “nanocrystal” as used herein generally refers to a nanostructure that is substantially monocrystalline. A nanocrystal thus has at least one region or characteristic dimension with a dimension of less than about 500 nm, e.g., less than about 200 nm, less than about 100 nm, less than about 50 nm, or even less than about 20 nm. The region or characteristic dimension may be along the smallest axis of the structure. Examples of such structures include nanowires, nanorods, nanotubes, branched nanowires, nanotetrapods, nanotripods, nanobipods, nanocrystals, nanodots, quantum dots, nanoparticles, nanoribbons, etc. Nanostructures may be substantially homogeneous in material properties, or in certain embodiments may be heterogeneous (e.g., heterostructures). Optionally, a nanocrystal may comprise one or more surface ligands (e.g., surfactants). The nanocrystal is optionally substantially single crystal in structure (a “single crystal nanostructure” or a “monocrystalline nanostructure”). Nanostructures may be fabricated from essentially any convenient material or material, the nanostructure may be prepared from an inorganic material, e.g., an inorganic conductive or semiconductive material. A conductive or semi-conductive nanostructure often displays 1-dimensional quantum confinement, e.g., an electron may often travel along only one dimension of the structure. Nanocrystals may be substantially homogeneous in material properties, or in certain embodiments may be heterogeneous (e.g., heterostructures). The term “nanocrystal” is intended to encompass substantially monocrystalline nanostructures comprising various defects, stacking faults, atomic substitutions, etc., as well as substantially monocrystalline nanostructures without such defects, faults, or substitutions. In the case of nanocrystal heterostructures comprising a core and one or more shells, the core of the nanocrystal is typically substantially monocrystalline, but the shell(s) need not be. The nanocrystals may be fabricated from essentially any convenient material or materials.

The terms “nanostructure” or “nanoparticle” are used herein to generally refer to a structure having at least one region or characteristic dimension with a dimension of less than about 500 nm, e.g., less than about 200 nm, less than about 100 nm, less than about 50 nm, or even less than about 20 nm. The region or characteristic dimension may be along the smallest axis of the structure. Examples of such structures include nanowires, nanorods, nanotubes, branched nanocrystals, nanotetrapods, tripods, bipods, nanocrystals, nanodots, quantum dots, nanoparticles, branched tetrapods (e.g., inorganic dendrimers), etc. Nanostructures may be substantially homogeneous in material properties, or in certain embodiments may be heterogeneous (e.g., heterostructures). Nanostructures may be, e.g., substantially crystalline, substantially monocrystalline, polycrystalline, amorphous, or a combination thereof. In one aspect, each of the three dimensions of the nanostructure has a dimension of less than about 500 nm, e.g., less than about 200 nm, less than about 100 nm, less than about 50 nm, or even less than about 20 nm. Nanostructures may comprise one or more surface ligands (e.g., surfactants).

The term “nonsystemic ” as used herein, generally refers to a compound or composition which is not substantially absorbable into the bloodstream of a human or animal.

The terms “oligomeric” and “polymeric” as used herein are generally used interchangeably herein to generally refer to multimeric structures having more than one component monomer or subunit.

The term “organ” is used herein to generally refer to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

The term “opthalmic” as used herein generally is of or relating to or resembling the eye; “ocular muscles”; “an ocular organ”; “ocular diseases”.

The term “oral surface” as used herein generally refers to a portion of the mouth and/or something positioned in and/or coupled to a portion of the mouth. For example an oral surface may include, but is not limited to, at least a portion of a tooth, at least a portion of the gum, at least a portion of the tongue, at least a portion of a dental fixture (e.g., a filling, a bridge, a cap a false tooth).

The term “otic” as used herein generally is of, relating to, or located near the ear; auricular.

The term “pharmaceutically acceptable salts” as used herein generally includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

The term “pharmaceutically active agent” as used herein generally refers to a drug or other substance that has therapeutic value to a living organism including without limitation antithrombotics, anticoagulants, antiplatelet agents, thrombolytics, antiproliferatives, antiviral, antitumor, anticancer, antimicrobial, antifungal, anti-inflammatories, agents that inhibit restenosis, smooth muscle cell inhibitors, antibiotics, and the like, and mixtures thereof.

Terms such as “pharmaceutical composition,” “pharmaceutical formulation,” “pharmaceutical preparation,” or the like, are used herein to generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release,” “controlled release,” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, included within the meaning of the term are pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

A “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

The term “pharmacologically inert,” as used herein, generally refers to a compound, additive, binder, vehicle, and the like, that is substantially free of any pharmacologic or “drug-like” activity.

The term “polycyclic,” as used herein, generally refers to a chemical compound having two or more atomic rings in a molecule. Steroids are polycyclic compounds.

The term “polymerizable compound,” as used herein, generally refers to a chemical compound, substituent or moiety capable of undergoing a self-polymerization and/or co-polymerization reaction (e.g., vinyl derivatives, butadienes, trienes, tetraenes, dialkenes, acetylenes, diacetylenes, styrene derivatives).

By “prophylactically effective amount” is meant an amount of a pharmaceutical composition that will substantially prevent, delay or reduce the risk of occurrence of the biological or physiological event in a cell, a tissue, a system, animal or human that is being sought by a researcher, veterinarian, physician or other caregiver.

The term “quaternary ammonium moiety,” as used herein, generally refers to a tetravalent charged nitrogen (e.g., N⁺R³₄).

The terms “Rⁿ” in a chemical formula refer to a hydrogen or a functional group, each independently selected, unless stated otherwise. In some embodiments the functional group may be an organic group. In some embodiments the functional group may be an alkyl group. In some embodiment, the functional group may be a hydrophobic or hydrophilic group.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

The term “subject” as used herein generally refers to a mammal (e.g., felines, canines), and in particular to a human.

The term “sealant,” as used herein, generally refers to any of various liquids, paints, chemicals, or soft substances that may be applied to a surface or circulated through a system of pipes or the like, drying to form a hard, substantially watertight coating. When used in the context of dentistry sealant generally refers to any of several transparent synthetic resins applied to the chewing surfaces of an oral cavity as a preventive measure against tooth decay in the occlusal pits and fissures.

The term “statin,” as used herein, generally refers to any of a class of lipid-lowering drugs that reduce serum cholesterol levels by, for example, inhibiting a key enzyme involved in the biosynthesis of cholesterol.

The term “substituted alkyl” as used herein generally refers to an alkyl group with an additional group or groups attached to any carbon of the alkyl group. Substituent groups may include one or more functional groups such as amino, quaternary ammonium moieties, and/or guanidine. Substituent groups may include one or more functional groups such as alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbons, heterocycles, and other organic groups.

The term “substituted alkyl-aryl” as used herein generally refers to an alkyl-aryl group with an additional group or groups attached to any carbon of the alkyl-aryl group. Additional groups may include one or more functional groups such as amino, quaternary ammonium moieties, and/or guanidine. Additional groups may include one or more functional groups such as lower alkyl, aryl, acyl, halogen, alkylhalo, hydroxy, amino, alkoxy, alkylamino, acylamino, acyloxy, aryloxy, aryloxyalkyl, thioether, heterocycles, both saturated and unsaturated cyclic hydrocarbons which are fused to the aromatic ring(s), coupled covalently or coupled to a common group such as a methylene or ethylene group, or a carbonyl coupling group such as in cyclohexyl phenyl ketone, and others.

The term “substituted aryl” as used herein generally refers to an aryl group with an additional group or groups attached to any carbon of the aryl group. Additional groups may include one or more functional groups such as amino, quaternary ammonium moieties, and/or guanidine. Additional groups may include one or more functional groups such as lower alkyl, aryl, acyl, halogen, alkylhalo, hydroxy, amino, alkoxy, alkylamino, acylamino, acyloxy, aryloxy, aryloxyalkyl, thioether, heterocycles, both saturated and unsaturated cyclic hydrocarbons which are fused to the aromatic ring(s), coupled covalently or coupled to a common group such as a methylene or ethylene group, or a carbonyl coupling group such as in cyclohexyl phenyl ketone, and others.

The term “substituted heterocycle” as used herein generally refers to a heterocyclic group with an additional group or groups attached to any element of the heterocyclic group. Additional groups may include one or more functional groups such as amino, quaternary ammonium moieties, and/or guanidine. Additional groups may include one or more functional groups such as lower alkyl, aryl, acyl, halogen, alkylhalos, hydroxy, amino, alkoxy, alkylamino, acylamino, acyloxy, aryloxy, aryloxyalkyl, thioether, heterocycles, both saturated and unsaturated cyclic hydrocarbons which are fused to the heterocyclic ring(s), coupled covalently or coupled to a common group such as a methylene or ethylene group, or a carbonyl coupling group such as in cyclohexyl phenyl ketone, and others.

The term “substrate” as used herein generally refers to a body or base layer or material (e.g., onto which other layers are deposited).

The phrase “therapeutically effective amount” generally refers to an amount of a drug or pharmaceutical composition that will elicit at least one desired biological or physiological response of a cell, a tissue, a system, animal or human that is being sought by a researcher, veterinarian, physician or other caregiver.

The term “thioether” as used herein generally refers to the general structure R—S—R′ in which R and R′ are the same or different and may be alkyl, aryl or heterocyclic groups. The group —SH may also be referred to as “sulfhydryl” or “thiol” or “mercapto.”

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

The term “topical” as used herein generally is of, pertaining to, or applied externally to a particular part of the body.

The term “virus” as used herein generally refers to an ultramicroscopic (20 to 300 nm in diameter), metabolically inert, infectious agent that replicates only within the cells of living hosts, mainly bacteria, plants, and animals: composed of an RNA or DNA core, a protein coat, and, in more complex types, a surrounding envelope.

### Bridged Polycyclic Compounds

New antimicrobials are required to combat the new antimicrobial resistant microbes. New antimicrobials may be effective verses microbes which are currently resistant to currently known antimicrobials. New antimicrobials may resist leaching off into the environment beyond a predetermined amount to inhibit polluting the environment unnecessarily (which may concurrently increase the occurrence of antimicrobial resistant microbes from overexposure of antimicrobials).

One strategy for combating antimicrobial resistant organisms is by modifying known antimicrobials to increase their effectiveness. In some embodiments, quaternary ammonium compounds may be modified to increase their effectiveness. It is typically thought that quaternary ammonium compounds denature the proteins of the bacterial or fungal cell, affect the metabolic reactions of the cell and allow vital substances to leak out of the cell, finally causing death. In addition, quaternary ammonium compounds are not known to be toxic towards higher forms of life (e.g., humans).

One of the main considerations in examining the mode of action is the characterization of quaternary ammonium compounds as cationic surfactants. This class of chemical reduces the surface tension at interfaces, and is attracted to negatively charged surfaces, including microorganisms. Quaternary ammonium compounds denature the proteins of the bacterial or fungal cell, affect the metabolic reactions of the cell and allow vital substances to leak out of the cell, finally causing death.

Most uses of quaternary ammonium compounds as antimicrobials involve formulations of disinfectants and sanitizers which are not bound to a surface, resulting in effluent stream pollution and contamination. They are simply wetted onto the surface such as in disinfecting wipes which are primarily ammonium salts as their liquid active ingredient. When they are incorporated into surfaces they are not crosslinked but are allowed to float to the surface thereby becoming depleted over time the same way silver and triclosan are incorporated in plastics. Coupling quaternary ammonium compounds to a surface or formation within a polymer matrix may inherently reduce the effectiveness of the quaternary ammonium compounds, by decreasing the accessibility of microbes to the most active cationic portion of the molecule. Increasing accessibility to the quaternary ammonium compounds within a surface coating or with any use increases the effectiveness of the quaternary ammonium compound.

In some embodiments, the effectiveness of an antimicrobial (e.g., quaternary ammonium compound) may be increased by coupling the antimicrobial within or on a curved surface, where the curved surface is on a molecular scale. For example, a curved surface may be created using nanoparticles (e.g., spherical nanoparticles). Nanoparticles may incorporate into their structure antimicrobial compounds with greater exposed surface area due to the curved surface of the nanoparticle.

In some embodiments, a compound may include a nanoparticle. The nanoparticle may include a bridged polycyclic compound. A compound may be formed using self-assembly techniques and principles. A compound may be formed from portions which are themselves antimicrobial (e.g., quaternary ammonium compounds). A compound may bind moieties to at least portions of itself which have, for example, antimicrobial properties.

In some embodiments, a protective coating composition may include a compound. A compound may be a bridged polycyclic compound. A bridged polycyclic compound may be a cavitand. Portions of the bridged polycyclic compound may include two or more quaternary ammonium moieties. The protective coating composition may be antimicrobial.

New carrier agents are required to more effectively deliver existing and future pharmaceutical agents.

One strategy for more effectively delivering pharmaceutical agents is to couple a multitude of pharmaceutical agents (e.g., a single type of agent or a combination of different agents) to a single molecular entity.

In some embodiments, the effectiveness of a pharmaceutically active agent may be increased by coupling the agent within or on a curved surface, where the curved surface is on a molecular scale. For example, a curved surface may be created using nanoparticles (e.g., spherical nanoparticles). Nanoparticles may incorporate into their structure pharmaceutically active agent with greater exposed surface area due to the curved surface of the nanoparticle.

In some embodiments, a pharmaceutically active agent may include using derivatives of pharmaceutically active agents. Pharmaceutically active agents may be modified in order to couple the agent to one or more bridged polycyclic compounds. Pharmaceutically active agents may be modified in order to increase their effectiveness.

In some embodiments, a compound may include a nanoparticle. The nanoparticle may include a bridged polycyclic compound. A compound may be formed using self-assembly techniques and principles. A compound may be formed from portions which are pharmaceutically active agents. A compound may bind moieties to at least portions of itself which are pharmaceutically active agents.

In some embodiments, a composition may include one or more bridged polycyclic compounds. At least one of the bridged polycyclic compounds may include at least two cyclic groups. A general example of a bridged polycylic compound including only two cyclic groups may include, but is not limited to, a compound 100 having a general structure

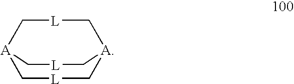

In some embodiments, a bridged polycylic compound may include two or more substituent groups coupled to the compound (e.g., compound 100). Substituents may include one or more functional groups such as amino, quaternary ammonium moieties, and/or guanidine. In some embodiments, at least two cyclic groups may be defined in part by quaternary ammonium moieties, by the nitrogen of the quaternary ammonium moiety comprising one of the atoms which forms a part of the cyclic structure itself. For example, a cyclic structure which is formed at least in part by a quaternary ammonium moiety may include, but is not limited to structure 101

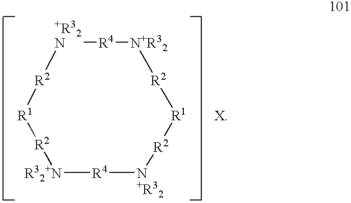

Structure 101 is an example of quaternary ammonium moieties defining at least in part a cyclic group, however, compound 101 is not an example of a polycyclic compound and compound 101 is not an example of a bridged polycyclic compound.

In some embodiments, a bridged polycyclic compound may include at least two quaternary ammonium moieties, at least three quaternary ammonium moieties, at least four quaternary ammonium moieties, at least five quaternary ammonium moieties, at least six quaternary ammonium moieties, at least seven quaternary ammonium moieties, or at least eight quaternary ammonium moieties.

In some embodiments, a compound 100 may have a general structure

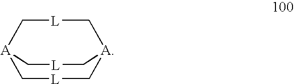

Compound 100 may be formed by coupling a trifunctional corner unit A with a bifunctional linker unit L as depicted in Scheme 2.

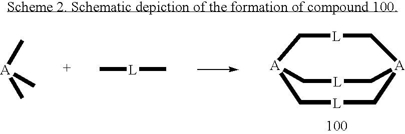

Scheme 2 should not be used to limit the disclosure set forth herein. Corner unit A may include multiple dentate linkers other than the one depicted in Scheme 2 (e.g., a trifunctional linker A is depicted in Scheme 2) including, but not limited to, bifunctional, tetrafunctional (e.g., compound 100a) etc. In some embodiments, a corner unit A may be coupled to a linker unit L in any multitude of ways known to one skilled in the art.

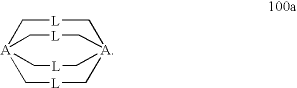

In some embodiments, a compound 100c may have a general structure

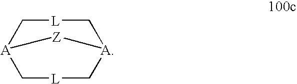

Compound 100c may be a bridged polycyclic compound. In some embodiments, Z may include at least one bridge. Bridge Z may couple 2 non adjacent atoms.

In some embodiments, at least one of the bridges is —R²—N⁺R³₂—R⁴—N⁺R³₂—R²—, such that each bridge independently couples A to A. In some embodiments, at least one of the bridges may be —R²—NR³—R⁴—N⁺R³₂—R²—. Each bridge may independently couple A to A. In some embodiments, at least one of the bridges may be —R²—NR³—R⁴—NR³—R²—. Each bridge may independently couple A to A. In some embodiments, at least one of the bridges may be —R²—N═R⁴═N—R²—. Each bridge may independently couple A to A.

For example when Z is 1 compound 100c may be a compound 100 having a general structure

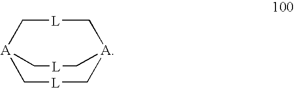

When, for example, Z is 2 a compound 100c may be a compound 100a having a general structure

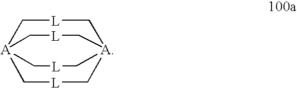

When, for example, Z is 3 a compound 100c may be a compound 100d having a general structure

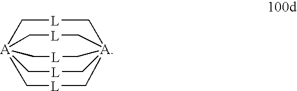

In some embodiments, a compound may include a bridged polycyclic compound formed from two corner units (e.g., compound 100b). Compound 100b may be formed by coupling a multifunctional (e.g., trifunctional) corner unit A with a second multifunctional (e.g., trifunctional) corner unit A as depicted in Scheme 2a.

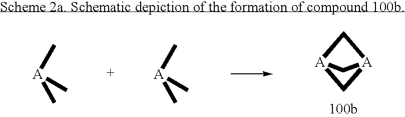

In some embodiments, a compound 102 may have a general structure

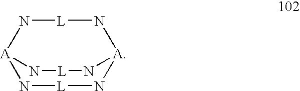

Compound 102 may include a moiety coupling corner unit A with linker unit L, the moiety including a nitrogen.

In some embodiments, a compound 103 may have a general structure

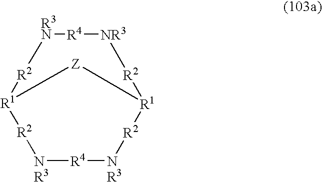

In some embodiments, R¹ may be independently alkyl, substituted alkyl, aryl, substituted aryl, N, N⁺R³, heterocycle, or substituted heterocycle. R² may be independently alkyl, substituted alkyl, aryl, substituted aryl, heterocycle, substituted heterocycle, covalent bond, or alkene. R³ may be independently a pharmaceutically active agent, alkyl, substituted alkyl, aryl, substituted aryl, heterocycle, substituted heterocycle, alkene, ether, PEG, contains boron, or PEI. In some embodiments, R³ may include one or more pharmaceutically active agents. Pharmaceutically active agents may include functional groups such as amino, quaternary ammonium moieties, and/or guanidine. Such functional groups may be coupled to or part of an alkyl-aryl, substituted alkyl, and/or substituted aryl. For example, an amino group may be coupled to a substituted aryl group or coupled to a substituted alkyl group. R⁴ may be independently alkyl, substituted alkyl, aryl, substituted aryl, N⁺R³, heterocycle, substituted heterocycle, alkyl ether, PEG, PEI, ether, or alkene. R⁴ may independently include amide, alcohol, ester, sulfonamide, or sulfanilamide. R⁴ may be independently alkyl, substituted alkyl, aryl, substituted aryl, heterocycle, substituted heterocycle, ether, amide, alcohol, ester, sulfonamide, sulfanilamide, or alkene. Z may include at least one bridge.

In some embodiments, at least one of the bridges may be —R²—N⁺R³₂—R⁴—N⁺R³₂—R²—. Each bridge may independently couple R¹ to R¹. In some embodiments, at least one of the bridges may be —R²—NR³—R⁴—N⁺R³₂—R²—. Each bridge may independently couple R¹ to R¹ . In some embodiments, at least one of the bridges may be —R²—NR³—R⁴—NR³—R²—. Each bridge may independently couple R¹ to R¹. In some embodiments, at least one of the bridges may be —R²—N═R⁴═N—R²—. Each bridge may independently couple R¹ to R¹.

For example when Z is 1 compound 103a may be a compound 104b having a general structure

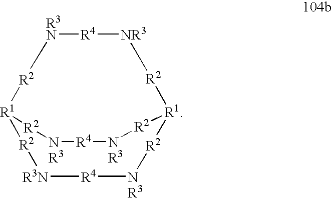

When, for example, Z is 2 a compound 103a may be a compound 104c having a general structure

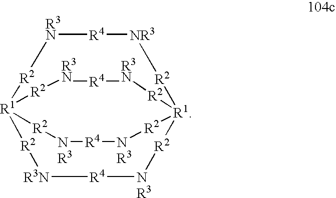

In some embodiments, a pharmaceutically active agent may include guanidine or a guanidine derivative. A known guanidine is Chlorhexidine. Chlorhexidine is a chemical antiseptic. Chlorhexidine functions as a bactericidal to both gram-positive and gram-negative microbes. It is considered less effective with some gram-negative microbes. Chlorhexidine is considered bacteriostatic. The mechanism of action is believed to be membrane disruption, in a similar manner to the quaternary ammonium salts discussed herein. Chlorhexidine may have a structure

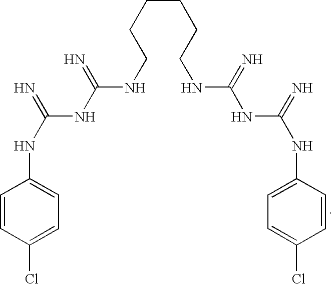

In some embodiments, a guanidine derivative may include a moiety having a structure (including a salt of the moiety)

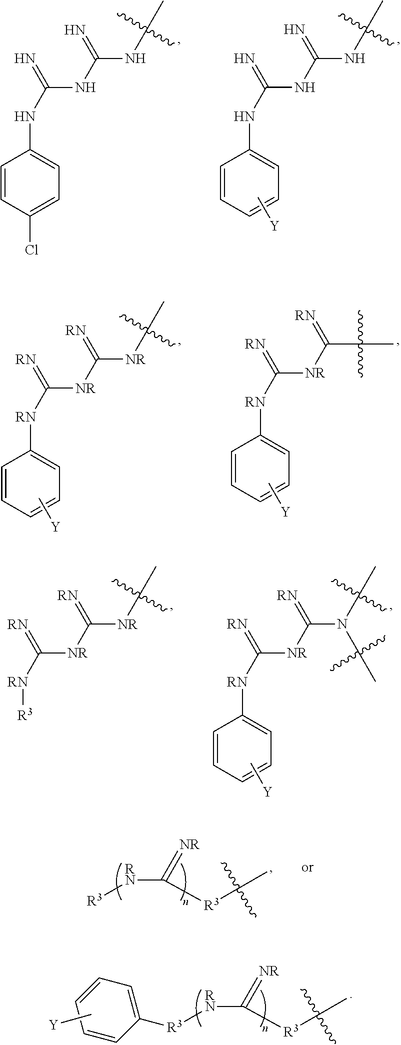

In some embodiments, a guanidine derivative may include a moiety having a structure (including a salt of the structures)

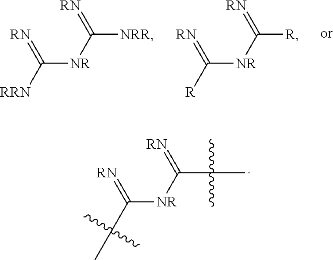

In some embodiments, a guanidine derivative may include an amidine moiety.

In some embodiments, a pharmaceutically active agent may include an anti-viral agent.

In some embodiments, a pharmaceutically active agent may include a topical agent.

In some embodiments, a pharmaceutically active agent may include an antifungal agent.

In some embodiments, a pharmaceutically active agent may include antimicrobial agents.

In some embodiments, an example of a compound 104b may include compounds having a general structure:

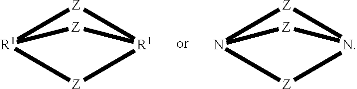

Z may include

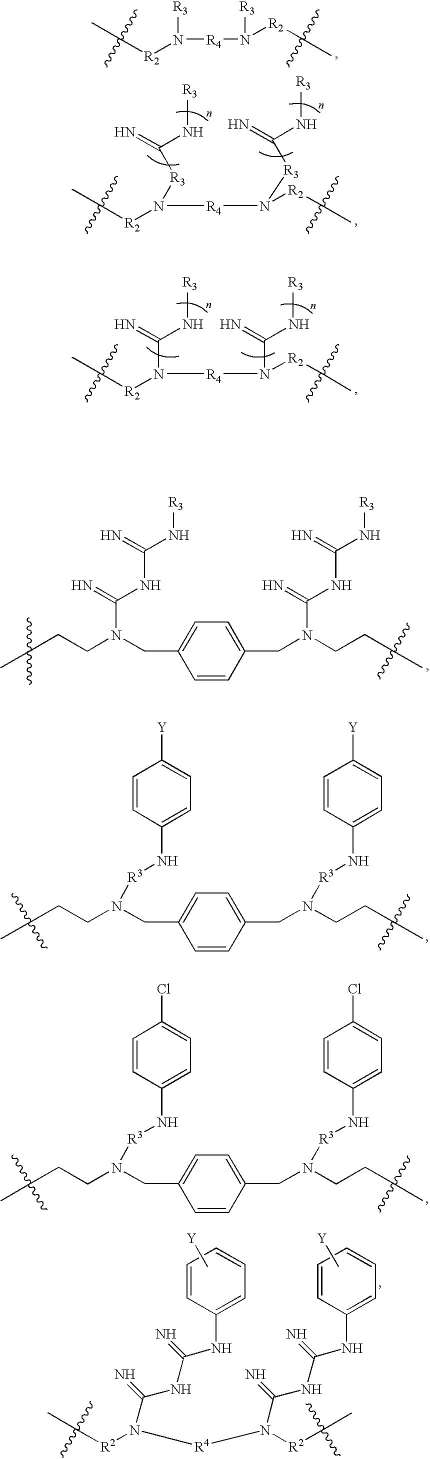

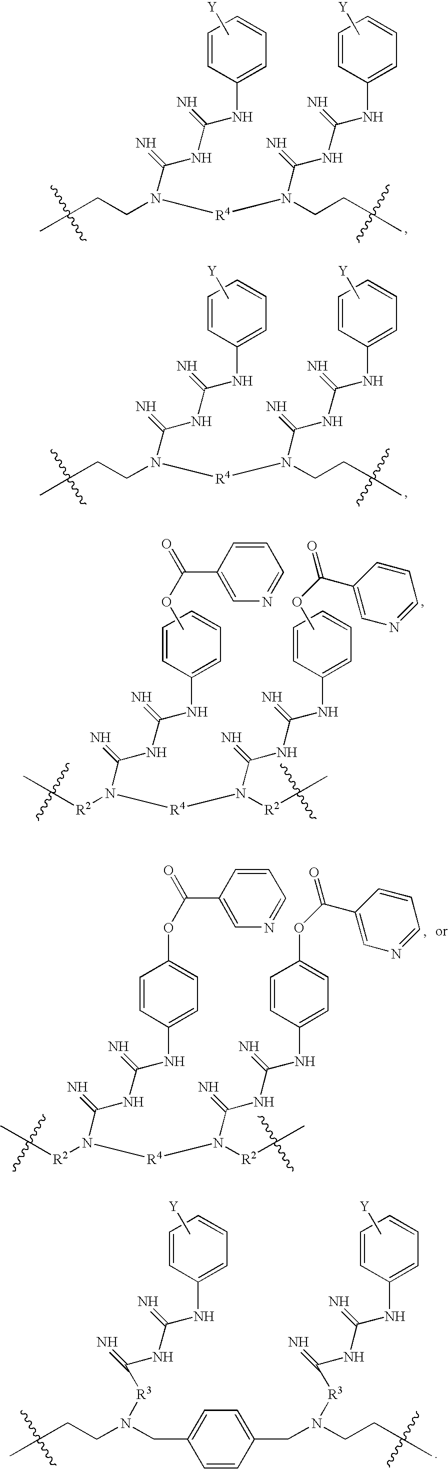

In some embodiments, Z may include at least two bridges.

In some embodiments, a chemical composition may include a chemical compound, wherein the chemical compound has a general structure:

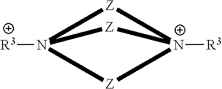

including combinations of Z, X and/or NaOAc as R³ or

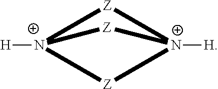

Z may include

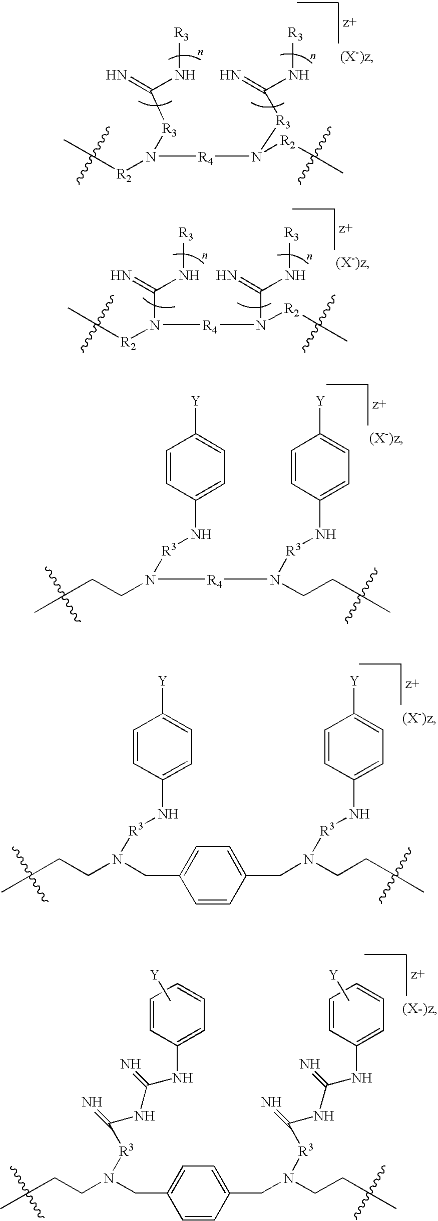

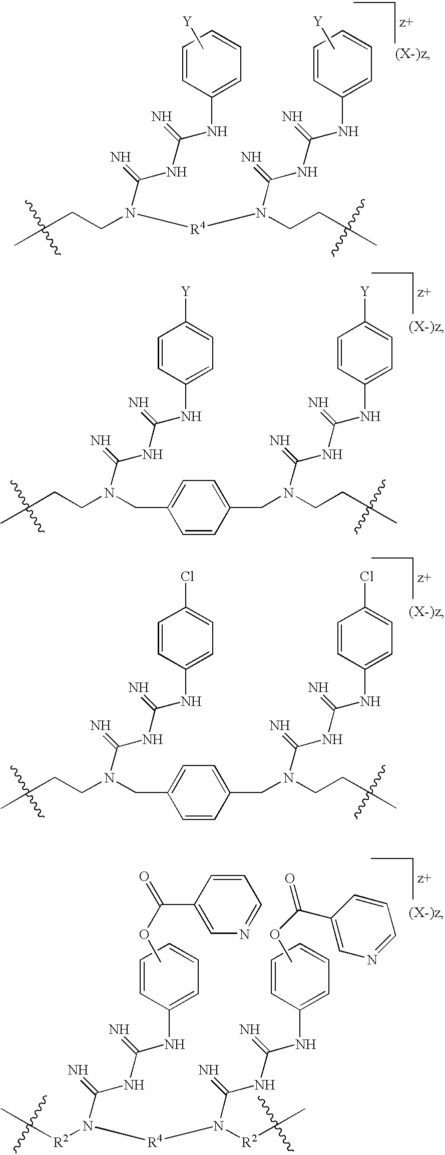

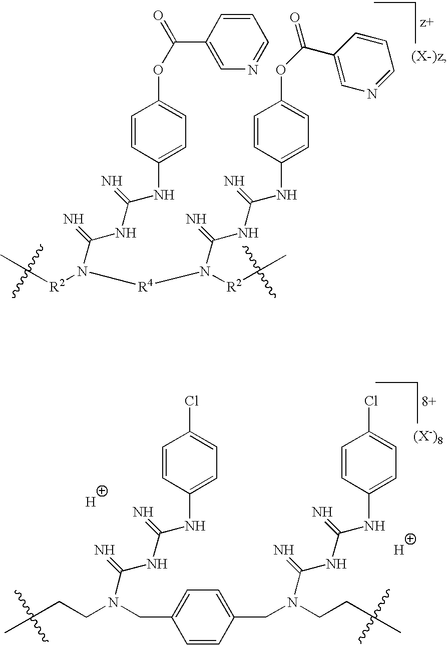

including combinations of Z, X and/or NaOAc,

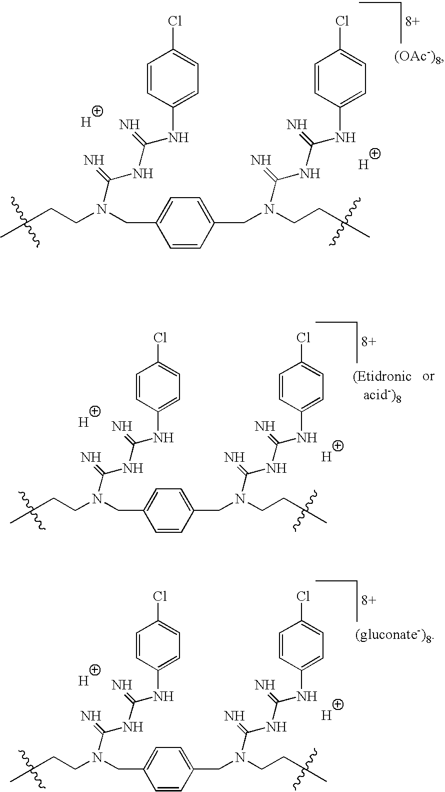

R₃ may include any substituent as described herein in relation to similar bridged polycyclic compounds. R₃ may include aryl, substituted aryl, alkyl, substituted alkyl, and/or hetero atom containing groups. In some embodiments, R₃ may include

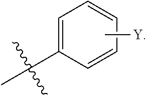

Y may include, for example a halogen (e.g., Cl), an alcohol, or a pharmaceutical active agent (e.g. nicotinic acid, nicotinic acid derivative). Y may include aryl, substituted aryl, alkyl, and/or substituted alkyl. X may include a counter ion. X may include a pharmaceutically active agent (e.g., Etidonate, Butyrate, Pinolenate, Hydroxycitrate). X may include a polymer a multi-functional counterion (e.g. a niconic ester derivative of polyvinyl acetate-co crotonic acid ester and the like). In some embodiments, R₃ may include

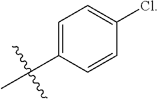

In some embodiments, R₃ may include a guanidine moiety (e.g., guanidine, guanidine derivative) and/or a substituted guanidine moiety. In some embodiments, R₃ may include a halogenated aryl group

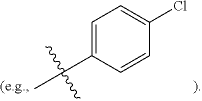

n may range from 1-10, 2-8, 2-4, 3-6, 2-3, or 1-3. In some embodiment, n may be 2. In some embodiments, a z may represent a charge on the chemical compound and an appropriate number of counterions. z may range from 1-16, 2-14, 6-14, 8-14, or 12-20. In some embodiments, y may represent a number of bridges coupling the Nitrogens of the chemical compound. y may range from 3-8, 3-5, or 3-4. In some embodiments, R³ may include one or more pharmaceutically active agents. Pharmaceutically active agents may include functional groups such as amino, quaternary ammonium moieties, and/or guanidine. Such functional groups may be coupled to or part of an alkyl-aryl, substituted alkyl, and/or substituted aryl. For example, an amino group may be coupled to a substituted aryl group or coupled to a substituted alkyl group.

In some embodiments, compounds such as 104b may include salts of the compounds. Salts may include organic and/or inorganic counterions. Counterions may include any of the examples described herein. In some embodiments, a salt of 104b may include an acetate counterion. A salt of 104b (e.g., 10-24) may include a charge from 1-20, 1-14, 4-14, 6-14, 4-10, or 4-8.

In some embodiments, a compound 103 may have a general structure

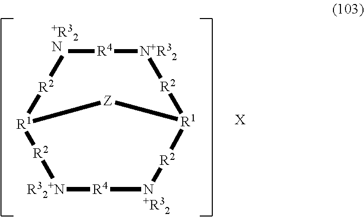

In some embodiments, R¹ may be independently alkyl, substituted alkyl, aryl, substituted aryl, N, N⁺R³, heterocycle, or substituted heterocycle. R² may be independently alkyl, substituted alkyl, aryl, substituted aryl, heterocycle, substituted heterocycle, covalent bond, or alkene. R³ may be independently a pharmaceutically active agent, alkyl, substituted alkyl, aryl, substituted aryl, heterocycle, substituted heterocycle, alkene, ether, PEG, or PEI. In some embodiments, R³ may include one or more pharmaceutically active agents. Pharmaceutically active agents may include functional groups such as amino, quaternary ammonium moieties, and/or guanidine. Such functional groups may be coupled to or part of an alkyl-aryl, substituted alkyl, and/or substituted aryl. For example, an amino group may be coupled to a substituted aryl group or coupled to a substituted alkyl group. R⁴ may be independently alkyl, substituted alkyl, aryl, substituted aryl, N⁺R³, heterocycle, substituted heterocycle, alkyl ether, PEG, PEI, ether, contains boron, or alkene. R⁴ may independently include amide, alcohol, ester, sulfonamide, or sulfanilamide. R⁴ may be independently alkyl, substituted alkyl, aryl, substituted aryl, heterocycle, substituted heterocycle, ether, amide, alcohol, ester, sulfonamide, sulfanilamide, or alkene. X may be one or more counter ions. Z may include at least one bridge.

In some embodiments, at least one of the bridges may be —R²—N⁺R³₂—R⁴—N⁺R³₂—R²—.

Each bridge may independently couple R¹ to R¹. In some embodiments, at least one of the bridges may be —R²—NR³—R⁴—N⁺R³₂—R²—. Each bridge may independently couple R¹ to R¹. In some embodiments, at least one of the bridges may be —R²—NR³—R⁴—NR³—R²—. Each bridge may independently couple R¹ to R¹. In some embodiments, at least one of the bridges may be —R²—N═R⁴═N—R²—. Each bridge may independently couple R¹ to R¹.

For example when Z is 1 compound 103 may be a compound 104 having a general structure

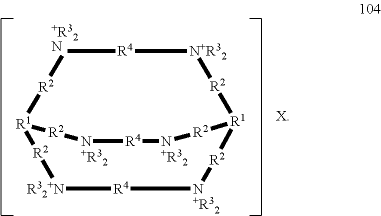

When, for example, Z is 2 a compound 103 may be a compound 104a having a general structure

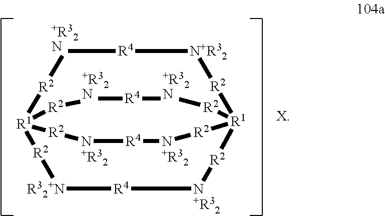

In some embodiments, a compound 104 may have a general structure

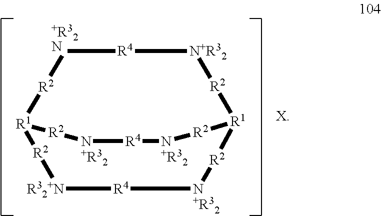

In some embodiments, R¹ may be alkyl, substituted alkyl, aryl, substituted aryl, N⁺R³, heterocycle, or substituted heterocycle. R² may be alkyl, substituted alkyl, aryl, substituted aryl, N⁺R³, heterocycle, substituted heterocycle, covalent bond, or alkene. R³ may be alkyl, substituted alkyl, aryl, substituted aryl, heterocycle, substituted heterocycle, alkyl ether, PEG, PEI, or alkene. In some embodiments, R³ may include one or more pharmaceutically active agents. Pharmaceutically active agents may include functional groups such as amino, quaternary ammonium moieties, and/or guanidine. Such functional groups may be coupled to or part of an alkyl-aryl, substituted alkyl, and/or substituted aryl. For example, an amino group may be coupled to a substituted aryl group or coupled to a substituted alkyl group. R⁴ may be alkyl, substituted alkyl, aryl, substituted aryl, N⁺R³, heterocycle, substituted heterocycle, alkyl ether, PEG, PEI, ether, or alkene. R⁴ may include amide, alcohol, ester, sulfonamide, or sulfanilamide. X may be one or more counter ions.

In some embodiments, counterions may include one or more halogens (e.g., Br, Cl, I, etc.). A specific embodiment of a halogen counterion may include Iodine which has proven as a more effective counterion for antimicrobial compounds. As has been discussed herein, counterions may affect the properties of the chemical compound and subsequent composition. Boron based counterions may increase certain antimicrobial properties (e.g., BF₄⁻).

In some embodiments, salts of specific counterions may be added to a pharmaceutical composition to increase the effectiveness of the composition. For example, any of the counterions described herein for use in making the bridged polycyclic compound (e.g., counterions which increase a pharmaceutically active agent's effectiveness of the compound), may be added to the composition later (e.g., as a salt such as sodium or potassium tetrafluoroborate). In some embodiments, a combination of the two strategies may be used, additionally allowing for two or more different counterions or salts to be included in the final formulation of the composition. Each of the counterions and/or salts may increase the effectiveness of the composition in a different manner. Other examples of counterions (which may be added as an appropriate salt later in an ion exchange or a desired salt may be used during synthesis of the bridged polycyclic compound) may include an anion, a polymer, a monomer, a halogen, an iodine, a bromine, a chlorine, a triflate, a tosylate, a boron, a borate, tetrafluoroborate, a nitrogen containing group, a nitrate, a halogen, a hexafluorophosphate, an acetate, or an NTf₂ (wherein Tf is bis(trifluoromethanesulfonyl)imide).

In some embodiments, a compound may include one or more guest molecules coupled to the compound such as compound 106 having a general structure

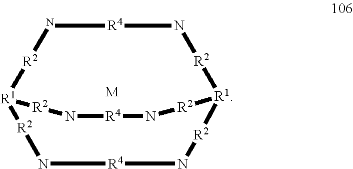

In some embodiments, R¹ may be alkyl, substituted alkyl, aryl, substituted aryl, N, N⁺R³, heterocycle, or substituted heterocycle. R² may be alkyl, substituted alkyl, aryl, substituted aryl, N⁺R³, heterocycle, substituted heterocycle, covalent bond, or alkene. R³ may be alkyl, substituted alkyl, aryl, substituted aryl, heterocycle, substituted heterocycle, alkyl ether, PEG, PEI, or alkene. In some embodiments, R³ may include one or more pharmaceutically active agents. Pharmaceutically active agents may include functional groups such as amino, quaternary ammonium moieties, and/or guanidine. Such functional groups may be coupled to or part of an alkyl-aryl, substituted alkyl, and/or substituted aryl. For example, an amino group may be coupled to a substituted aryl group or coupled to a substituted alkyl group. R⁴ may be alkyl, substituted alkyl, aryl, substituted aryl, N⁺R³, heterocycle, substituted heterocycle, alkyl ether, PEG, PEI, ether, or alkene. M may include one or more guest molecules associated with one or more portions of compound 107 (e.g., amines). M may be one or more metals. M may include silver, zinc, copper, gold, calcium, nickel, cobalt, barium, strontium, lead, lanthanum, iron, manganese, cadmium, magnesium, yttrium, lanthanum, cesium, praseodymium, neodymium, europium, gadolinium, terbium, dysprosium, holmium, erbium, thulium, ytterbium, or alkaline earth metals or cesium. In some embodiments, M may include organic cation salts as templates (e.g., trimethyl ammonium, etc.). M may include light activated elements such that an antiviral or anticancer property of M is increased. X may be one or more counter ions.

In some embodiments, M may be one or more guest molecules. X may be one or more counter ions. M (e.g., Ag+ counter ion) may bind thereby keeping M in close proximity (e.g., F-ions have been reported and verified by x-ray single crystal structure to bind in ammonium salt cavitands). An anion may bind to an ammonium thus affording a close association of the cation counterion. In some embodiments, M may pi-bond coordinate to R₄ (e.g., aryl) or a heterocycle binding (e.g., pyridiyl R₄ nitrogen to a Ag+ or a phenol —OH or O— binding to the Ag+).

In some embodiments, a compound may include one or more guest molecules coupled to the compound such as compound 108 having a general structure

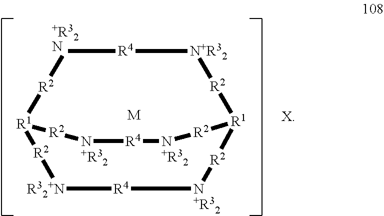

In some embodiments, R¹ may be alkyl, substituted alkyl, aryl, substituted aryl, N⁺R³, heterocycle, or substituted heterocycle. R² may be alkyl, substituted alkyl, aryl, substituted aryl, N⁺R³, heterocycle, substituted heterocycle, covalent bond, or alkene. R³ may be alkyl, substituted alkyl, aryl, substituted aryl, heterocycle, substituted heterocycle, alkyl ether, PEG, PEI, or alkene. R⁴ may be alkyl, substituted alkyl, aryl, substituted aryl, N⁺R³, heterocycle, substituted heterocycle, alkyl ether, PEG, PEI, ether, or alkene. M may be one or more metals. M may include silver, zinc, copper, gold, calcium, nickel, cobalt, barium, strontium, lead, lanthanum, iron, manganese, cadmium, magnesium, yttrium, lanthanum, cesium, praseodymium, neodymium, europium, gadolinium, terbium, dysprosium, holmium, erbium, thulium, ytterbium, or alkaline earth metals or cesium. In some embodiments, M may include organic cation salts as templates (e.g., trimethyl ammonium, etc.). M may include light activated elements such that an antiviral and/or anticancer property of M is increased. X may be one or more counter ions.

It should be understood that any of the compounds depicted herein may or may not have one or more metals coupled to the structure. For example, a structure depicted with a metal associated with the compound also includes a compound without a metal associated with the compound. A structure depicted without a metal associated with the compound also includes a compound with a metal associated with the compound. Although in many instances metals depicted herein are shown positioned within a space defined by compounds described herein, this should not be seen as limiting, metals may be coupled (e.g., complexed to) to a compound along an outer surface of the compound.

Metals may include any elements in the periodic chart designated as metals, known to one skilled in the art. In some embodiments, metals may include any cationic metal known to one skilled in the art (e.g., Zn, Cu, Au, Ag, Cs, Mn, Mg, Ca, Ni, Co, etc.). In some embodiments, metals may include metals which have antiviral and/or anticancer properties and/or anti-inflammatory properties (e.g., Ag, Zn, etc.). In some embodiments, metals may function to couple one or more atoms or molecules within a compound (e.g., compound 108) and/or to the surface of the compound. In some embodiments, one or more metals coupled to a compound may include one or more inorganic/organometallic compounds. A compound (e.g., a bridged polycyclic compound) may include two or more different metals coupled (e.g., associated in some way) to the compound. In some embodiments, a metal may be coupled to a bridged polycyclic molecule.

In some embodiments, R¹ may be N⁺(1-22C alkyl), N⁺(1-12C alkyl), N⁺(1-6C alkyl), N⁺(6C alkyl), N⁺R³,

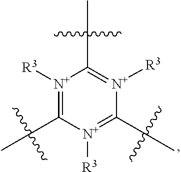

cyclam, aza crown ether, tris ethylamine N substituted cyclam, or

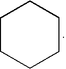

In some embodiments, R² may be 1-2C alkyl, 1-6C alkyl, 2-4C alkyl, CH₂, or a bond (e.g., covalent, ionic) between R¹ and a N of, for example, compound 108.

In some embodiments, R³ may be hydrophobic or hydrophilic. R³ may be 1-3C alkyl, 4-5C alkyl, 6-10C alkyl, 7-9C alkyl, 10-22C alkyl, 15-22C alkyl, 6-10C alkyl ether, 7-9C alkyl ether, methyl, PEI (polyethyleneimine), or PEG (polyethyleneglycol). R³ may be 6C alkyl. R³ may be a polymer. R³ may be an oxazoline polymer.

In some embodiments, R⁴ may include alkyl or substituted alkyl.

In some embodiments, R⁴ may be an aryl, substituted aryl, heterocycle, or substituted heterocycle. R⁴ may be

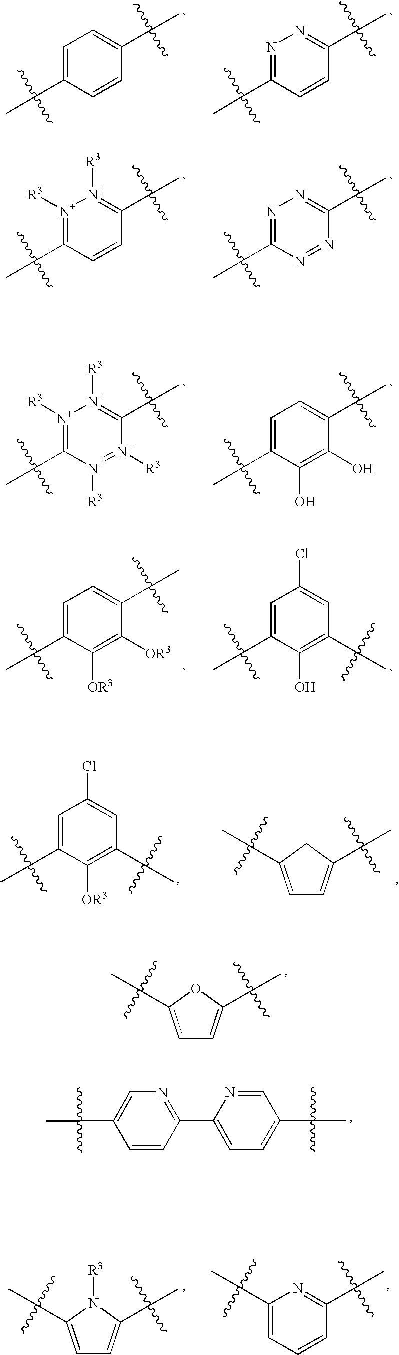

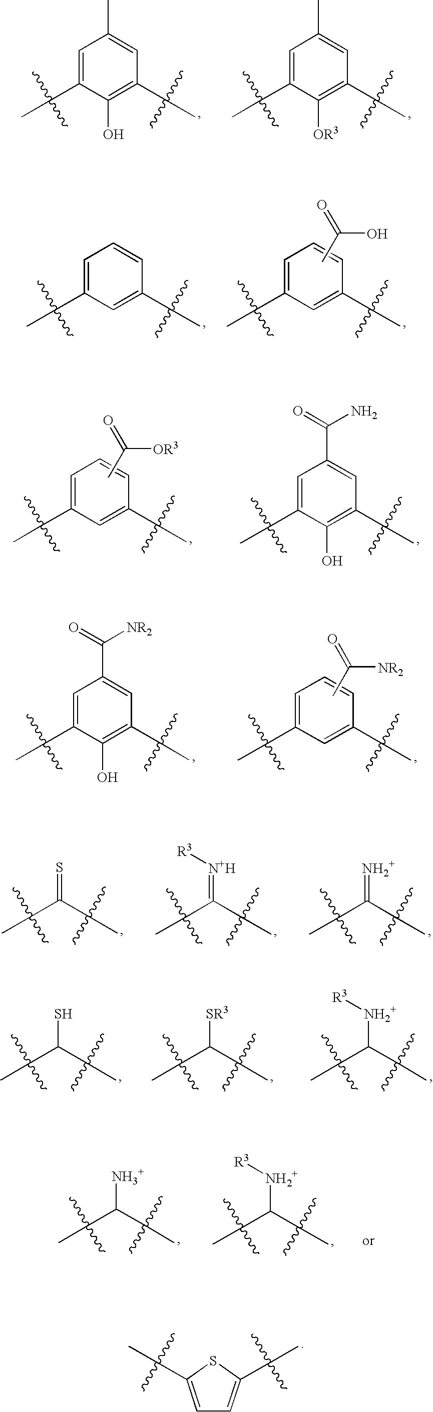

Forming one or more portions of a compound from one or more aromatic rings may provide advantages. Advantages may include providing rigidity to the compound enhancing the stability of the compound. Aromatic rings may facilitate the self-assembly of the constituent parts of the compound. Other advantages may include pie stacking of compounds relative to one another or of “guests” positioned within the compound. A substituted aryl or heterocycle may include moieties (e.g., N) which bind to other elements (e.g., metals such as silver) or molecules. R may include substituents (e.g., R³) which effect properties of a compound as a whole (e.g., hydrophobicity, hydrophilicity, self-cleaning, antimicrobial, cross-coupling properties).

In some embodiments, a compound 108 may include an embodiment such as compound 110 having a general structure

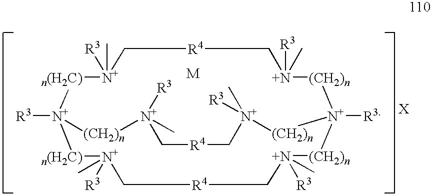

In some embodiments, R³ may be alkyl, substituted alkyl, aryl, substituted aryl, heterocycle, substituted heterocycle, alkyl ether, PEG, PEI, or alkene. R⁴ may be alkyl, substituted alkyl, aryl, substituted aryl, N⁺R³, heterocycle, substituted heterocycle, alkyl ether, PEG, PEI, ether, or alkene. M may include one or more “guest” molecules (e.g., one or more metals). X may be one or more counter ions.

In some embodiments, a compound 104 may include an embodiment such as compound 111 having a general structure

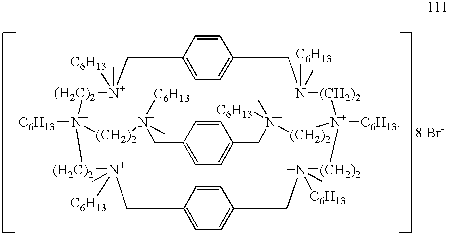

In some embodiments, a compound 104 may include any number of combination of embodiments such as compound 113 having a general structure

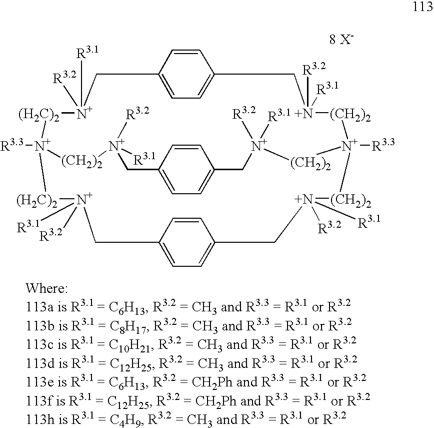

In some embodiments, a compound 104 may include a an embodiment such as compound 114 having a general structure

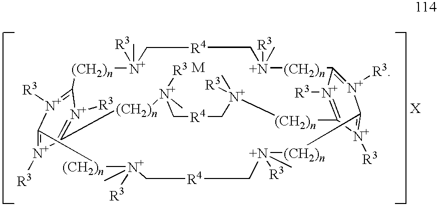

In some embodiments, R³ may be alkyl, substituted alkyl, aryl, substituted aryl, heterocycle, alkyl-aryl, substituted heterocycle, alkyl ether, PEG, PEI, or alkene. In some embodiments, R³ may include one or more pharmaceutically active agents. Pharmaceutically active agents may include functional groups such as amino, quaternary ammonium moieties, and/or guanidine. Such functional groups may be coupled to or part of an alkyl-aryl, substituted alkyl, and/or substituted aryl. For example, an amino group may be coupled to a substituted aryl group or coupled to a substituted alkyl group. R⁴ may be alkyl, substituted alkyl, aryl, substituted aryl, N⁺R³, heterocycle, substituted heterocycle, alkyl ether, PEG, PEI, ether, or alkene. M may be one or more metals. X may be one or more counter ions.

Substituents (e.g., R³) may be configured to perform a variety of functions. By using different substituents, properties of a compound such as the bridged polycyclic compounds described herein may be customized to meet a particular industrial and/or individual's need. For example, R³ may be hydrophobic or hydrophilic depending upon the specific property needed.

In some embodiments, a substituent (e.g., R³) may be multifunctional such that it imparts two or more properties to a formed compound. For example a substituent (e.g., R³) may function to increase the hydrophilicity of a compound, as well as, function as a cross-coupling reagent to cross-link compounds to one another under appropriate conditions (e.g., a substituent may include one or more heteroatoms within its structure such as N, O, and S).

In some embodiments, substituents such as R³ may function to enhance hydrophobicity and/or lipophilicity. Depending upon the needs of a customer the hydrophobicity/lipophilicity of a compound may be increased. Adjusting the hydrophobicity/lipophilicity of a compound may consequently adjust the solubility of the compound in a particular solvent and/or matrix. Increasing the liphophilicity of a substituent (e.g., R³) coupled to an ammonium salt may increase the anti-microbial activity of a compound. In some embodiments, a compound may have a minimum inhibitory concentration (MIC) of less than 900 μM, of less than 600 μM, or of less than 300 μM. A discussion of relationship between substituent chain length and antimicrobial activity of quaternary ammonium salts may be found in Pernak et al., “Synthesis and anti-microbial activities of some pyridinium salts with alkoxymethyl hydrophobic group” Eur. J Med. Chem. 36 (2001) 899-907, which is incorporated by reference as if fully set forth herein.

The relationship between substituent chain length and antimicrobial activity is demonstrated in tests conducted on 113a, 113b, 113d, 113e, and 113h detailed herein in the Examples portion. A series of bridged polycyclic compounds were synthesized wherein different substituents were coupled to the quaternary ammonium moieties. Substituents included C1, C4, C6, C8, C12, and benzyl in combinations of C1 with C4, C6, C8, and C12, as well as, combinations of benzyl with C6 and C12. Time kill and residual surface tests of the antimicrobial strength of the compounds were tested against examples of gram + bacteria (e.g., Staphylococcus aureus, most common surgical wound infection), gram − bacteria (e.g., Escherichia coli, most commonly acquired hospital infection), and fungus (e.g., Aspergillus niger, a toxic black mold found in residences). Of the various alkyl chains combined with C1 tested, the C6,C1 compound tested as the strongest antimicrobial compound. When the test results of the C6,C1 were compared to the benzyl derivatives, once again, the C6,C1 derivative tested as the overall strongest antimicrobial.

The 113a C6C1 compound is unique in regards to the relatively short alkyl chain vs. known quaternary antimicrobials and high antimicrobial activity. Discrete quaternary ammonium or pyridinium antimicrobial molecules usually possess long alkyl chains. The most effective discrete (e.g., noncyclic) quaternary ammonium or pyridinium salt antimicrobials have an alkyl chain length between 12 and 18 carbon atoms as described by Thorsteinsson, T. et. al. “Soft Antimicrobial Agents: Synthesis and Activity of Labile Environmentally Friendly Long Chain Quaternary Ammonium Compounds” J. Med. Chem., 2003, Vol. 46, 4173-4181, which is incorporated by reference as if fully set forth herein.

In general it is known in the art that quaternary ammonium compounds are effective biocidal agents when they possess an alkyl chain with at least eight carbon atoms (Chen, C. Z. et al “Recent Advances in Antimicrobial Dendrimers”, Advanced Materials 2000, Vol. 12, no. 11, 843-846, which is incorporated by reference as if fully set forth herein). In a study of dendrimer quaternary ammonium salts, dendrimer biocides carrying C₁₀ alkyl chains were the most potent (Chen, C. Z. et. al. “Quaternary Ammonium Functionalized Poly(propylene imine) Dendrimers as Effective Antimicrobials: Structure-Activity Studies” Biomacromolecules, 2000, Vol. 1, No. 3, 473-480, which is incorporated by reference as if fully set forth herein).

Typically, non-discrete polymers are some of the only antimicrobials to show any appreciable antimicrobial activity with alkyl groups of <8 carbons. However, non-discrete polymers (e.g. polyethyleneimine quaternary ammonium containing polymers) demonstrated weaker overall antimicrobial activity in antimicrobial residual surface tests (Lin, J. et al. “Making thin polymeric materials, including fabrics, microbicidal and also water-repellent” Biotechnology Letters, 2003, Vol. 25, 1661-1665, which is incorporated by reference as if fully set forth herein).

Furthermore, the straightforward route and synthesis efficiency makes bridged polycyclic compounds (e.g., 113a) more attractive from a manufacturing standpoint over the more laborious methods required for typical dendrimer synthesis. Both bridged polycyclic compounds (e.g., 113a) and dendrimers have the advantage of being polyvalent (multiple positively charged sites on one molecule to attract microbes) affording increased activity vs. traditional discrete quaternary ammonium salts (S. L. Cooper et. al. U.S. Pat. No. 6,440,405). However, the dendrimer synthesis requires large volumes of solvents/reagents relative to obtained product and long periods of time (days) to synthesize as described by S. L. Cooper et. al. in U.S. Pat. No. 6,440,405 to Cooper et al., which is incorporated by reference as if fully set forth herein. Dendrimers have been discussed as possible cholesterol and ion (e.g., renal therapy) sequestrants in Dhal, P. K. “Biologically active polymeric sequestrants: Design, synthesis, and therapeutic applications” Pure Appl. Chem., vol. 79, No. 9, 1521-1530, (2007), which is incorporated by reference as if fully set forth herein.

In some embodiments, substituents such as R³ may function to enhance hydrophilicity and/or lipophobicity. Depending upon the needs of a customer the hydrophilicity/lipophobicity of a bridged polycyclic compound may be increased. Adjusting the hydrophilicity/lipophobicity of a compound may consequently adjust the solubility of the compound in a particular solvent and/or matrix.

In some embodiments, counter ions for a bridged polycyclic compound may be selected to adjust particular properties of a compound or to introduce new properties to the compound. Adjusting properties of a compound based on a selection of a particular counter ion allows further customization of a compound. In some embodiments, counter ions may include counter ions which have or enhance antimicrobial properties and/or anti-inflammatory properties (e.g., boron, zinc). In some embodiment, counter ions may adjust the hydrophilicity or hydrophobicity of the complex. Counter ions may include metals. Research has held that specific counter ions do affect the antimicrobial activity of quaternary ammonium compounds.

Counter ions may include, but are not limited to, organic, inorganic, or organometallic moieties. Examples of counter ions may include inorganic ions (e.g., halogen ions, such as fluorine, bromine, chlorine and iodine), organic ions (e.g., tosylate, prosylate sulfuric acid, nitric acid and phosphoric acid, and ions of organic acids such as succinic acid, fumaric acid, lactic acid, glycolic acid, citric acid, tartaric acid and benzoic acid), or coordinate type anions (e.g., fluoro sulfate and tetrafluoro borate).

In some embodiments, counter ions may include a hydrophobic organic group (e.g., lauryl sulfate, dodecylbenzene sulphonate, diethylhexyl sulphosuccinate, carboxylic acid derivatives with alkane, alkene or alkyne aliphatic tails such as myristic acid salts, octadecanate, dodecanoic acid salts, oleic acid salts, Palmitoleic acid salts, lauric acid salts, Stearic acid salts, phosphinic acid salts, phosphonic acid salts (i.e. tetradecylphosphonate, hexadecylphosphonate) and dodecylsulphonate, dodecylsulfate anions).

In some embodiments, bridged polycyclic compounds and cationic salts thereof may absorb lipids and remove them from the body, for example, effectively lowering cholesterol or decreasing fat uptake by the body (e.g., a gluconate salt of a polymerized bridged polycyclic compound using D-Gluconic acid or a derivative to form the salt). In some embodiments, bridged polycyclic compounds may include compounds associated with the polycyclic compound which in addition to the bridged polycyclic compounds absorb lipids and remove them from the body, for example, effectively lowering cholesterol or decreasing fat uptake by the body.

A discussion of the increased binding ability of bis-guanide over mono- guanide for lipid binding can be found in David, S. A. “Towards a rational development of anti-endotoxin agents: novel approaches to sequestration of bacterial endotoxins with small molecules” J. Molecular Recognition (2001) vol. 14, 370-387, which is incorporated by reference as if fully set forth herein.

Additionally, bridged polycyclic compounds may be associated with MTP inhibitors, Statin derivatives (e.g., Lipitor, Orlistat), or ezetimibe. Statin acids may be coupled to or form a salt with bridged polycyclic compounds. Statin acids may include pravastatin acid, fluvastatin acid, atorvastatin acid, lovastatin acid, or simvastatin acid. In some embodiments, MTP inhibitors, Triacylglycerol Synthesis inhibitors (e.g., niacin), Statins, etc. may be incorporated as anions as pictured above. This may also provide an additional benefit of a slow release mechanism for the MTP inhibitor or Statins (associated with the bridged polycyclic compounds as an anionic salt upon protonation using the carboxylic acid moiety of the MTP inhibitor and/or statin on the amine of the bridged polycyclic compound; e.g., niacin, butyric acid and/or atorvastatin (Lipitor), etc.).

This can be advantageous as the immediate-release form of nicotinic acid (niacin) is safe for long-term use, however, for example the triacylglycerol synthesis inhibitor nicotinic acid (niacin) has frequent side effects, including: sudden blushing or redness of the face (flushing), which is more common with the immediate-release forms of nicotinic acid; itching; liver problems (hepatotoxicity), especially with the sustained-release form; high blood sugar (hyperglycemia); and too much uric acid in the blood (hyperuricemia), excess uric acid in the blood can lead to gout, gastrointestinal problems (e.g., upset stomach, gas, nausea, vomiting, and diarrhea), dizziness, lightheadedness, or a fast or slow heartbeat. In some embodiments, nicotinic acid may be used as a counter ion to slow down its release. In some embodiments, nicotinic acid may be coupled directly or through a linking agent to a bridged polycyclic compound. Linking agents (e.g., esters) may decompose in vivo such that nicotinic acid and/or derivatives of nicotinic acid are released.

In some embodiments, pharmaceutical agents may be directly coupled to the bridged polycyclic compound. In some embodiments, pharmaceutical agents may be incorporated freely with bridged polycyclic compounds in a pharmaceutical composition. Further descriptions of compounds, such as cationic polymers, for lowering cholesterol are included in U.S. Pat. No. 6,562,329 to Hadvary et al., which is incorporated by reference as if fully set forth herein. Further descriptions of MTP inhibitors for cholesterol lowering, which may be used in combination with compounds described herein, for lowering cholesterol are included in U.S. Pat. No. 6,562,329 to Hadvary et al. Descriptions of MTP and its mechanisms in lipoprotein assembly may be found in Marcil, V. et. al. “Butyrate Impairs Lipid Transport by Inhibiting Microsomal Triglyceride Transfer Protein in Caco-2 Cells” J. Nutr. 2003, 133: 2180-2183, which is incorporated by reference as if fully set forth herein.

In some embodiments, benefits similar to those demonstrated by Tyloxapol found in Chandler, C. E. et al. “CP-346086: an MTP inhibitor that lowers plasma cholesterol and triglycerides in experimental animals and in humans” Journal of Lipid Research, Vol. 44, 1887-1901, October 2003, which is incorporated by reference as if fully set forth herein, (e.g., cholesterol lowering, aid liquification and removal of mucopurulent (containing mucus and pus) bronchopulmonary secretions) may be seen. For example, this may be accomplished by reacting the bridged polycyclic compound secondary amine with an anhydride to give structures similar to those shown below derived from compounds like 302. Further reaction of the carboxylic acid moiety with polyethylene glycol derivatives terminated with alcohols and/or amines (e.g., HO-PEG-OH and/or H₂N-PEG-NH₂, including any alkyl, aryl, or combination of PEG derivatives (or any hydrophilic linker) including any additional heteroatom or carbohydrate functionality) may be used to crosslink and produce polymerized bridged polycyclic compounds via the secondary amine moieties that are functionalized by reaction with an anhydride followed by PEG crosslink via ester and/or amide bonds.

In some embodiments, polymerized and non polymerized bridged polycyclic compounds may include butyrate as a counter ion to the bridged polycyclic compounds. Butyrate may function to inhibit MTP lipid aggregation mechanism and lower cholesterol. The bridged polycyclic compound may function to absorb and remove lipids (LDL) from the body. Descriptions of cholesterol reduction using, for example, butyrate may be found in Hussain, M. M. et. al. “Microsomal triglyceride transfer protein and its role in apoB-lipoprotein assembly” Journal of Lipid Research, 2003, vol 44, 22-32, which is incorporated by reference as if fully set forth herein. In some embodiments, other short chain acids (e.g., propionic acid, C5, C6, etc. carboxylic acids) may be employed for a similar effect.

In some embodiments, bridged polycyclic compounds may include hydroxycitrate (from hydroxycitric acid) as a counter ion to the bridged polycyclic compounds. Hydroxycitric acid which has been shown to inhibit production of fats in the human body. Hydroxycitric acid inhibits production of fat by inhibiting enzymes which turn carbohydrates into fat. Hydroxycitric acid promotes formation of glycogen. Formation of glycogen signals the body to decrease hunger signals effectively acting as a hunger suppressant.

In some embodiments, bridged polycyclic compounds may include pinolenate (from pinolenic acid) as a counter ion to the bridged polycyclic compounds. Pinolenic acid is a fatty acid which occurs naturally in several types of nuts (e.g., siberian pine nuts, korean pine nuts). Studies have demonstrated pinolenic acid may function as an appetite suppressant. Pinolenic acid may function by promoting production of hunger suppressants in the human body (e.g., cholecystokinin, glucagon-like peptide). It has be put forth that Pinolenic acid may function to lower LDL by promoting hepatic LDL uptake.

In some embodiments, two or more different pharmaceutically active agents may be coupled to and/or associated with (e.g., as a counter ion) a bridged polycyclic compound. In some embodiments, using two or more pharmaceutically active agents may allow for the combination of different but related effects (e.g., combining an LDL absorbent and an LDL production inhibitor).

In some embodiments, bridged polycyclic compounds may include acetate (from acetic acid) as a counter ion to the bridged polycyclic compounds.

In some embodiments, a bridged polycyclic salt compound may be employed as a LDL absorbent (by sequestering bile acid). This effect may be combined with an LDL production inhibitor (e.g., nicotinic acid). Nicotinic acid may be coupled to the bridged polycyclic salt compound (e.g., as depicted below, nicotinate may be released via acid hydrolysis in the stomach to promote slow release of the nicotinate). In some embodiments, a third pharmaceutically active agent may be combined with a bridged polycyclic compound, for example, an MTP inhibitor may be used. An MTP inhibitor, such as butyrate may be used as a counter ion (e.g., see schemes below). Compound 9 may be synthesized in a similar fashion as compound 5. Nicotinic acid may be coupled to compound 9 using heat and sulfuric acid. The nicontinate derivative of 9 may then be converted to the freebase and finally converted to the butyrate salt 11.

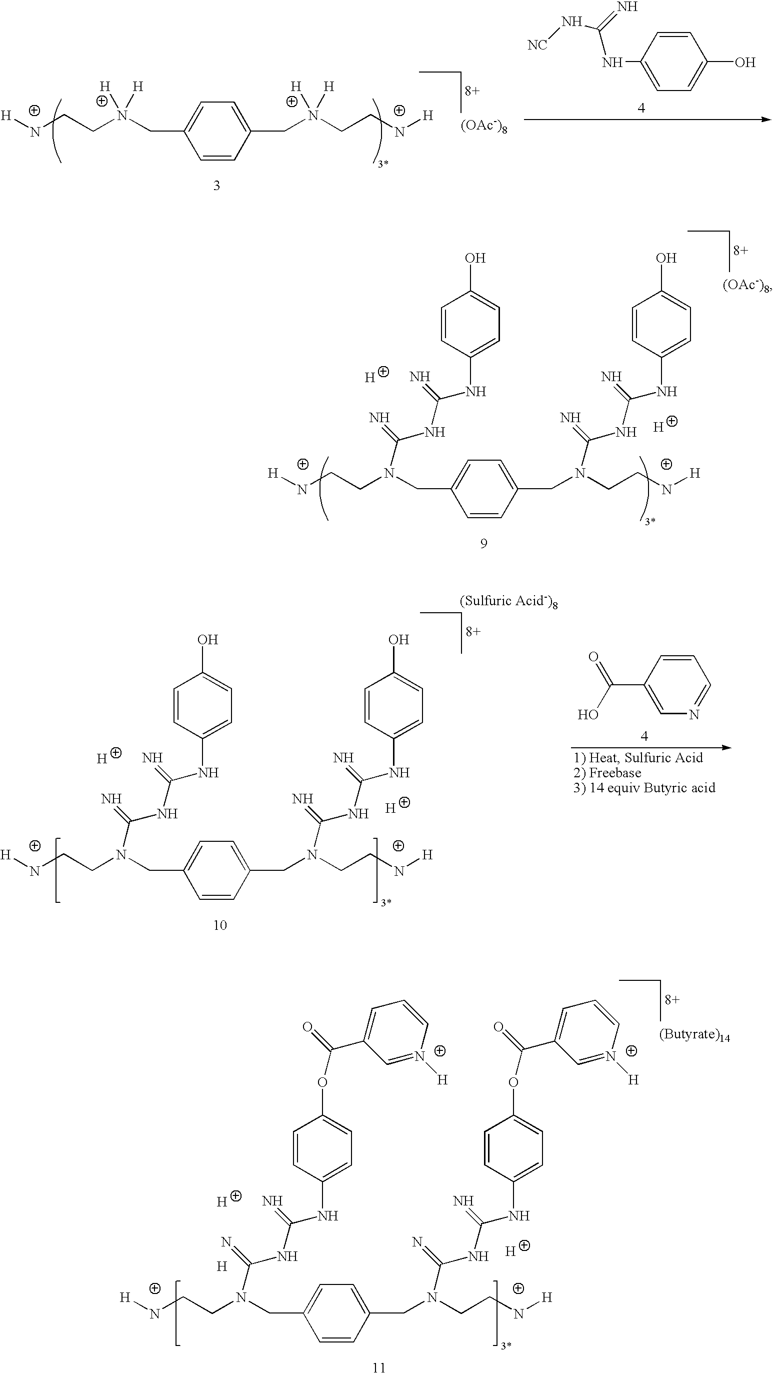

In an alternative synthesis nicotinic acid derivative 11 may be synthesized by reacting the freebase of 9 with the acid chloride derivative (4) of nicotinic acid followed by treatment of butyric acid to form the nicotinic acid derivative 11.

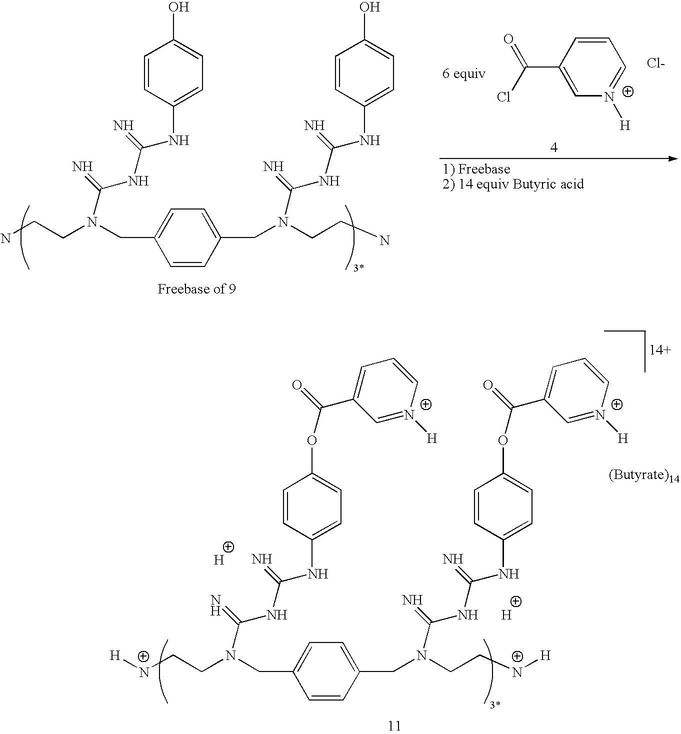

In some embodiments, bridged polycyclic compounds may be polymerized. Polymers incorporating bridged polycyclic compounds may have molecular weights high enough to inhibit systemic absorption when, for example, ingested. The minimum molecular weight, and hence the degree of polymerization of bridged polycyclic compounds, required to inhibit systemic absorption may be relatively low. Nonsystemic polymers may include a minimum degree of polymerization of 3 or greater, 6 or greater, 10 or greater, 20 or greater, or 50 or greater. In some embodiments, an enteric coating may be applied to a composition in order to inhibit absorption and/or premature absorption.

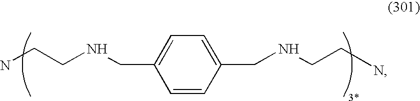

Herein, compound 301 as depicted above is merely a structural short-hand version of the more conventional structural representation of compound 301 depicted as

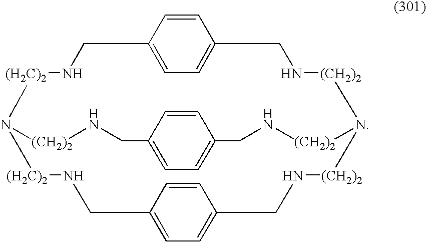

Herein a structure with a * notation next to a subscripted number after a bracket or a parenthesis may denote bridged polycyclic compounds with the subscripted number representing the number of bridges (represented by the compound within the bracket or parentheses). This will function to differentiate the more common usage of this type of notation for polymers and/or oligomers.

In some embodiments, bridged polycyclic compounds may be polymerized in any number of ways known to one skilled in the art. Bridged polycyclic compounds may be polymerized using methods known to polymerize amines. In some embodiments, bridged polycyclic compounds (e.g., compounds 113 herein) may be polymerized via the tertiary amines or the secondary amines.

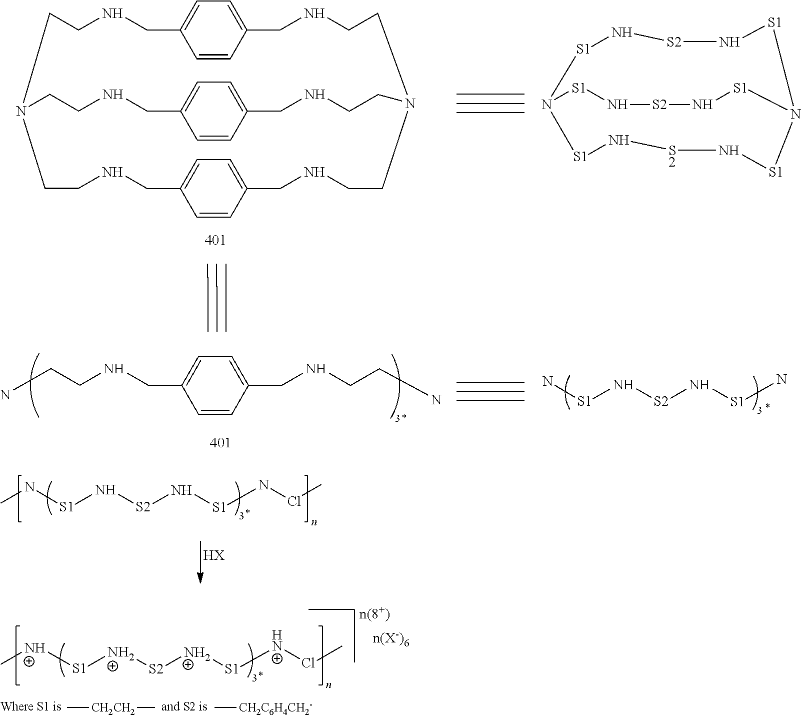

In some embodiments, C1 may be polyethylene glycol (PEG), alkyl, and/or aryl. In some embodiments, X may include niacin, butyrate, a statin (e.g., Atorvastatin, Exetimibe), or other anionic counterion. Any bridged polycyclic compound described herein may be polymerized.

In some embodiments, a polymerized bridged polycyclic compound may be substituted with linkers as described herein such that, for example, more pharmaceutical agents may be coupled to the polymer. For example, guanidine moieties may be used to replace the (H) of (—NH—), then add HX to form the salt of guanidine moiety and the cage amines to give higher overall charge.

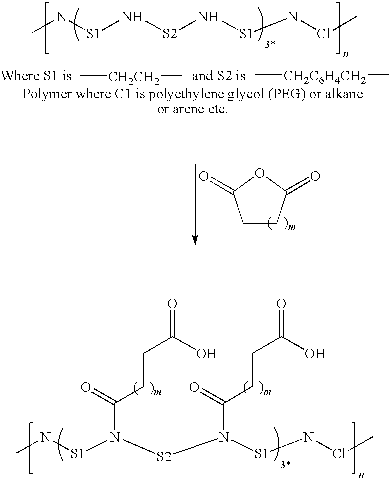

In some embodiments, any acid functionality (represented by Y) may be employed in polymerized bridged polycyclic compounds.

In some embodiments, any guanidine functionality (represented by Y) may be employed in polymerized bridged polycyclic compounds.

In some embodiments, any amide functionality (represented by Y) may be employed in polymerized bridged polycyclic compounds.

In some embodiments, any amide and/or ester functionality (represented by Y) may be employed in polymerized bridged polycyclic compounds.

In some embodiments, any polyethylene glycol functionality (represented by Y) may be employed in polymerized bridged polycyclic compounds.

In some embodiments, any proton with an ionic counterion functionality (represented by Y) may be employed in polymerized bridged polycyclic compounds.

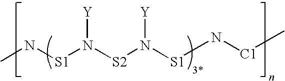

### Synthesis of Bridged Polycyclic Compounds

For commercialization purposes compounds such as bridged polycyclic compounds (and their metal and/or metal oxide coated counterparts) require an efficient and cost effective method of synthesis. In some embodiments, bridged polycyclic compounds may be formed through the self-assembly of two or more compounds to form much larger complex system in fewer steps and more efficiently than traditional stepwise synthetic means.

At the most general level, the words “self-assembly” are used to identify the phenomenon whereby some kind of higher-level pattern emerges from the interactions of multiple simple components. An example of self-assembly from the Stang group is shown in Scheme 1 (Stang, P. J.; Cao, D. H. J. Am. Chem. Soc. 1994, 116, 4981). To set this particular type of self-assembly in its proper context, it should be noted that in the field of chemistry, the term “self-assembly” is used to describe two distinct types of processes. On the one hand, there are assemblies that lead to the formation of essentially infinite arrays, while on the other hand, there are assemblies such as that shown in Scheme 1 that lead to distinct, bounded species. Furthermore, within each of these categories, it is possible to make a further distinction that reflects the scale of organization. For example, for infinite arrays, one may consider processes such as crystallization, where the molecules are ordered at the molecular level (ca. 10⁻⁹ m), or the formation of self-assembled monolayers and bilayers, where there is little order between individual molecules, but a larger scale of organization is evident across say the 10⁻⁶ m level. Likewise, the scale of organization for assemblies leading to distinct species may be broken down into similar categories. It may be noted the self-assembly of macroscale objects (10⁻³ m) is currently being investigated. However, as far as the interaction of molecules to form distinct species goes, it may be considered the formation of micelles and vesicles that constitutes assembly at the 10⁻⁶ m level.

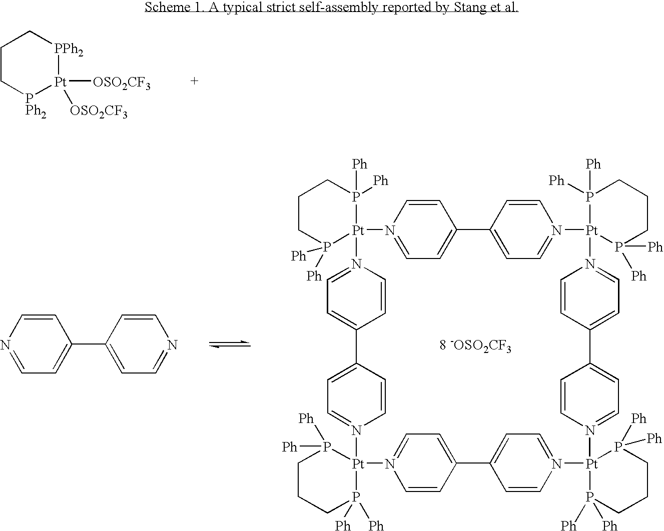

The essential features of chemical assembly processes is that they share a common self-correcting mechanism. In other words, strict self-assemblies are fully reversible, dynamic, systems that lead to a product that represents the global thermodynamic minimum for the system. Sometimes an additive or template is needed to boost the efficiency of the assembly, but this is the only true variable if one is speaking of strict self-assembly. At their cores, strict molecular assemblies consist of subunits, product, and an equilibrium that relates the two.

In some embodiments, self-assembly techniques (e.g., dynamic covalent chemistry) may be employed to synthesize stable compounds, which are themselves large enough to be described as nanoparticles and/or which may be used to form nanoparticles.

Bridged polycyclic compounds represented by compounds 104 and 108 may be synthesized by any means known to one skilled in the art. As has been mentioned, self-assembly may be a useful technique for efficiently synthesizing nanoparticles described herein. In some embodiments, nanoparticles such as compounds 104 and 108 may be formed via self-assembly using Schiff base condensation reactions between amines and aldehydes to form an imine as depicted in Scheme 3. For example, a trifunctional amine (e.g., tris(2-aminoethyl)amine (TREN)) may be reacted with a bifunctional aldehyde (e.g., ethane-1,2-dione (glyoxal)).

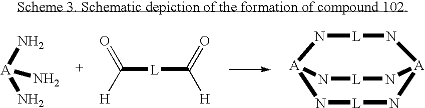

In Scheme 3, the amine depicted is trifunctional and the aldehyde is bifunctional. However, the example depicted in Scheme 3 should not be seen as a limiting embodiment. For example, a Schiff base condensation reaction is depicted in Scheme 4 in which the amine is bifunctional and the aldehyde is trifunctional.

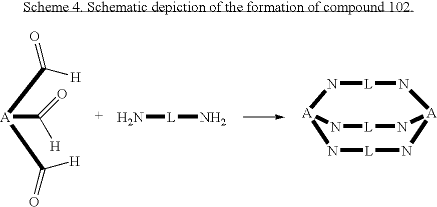

In some embodiments, two different trifunctional molecules may be reacted with one another in order to form an asymmetric adduct. Scheme 4a depicts an embodiment of the formation of an asymmetric adduct.

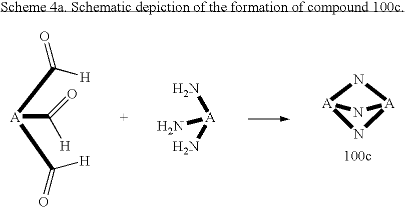

For example, a trifunctional amine (e.g., tris(2-aminoethyl)amine (TREN)) may be reacted with a trifunctional aldehyde (e.g., 1,3,5-aldehyde substituted phenyl). Triethanolamine may be functionalized at the OH with an aminoacid to give N—(CH₂CH₂OC(O)Phenyl(CHO). N—(CH₂CH₂OC(O)Phenyl(CHO) may be reacted with any triamine to give an asymmetric example of a bridged polycyclic compound. A discussion of synthesis techniques for different multifunctional ligands (e.g., trifunctional aldehydes) may be found in Chand et al. “Synthesis of a Heteroditopic Cryptand Capable of Imposing a Distorted Coordination Geometry onto Cu(II): Crystal Structures of the Cryptand (L), [Cu(L)(CN)](picrate), and [Cu(L)(NCS)](picrate) and Spectroscopic Studies of the Cu(II) Complexes” Inorg Chem 1996, Vol. 35, 3380-3387, which is incorporated by reference as if fully set forth herein.

In some embodiments, formation of a bridged polycyclic compound (e.g., Schemes 4, 4a, or 5) may be carried out in an alcohol (e.g., ethanol).

A more specific example of the self-assembly Schiff base condensation strategy depicted in Scheme 3 is depicted in Scheme 5 showing the formation of imine compound 116. Imine compound 116 may be used as an intermediate toward the formation of compound 110.

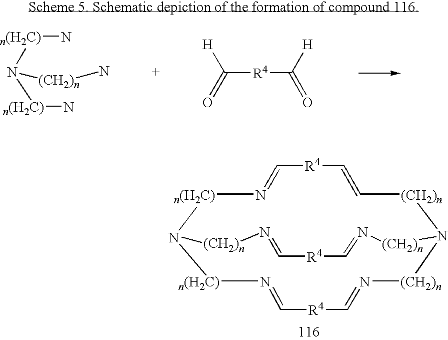

A Schiff base condensation may be carried out using an acid catalyst (e.g., acetic acid). A Schiff base condensation may be carried out using any means known to one skilled in the art. Techniques for amine aldehyde condensations may be found in U.S. Patent Application Publication No. 2004/0267009 to Redko et al., which is incorporated by reference as if fully set forth herein.

In some embodiments, a reduction may be carried out in an alcohol (e.g., ethanol and/or methanol) with with a reducing agent (e.g., sodium borohydride).

In some embodiments, coupling of corner units or corner units and linker units to form bridged polycyclic imine compounds may be carried out in an alcohol (e.g., ethanol) based solvent. In some embodiments, reduction of at least some of the imines may be carried out without any substantial work up directly following the coupling step (e.g., by adding a reducing agent such as sodium borohydride) to form a bridged polycyclic compound.

In the past reactions such as the coupling and reduction steps have been carried out as two totally separate steps involving for example working up (e.g., purifying and isolating) the reaction after the coupling step before the reducing step. One or more of these steps (e.g., the coupling step) have in the past been carried out in for example acetonitrile resulting in a seemingly polymeric substance, followed by an isoxolate extraction. In reality the isoxolate extraction may have been merely driving the reaction towards the bridged polyclic product, by conversion of polymer and oligomer products.

Running the reactions in a solution of heated ethanol results in almost quantitative yields of the desired product without any substantial work up or isolation protocols.

In some embodiments, coupling of corner units or corner units and linker units to form bridged polycyclic imine compounds may be carried out in a green solvent. In some embodiments, a green solvent may include any solvent which is naturally occurring and which has been found not to harm the environment when used on an industrial scale. In some embodiments, a green solvent may include water or an alcohol based solvent (e.g., ethanol). A catalyst may be used to run the reaction in water. In some embodiments a catalyst may include aniline. A similar method is described by Dirksen, A. et al. “Nucleophilic Catalysis of Oxime Ligation” Angew. Chem. Int. Ed. (2006) 45, 7581-7584, which is incorporated by reference as if fully set forth herein.

In another example of functionalizing an amine at least in part defining a bridged polycyclic compound, a functionalized substituent may be coupled to the amine. A functionalized substituent may include an alkyl amine group. A non-limiting example of an alkyl amine may include —CH₂CH₂CH₂NH(CH₂)₅CH₃. The amine may be further functionalized. For example the amine of the alkyl amine may be alklyated such that another quaternary amine is available increasing the antimicrobial activity of the bridged polycyclic compound. The synthesis of such an embodiment is detailed in the Examples section.

In some embodiments, following a reduction to form a bridged polycyclic amine, such as compound 120 or a compound such as compound 301 having a structure

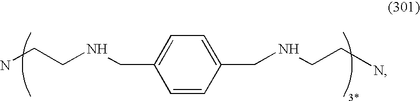

a linking agent (e.g., to couple a pharmaceutically active agent to) or a pharmaceutically active agent may be coupled to a bridged polycyclic amine such as compound (301). Linking agents may be, for example, any of the compounds or reagents identified herein as R³.

Following are some representative example of activating agents and how to synthetically couple them to compounds such as compound 301.

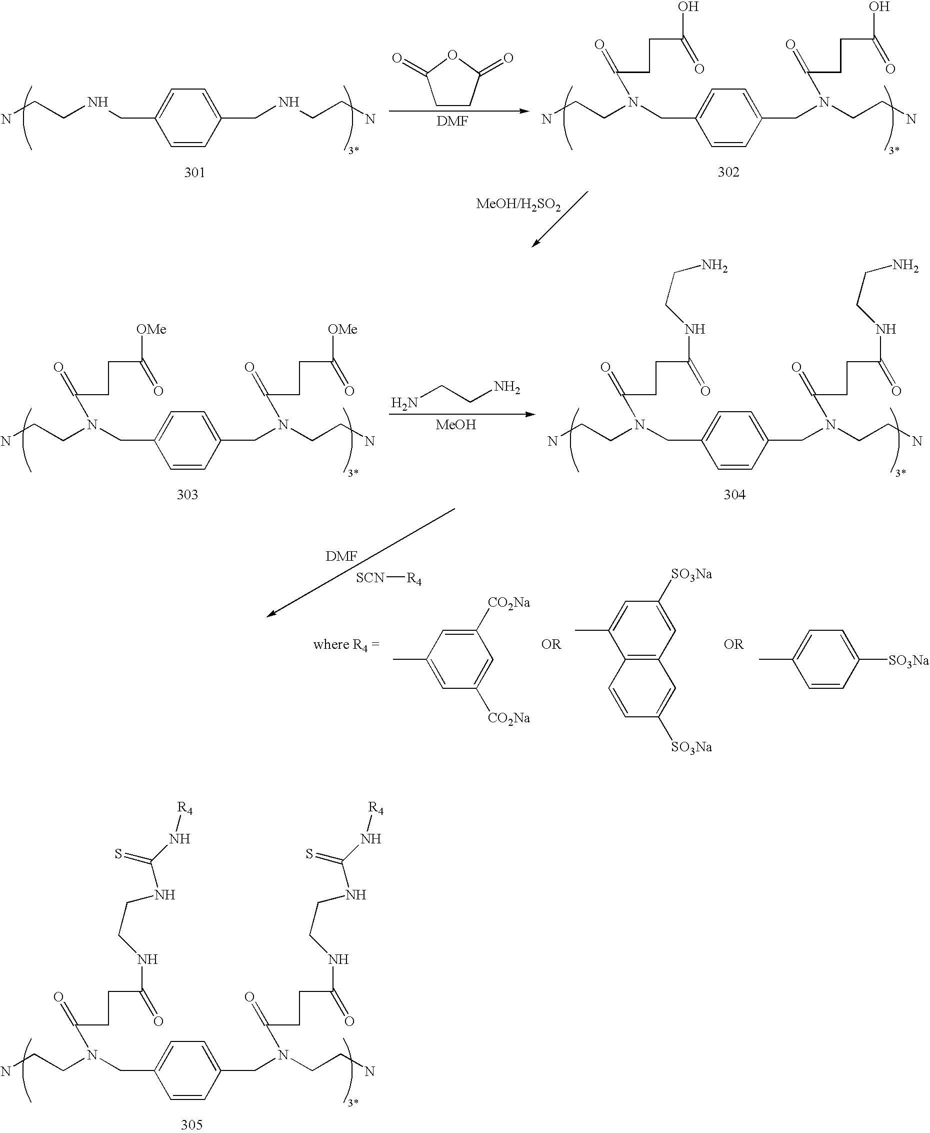

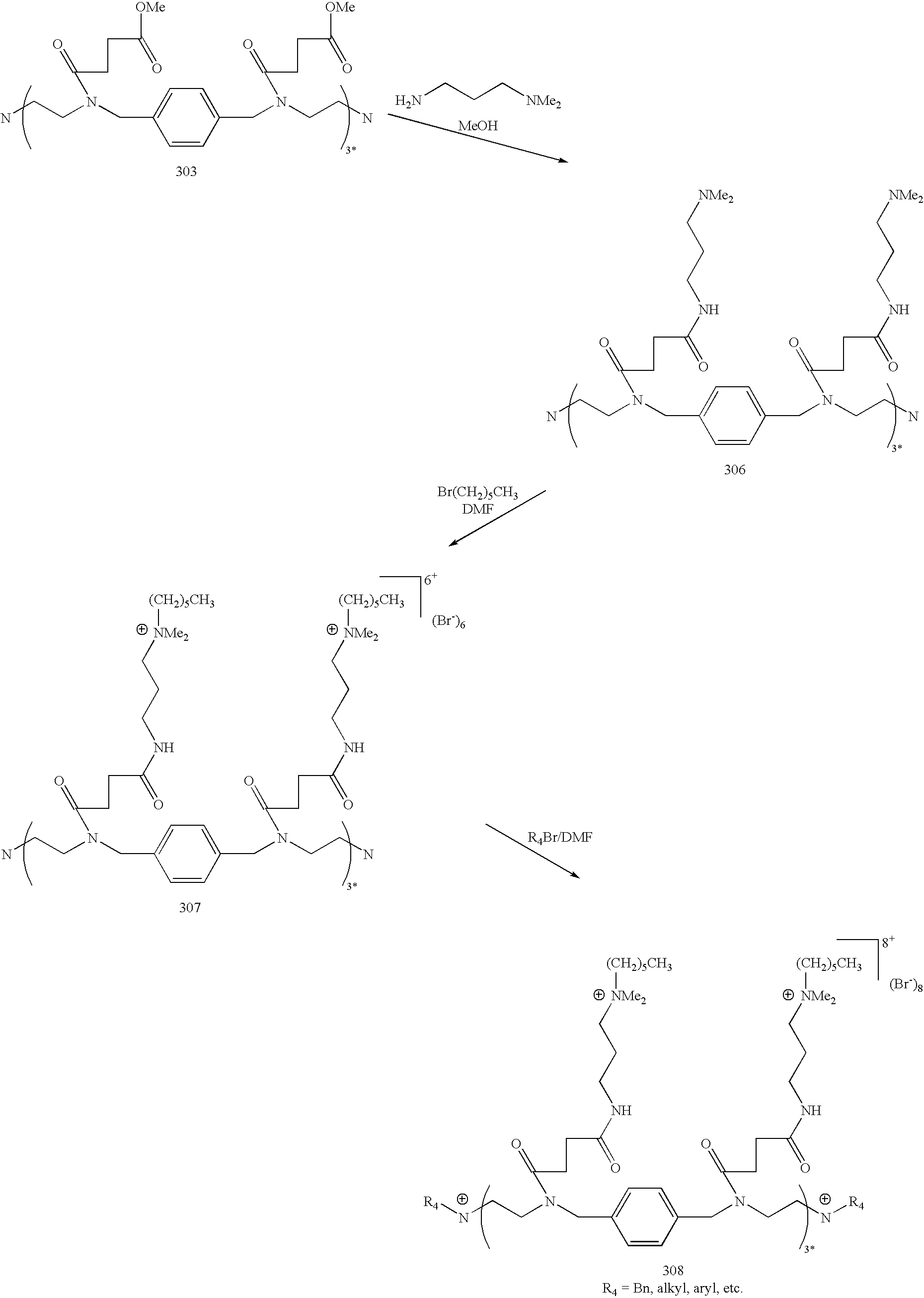

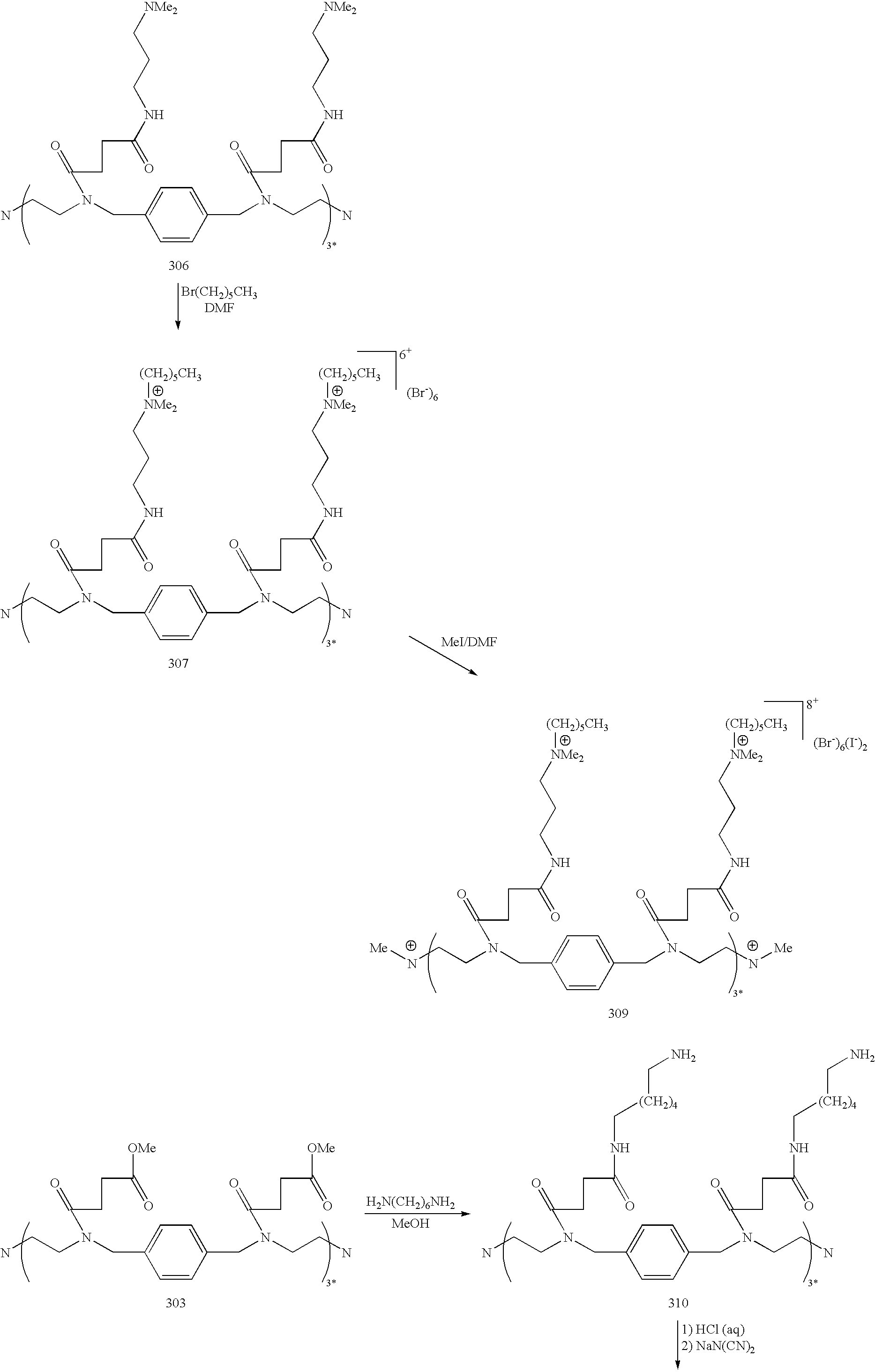

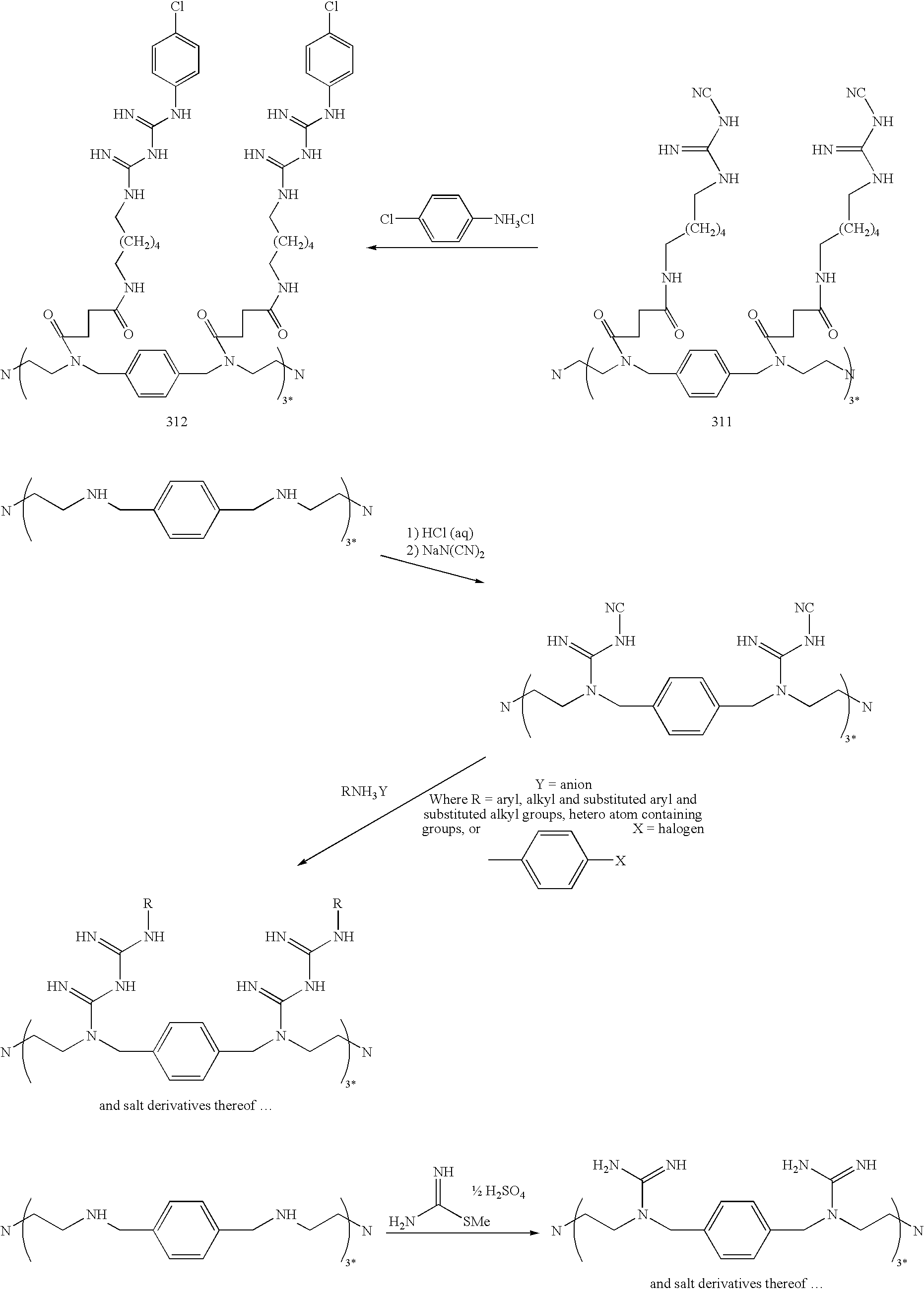

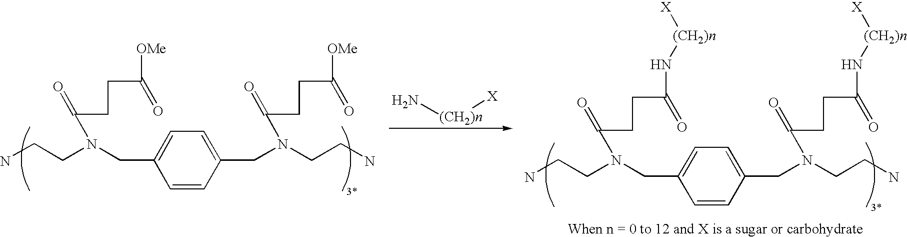

Following is a representative example of how to synthesize a bridged polycyclic compound including a pharmaceutically active agent coupled to the bridged polycyclic compound.

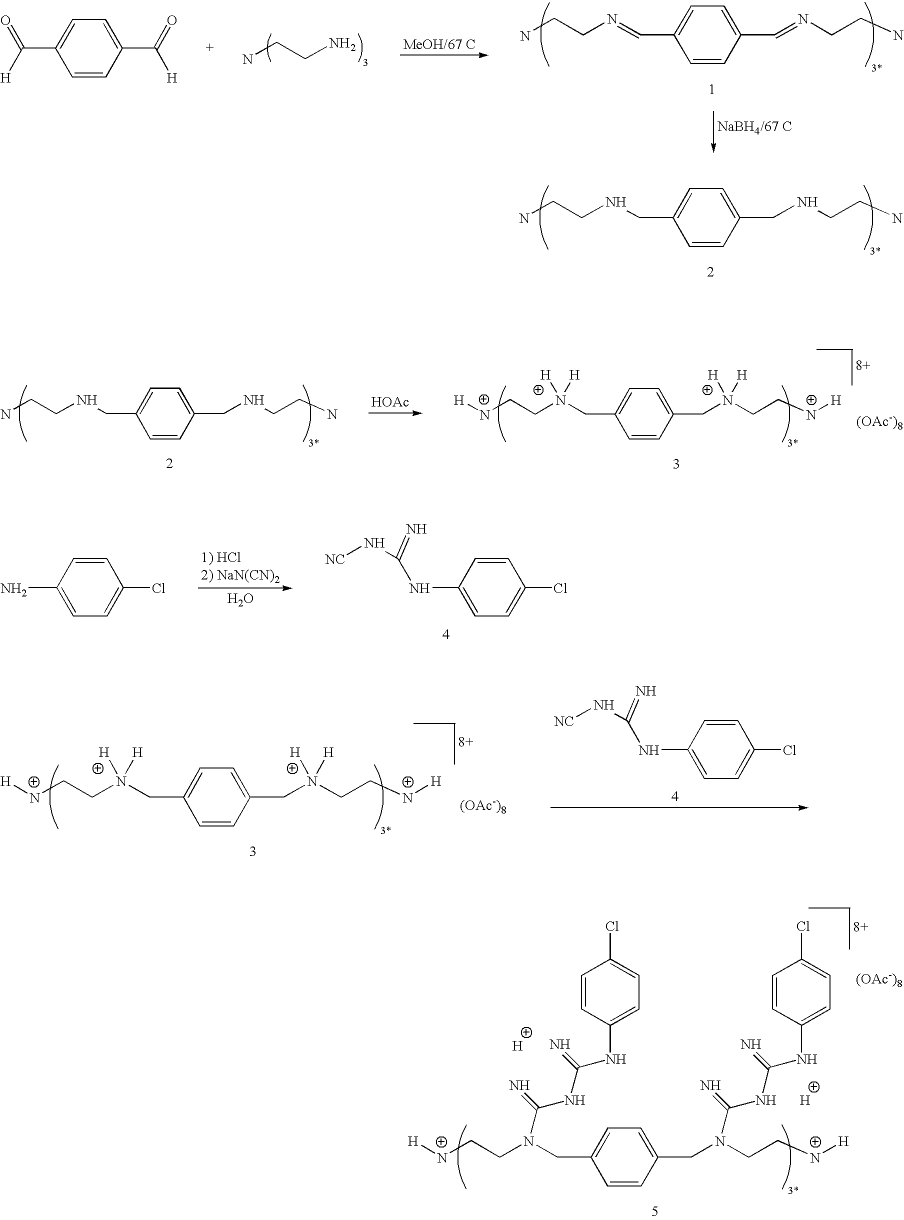

In some embodiments, it may be advantageous to increase the number of active sites on to which to couple pharmaceutically active agents such as depicted directly below in the following two schemes.

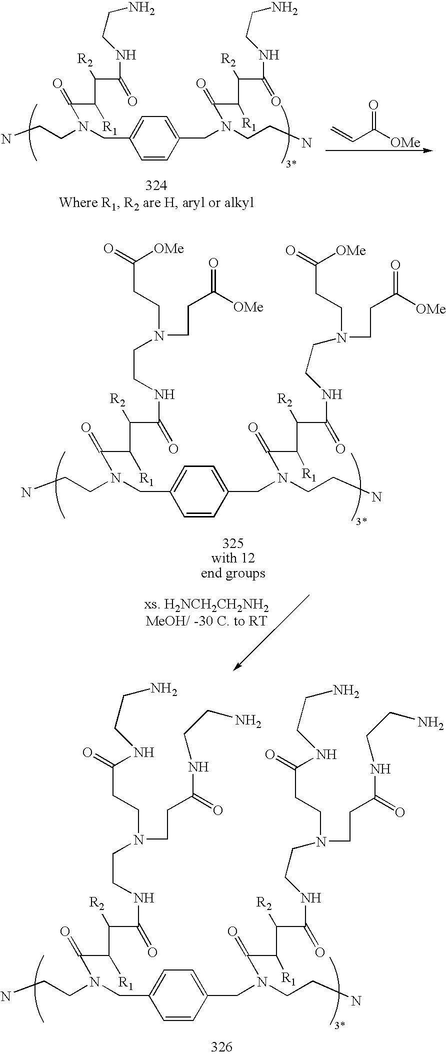

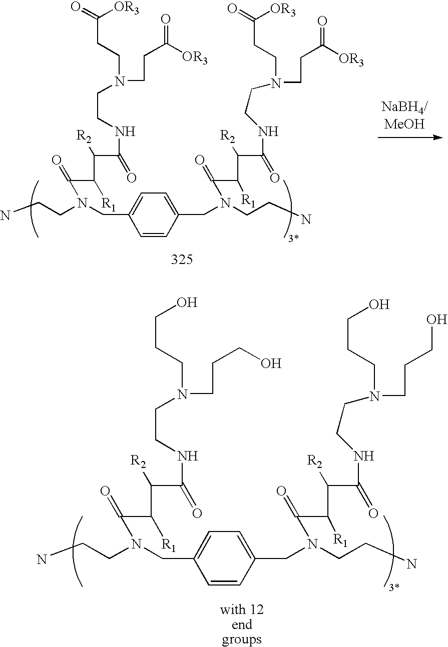

Following are some representative examples of pharmaceutically reactive agents and how to synthetically couple them to linking agents and/or bridged polycyclic compounds such as compound 301.

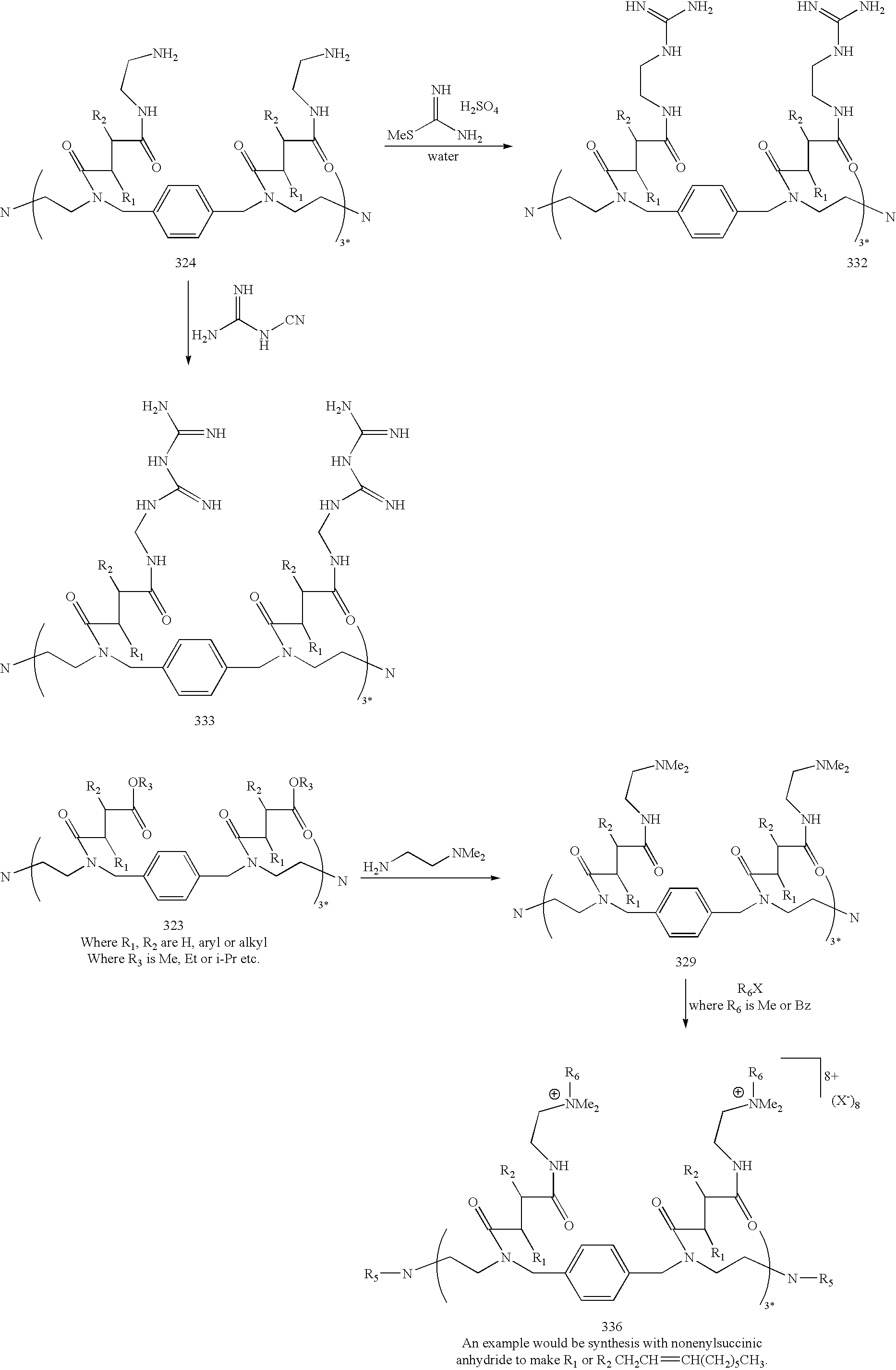

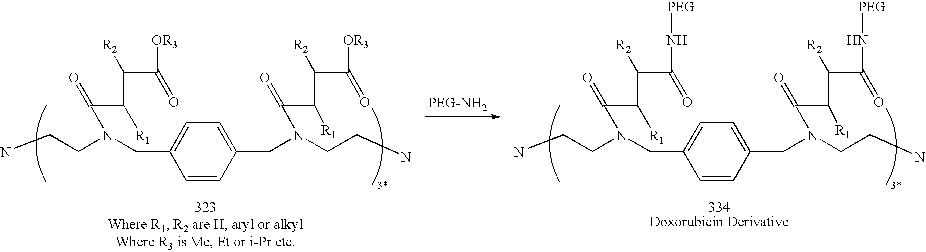

Following are some representative examples of pharmaceutically reactive agents and how to synthetically couple them to linking agents and/or bridged polycyclic compounds such as compound 301. Specifically the following is an example of how to synthesize a slow release nicotinic acid using any lactone or triethylene glycol monochlorohydrin as precursors to the alchohol. Discussions concerning examples of methods of how to perform reactions detailed below are found in U.S. Pat. No. 5,386,018 to Au et al., U.S. Pat. No. 5,212,318 to Buckland et al., and U.S. Pat. No. Re. 32,581 to Scherm et al., which are incorporated by reference as if fully set forth herein.

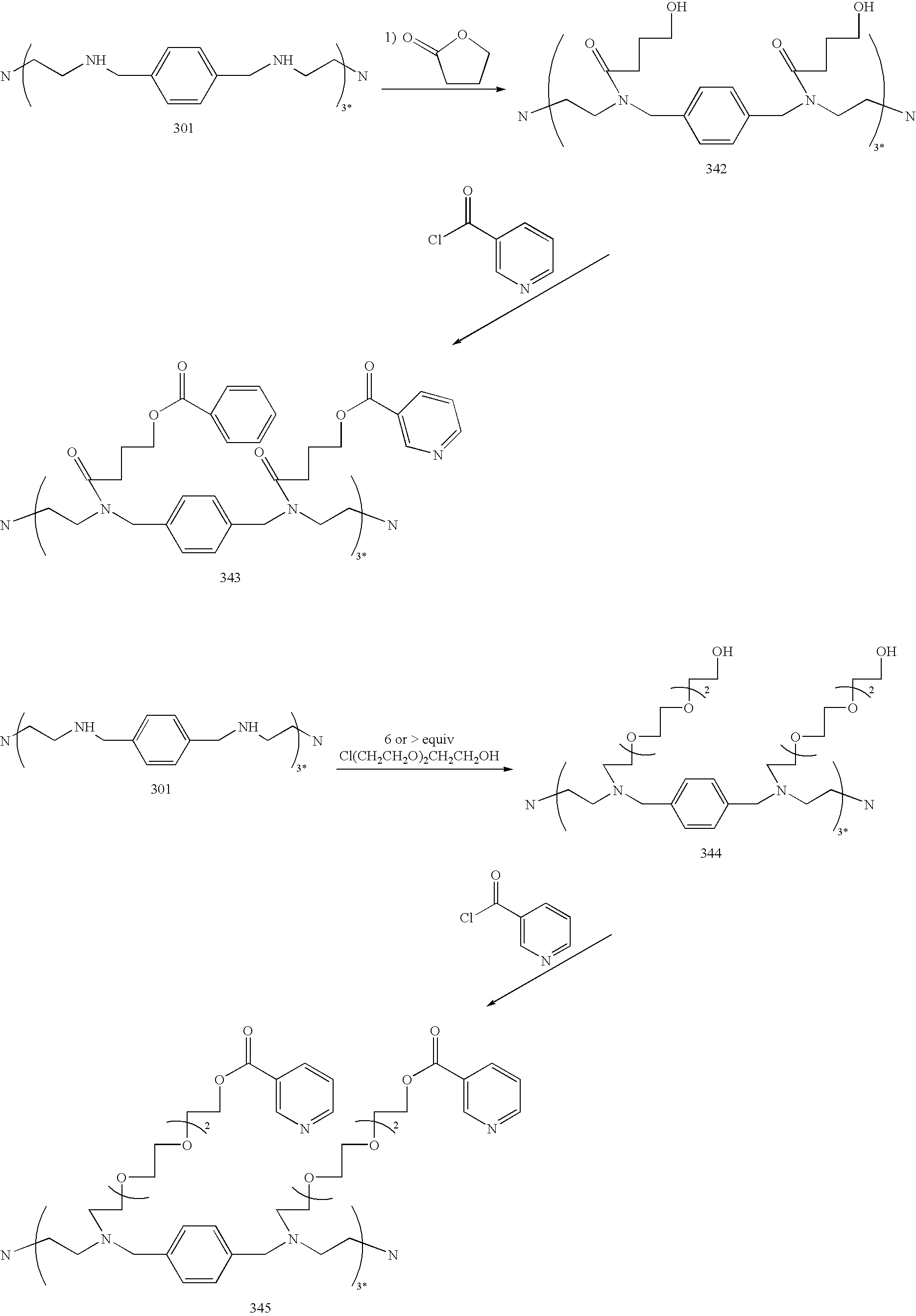

### Dosage and Administration

In some embodiments, bridged polycyclic compounds may be administered at a dosage level up to conventional dosage levels, but will typically be less than about 2 gm per day. Suitable dosage levels may depend upon the overall systemic effect of the chosen pharmaceutical agent coupled to the bridged polycyclic compound, but typically suitable levels will be about 0.001 to 50 mg/kg body weight of the patient per day, from about 0.005 to 30 mg/kg per day, or from about 0.05 to 10 mg/kg per day. The compound may be administered on a regimen of up to 6 times per day, between about 1 to 4 times per day, or once per day.

In some embodiments, bridged polycyclic compounds may be administered at a dosage level of about 2 gm to about 20 gm per day or about 2 gm to 7 gm per day. For further discussions concerning dosage levels for sequestrants can be found in U.S. Pat. No. 7,385,012 to Chang et al. and U.S. Pat. No. 7,342,083 to Chang et al., which are incorporated by reference as if fully set forth herein.

In the case where an oral composition is employed, a suitable dosage range is, e.g. from about 0.01 mg to about 100 mg per kg of body weight per day, preferably from about 0.1 mg to about 10 mg per kg and for cytoprotective use from 0.1 mg to about 100 mg per kg of body weight per day.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

While individual subject needs vary, determination of optimal ranges of effective amounts of each component is with the skill of the art. Typically, a bridged polycyclic compound may be administered to mammals, in particular humans, orally at a dose of 5 to 100 mg per day referenced to the body weight of the mammal or human being treated for a particular disease. Typically, a bridged polycyclic compound may be administered to mammals, in particular humans, parenterally at a dose of between 5 to 1000 mg per day referenced to the body weight of the mammal or human being treated for a particular disease. In other embodiments, about 100 mg of a bridged polycyclic compound is either orally or parenterally administered to treat or prevent disease.

The unit oral dose may comprise from about 0.25 mg to about 1.0 gram, or about 5 to 25 mg. The unit parenteral dose may include from about 25 mg to 1.0 gram, or between 25 mg and 500 mg. polymerized bridged polycyclic compounds may include larger dosages from 1000 to 5000 mg or more as seen with products such as colestipol for example. The unit intracoronary dose may include from about 25 mg to 1.0 gram, or between 25 mg and 100 mg. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a bridged polycyclic compound as a raw chemical, the compounds may be administered as part of a pharmaceutical preparation containing suitable pharmaceutically acceptable carriers, preservatives, excipients and auxiliaries which facilitate processing of the bridged polycyclic compound which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

General guidance in determining effective dose ranges for pharmacologically active compounds and compositions for use in the presently described embodiments may be found, for example, in the publications of the International Conference on Harmonisation and in REMINGTON'S PHARMACEUTICAL SCIENCES, 8^{th} Edition Ed. Bertram G. Katzung, chapters 27 and 28, pp. 484-528 (Mack Publishing Company 1990) and yet further in BASIC & CLINICAL PHARMACOLOGY, chapters 5 and 66, (Lange Medical Books/McGraw-Hill, New York, 2001).

### Pharmaceutical Compositions

Any suitable route of administration may be employed for providing a subject with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulisers. The compounds may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, gels, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Liposomal formulations, in which mixtures of the bridged polycyclic compound with, for example, egg yolk phosphotidylcholine (E-PC), may be made for injection. Optionally, the suspension may contain stabilizers, for example, antioxidants such as BHT, and/or preservatives, such as benzyl alcohol.

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the subject, and the effect desired. A physician or veterinarian can determine and prescribe the effective amount of the drug required.

By way of general guidance, the daily oral dosage of each active ingredient, when used for the indicated effects, will range between about 0.001 to 1000 mg/kg of body weight, between about 0.01 to 100 mg/kg of body weight per day, or between about 1.0 to 20 mg/kg/day. Intravenously administered doses may range from about 1 to about 10 mg/kg/minute during a constant rate infusion. Compounds of this invention may be administered in a single daily dose, or the total daily dosage may be administered in divided doses of two, three, or four or more times daily.

The pharmaceutical compositions described herein may further be administered in intranasal form via topical use of suitable intranasal vehicles, or via transdennal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

In some embodiments, dosage forms (pharmaceutical compositions) suitable for administration may contain from about 1 milligram to about 100 milligrams or more of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase subject acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

In some embodiments, bridged polycyclic compounds may be incorporated into a composition which is substantially nontoxic to an animal and/or human. A composition may include a solvent capable of dissolving a bridged polycyclic compound. In some embodiments, a composition may include an environmentally green solvent. A solvent may include water and/or an alcohol (e.g., ethanol, methanol). In some embodiments, a composition may consist of water and a bridged polycyclic compound (e.g., 308, 309, 312 or gluconate salt of 312). Such compositions may be administered using any method described herein including, but not limited to, orally, topically, intravenously, absorbed through the skin, injected, etc.

In some embodiments, an oral composition may include a flavoring. A flavoring may include something an animal may find palatable. For example a flavoring may include malt extract, xylitol, splenda, sucralose or any sweetener. A flavoring may range from 0.01% to 0.10%, 0.10% to 1.0%, or 1.0% to 10.0% of a composition.

In some embodiments, a composition may include a colorant. A colorant may include D&C Blue #1 or any FDA approved color. A colorant may range from 0.001% to 0.010%, 0.010% to 0.10%, or 0.10% to 1.0% of a composition.

In some embodiments, an oral composition may include a fragrance.

In some embodiments, a composition may include additional additives which may function in combination or separately from the bridged polycyclic compound in solution. Additives may function to improve a subject's health. Additives may include vitamins including, but not limited to, vitamins D and E.

### Compositions Comprising Bridged Polycyclic Compounds

In some embodiments, bridged polycyclic compounds may be incorporated into a composition which is substantially nontoxic to an animal and/or human. A composition may include a solvent capable of dissolving a bridged polycyclic compound. In some embodiments, a composition may include an environmentally green solvent. A solvent may include water and/or ethyl alcohol. In some embodiments, a composition may consist of water and a bridged polycyclic compound. Such compositions may be administered using any method described herein including, but not limited to, orally, topically, intravenously, absorbed through the skin, injected, etc.

In some embodiments, different compositions may be formulated for different types of users. For professionals users (e.g., doctors, veterinaries), compositions may include a greater percentage of active bridged polycyclic compounds than compositions formulated for over the counter sale to nonprofessionals. Professional compositions may not include flavorings or colorants.

In some embodiments, a pharmaceutical composition may include bridged polycyclic compounds. At least one of the bridged polycyclic compounds may include at least two cyclic groups. At least two of the cyclic groups may include quaternary ammonium or amine moieties. In some embodiments at least two of the cyclic groups may be defined at least in part by quaternary ammonium moieties. Bridged polycyclic compounds may include any of the compounds described herein.

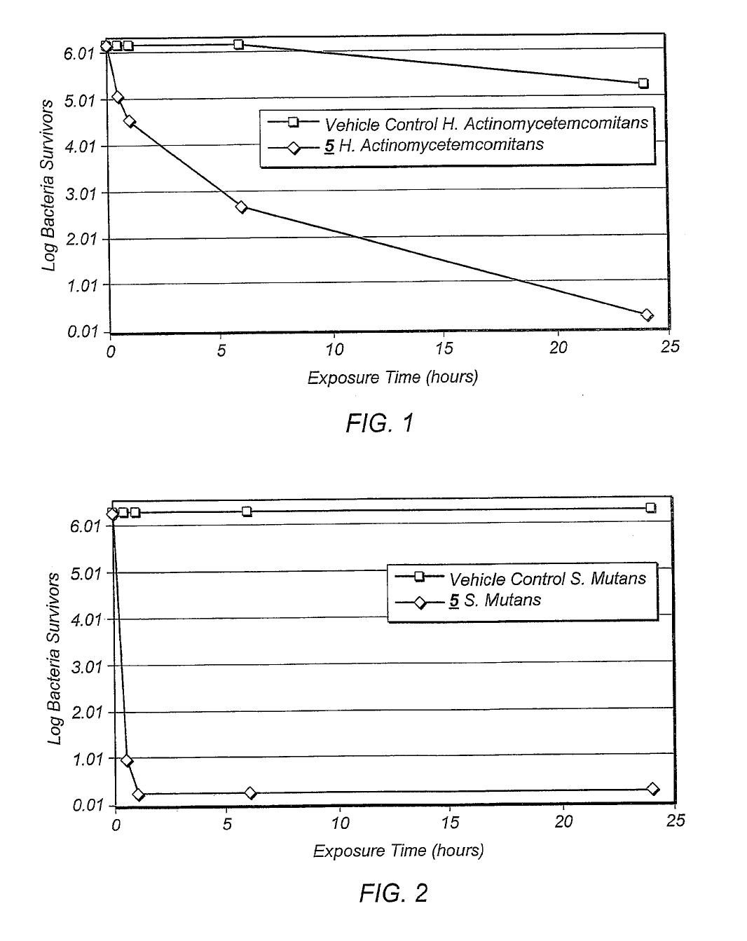

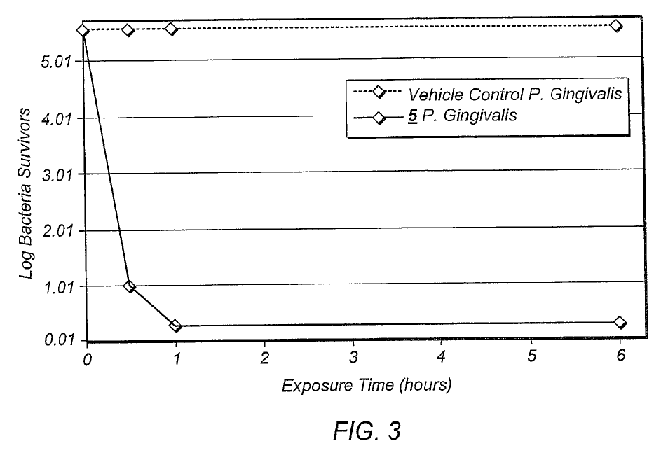

FIGS. 1-3 depict a graphical representation of time kill assay tests for bridged polycyclic compound 5 tested against Haemophilus Actinomycetemcomitans, Streptococcus mutans, and Porphymonas Gingivalis respectively. The test results demonstrate how effective bridged polycyclic compounds are against known destructive microbes.

In some embodiments, pharmaceutical compositions may enhance sustained antimicrobial activity with minimum harm to the living structure and surrounding tissues and without affecting the composition's restorative properties.

### EXAMPLES

Having now described the invention, the same will be more readily understood through reference to the following example(s), which are provided by way of illustration, and are not intended to be limiting of the present invention.

General Experimental: All manipulations were carried out using Schlenk technique. Concentrated hydrochloric acid and acetic acid were purchased from J.T. Baker and used as received. Sodium hydroxide was purchased from Mallinckrodt and used as received. Sodium dicyanamide and sodium bicarbonate were purchased from Aldrich and used as received. Tris(2-aminoethyl)amine was purchased from Acros Organics and distilled before use. Terephthaldicarboxaldehyde and p-chloroaniline were purchased from Aldrich and sublimed before use. Sodium sulfate was purchased from EMD and used as received. Water was sparged for >10 minutes before use. Dichloromethane, ethyl acetate and hexanes were purchased from EMD and used as received. Ethyl alcohol, anhydrous 200 proof, was purchased from Aldrich and used as received. Silica gel 60 (230-400 mesh) was purchased from EMD and used as received. MS analysis was performed on an Applied Biosystems Voyager DE instrument at HT Laboratories in San Diego, Calif. NMR analysis was performed on a JEOL Eclipse⁺ 400 instrument at Acorn NMR, Inc. in Livermore, Calif.

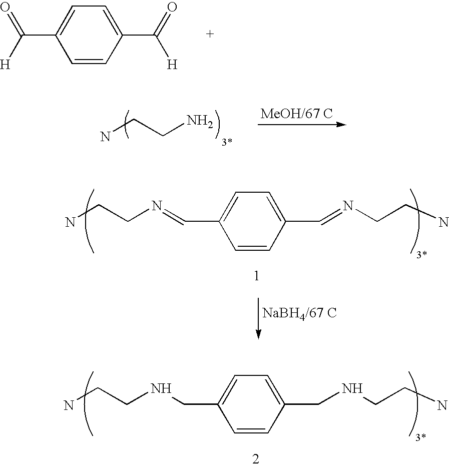

Synthesis of 2: To a 12 L round bottom flask equipped with a reflux condenser and addition funnel was added methanol (8 L) followed by terephthaldicarboxaldehyde (64.4 g, 0.480 moles). The solution was heated to 65° C. and tris(2-aminoethyl)amine (46.8 g, 47.9 mL, 0.320 moles) was added. Then the solution was refluxed for about 16 h and cooled to room temperature. The solution was filtered to another 12 L round bottom flask equipped with a reflux condenser and sodium borohydride (60.5 g, 1.60 moles) was added. The solution was refluxed for about 16 h and cooled to room temperature. The volatiles were removed by rotational evaporator and the residue dissolved in dichloromethane (720 mL) and hydrochloric acid, 1.0 M (3.2 L). It was stirred for 5 minutes. Then to the solution was added sodium hydroxide, 3.0 M (1.6 L), the solution stirred for 5 minutes and the phases separated. The aqueous was extracted with dichloromethane (2×400 mL, 2×200 mL), the organic phase combined, washed with water (2×600 mL) and dried over sodium sulfate. Then the volatiles were removed by vacuum transfer to leave a slightly off white powder (89.6 g, 150 mmoles, 93.5% yield). Analysis of 2: ¹H NMR (400 MHz, CD₂Cl₂, δ): 2.61, 2.76 (m, 24H, NCH₂CH₂NHCH₂C₆H₄), 3.62 (s, 12H, NCH₂CH₂NHCH₂C₆H₄), 6.84 (s, 12H, NCH₂CH₂NHCH₂C₆H₄). ESI-MS (m/z): [M+H]+599, [M+H]²⁺ 300.

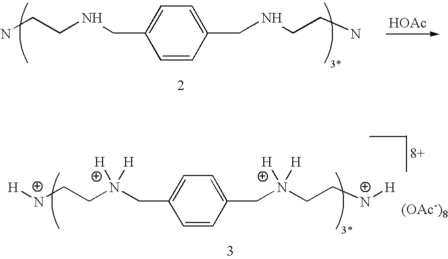

Synthesis of 3: Octa-amine 2 (19.9 g, 33.3 mmoles) was added to a 2 L flask and combined with ethyl acetate (924 mL) and acetic acid (38.1 mL, 40.0 g, 666 mmoles). The solution was filtered and hexanes (629 mL) was added which caused the product to crystallize. The solution was filtered and the precipitate washed with 80% hexanes, 20% ethyl acetate (1500 mL). The product was transferred to a flask and the volatiles removed by vacuum transfer. The supernatant was combined with hexanes (300 mL), filtered and washed with of 80% hexanes, 20% ethyl acetate (1500 mL). The precipitate was transferred to a flask and the volatiles removed by vacuum transfer. To the supernatant was added the wash solution from the second crop which precipitated the third crop of product. The solution was filtered and washed with 80% hexanes, 20% ethyl acetate (1500 mL). The precipitate was transferred to a flask and the volatiles removed by vacuum transfer. The product is a slightly off white powder (33.7 g, 31.3 mmoles, 93.9% yield). Alternatively, 2 is suspended in ethyl alcohol followed by the slow addition of 20 equiv of glacial acetic acid followed by stirring; the solvent and excess acid are removed via rotary evaporation and or a schenk line under vacuum resulting in 3. Analysis of 3: ¹H NMR (400 MHz, Methanol-d₄, δ): 1.88 (s, 24H, CH₃CO₂), 2.78, 3.24 (m, 24H, CH₂CH₂), 4.14 (s, 12H, NCH₂Ph), 7.47 (s, 12H, Ph). MALDI-MS (m/z): [M]⁺ 600, [M+Na]⁺ 622.

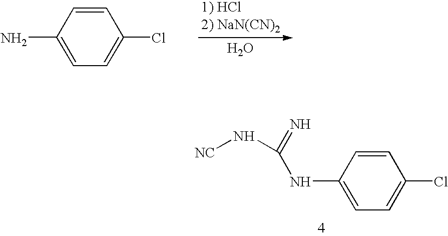

Synthesis of 4: The compound p-chloroaniline (170 g, 1.33 moles) was added to a 1 L flask and dissolved in water (625 mL) and concentrated HCl (111 mL, 1.33 moles). Then in a separate 5 L flask sodium dicyanamide (237 g, 2.66 moles) was dissolved in water (2035 mL) and heated to 50° C. The solution of p-chloroaniline was added to the solution of sodium dicyanamide over 120 minutes, the flask was fitted with a reflux condenser and then the reaction solution was heated for about 16 h at 90° C. Then the reaction solution was allowed to cool and saturated sodium bicarbonate (1500 mL) was added and the solution stirred for 15 minutes. Ethyl acetate (1000 mL) was added and the solution stirred for 10 minutes before the phases were separated. The aqueous phase was extracted with ethyl acetate (10×1000 mL, 500 mL), the organic was combined and washed with saturated brine (3×1200 mL), dried over sodium sulfate (anhydrous) and filtered. A 10 cm deep silica plug was packed with silica/ethyl acetate slurry and then washed with ethyl acetate (2000 mL). The product was sent through the silica plug and the plug washed with ethyl acetate (6000 mL). The volatiles were removed from the filtrate by vacuum transfer until about 10% of the solution remained and the solution was filtered. The product was dried under vacuum to p<20 mtorr to leave a white powder. Then the product was placed under vacuum again at p<20 mtorr while on a 70° C. oil bath for 18 h (203 g, 1.04 moles, 78.3% yield). Analysis of 4: ¹H NMR (400 MHz, DMSO-d₆, δ): 7.08 (s, 2H, PhNHC(NH)NHCN), 7.36 (m, 4H, Ph), 9.15 (s, 1H, PhNHC(NH)NHCN). MALDI-MS (m/z): [M]⁺ 195, [M+Na]⁺ 218.

Synthesis of compound 4 has been described in patent GB599722 to Kenny et al.; Curd, F. H. S. et al. “Synthetic Antimalarials, Part X, Some Aryldiguanide (-biguanide) Derivatives” J. Chem. Soc. 729-737 (1946); and Curd, F. H. S. et al. “Synthetic Antimalarials, Part XXVIII, An alternative route to N1-aryl-N5-alkyldiguanides” J. Chem. Soc. 1630-1636 (1948), which are incorporated by reference as if fully set forth herein. Synthesis of compounds similar to compound 4 are described in U.S. Pat. No. 2,455,807 to Redmon et al. and U.S. Pat. No. 5,534,565 to Zupancic et al., which are incorporated by reference as if fully set forth herein.

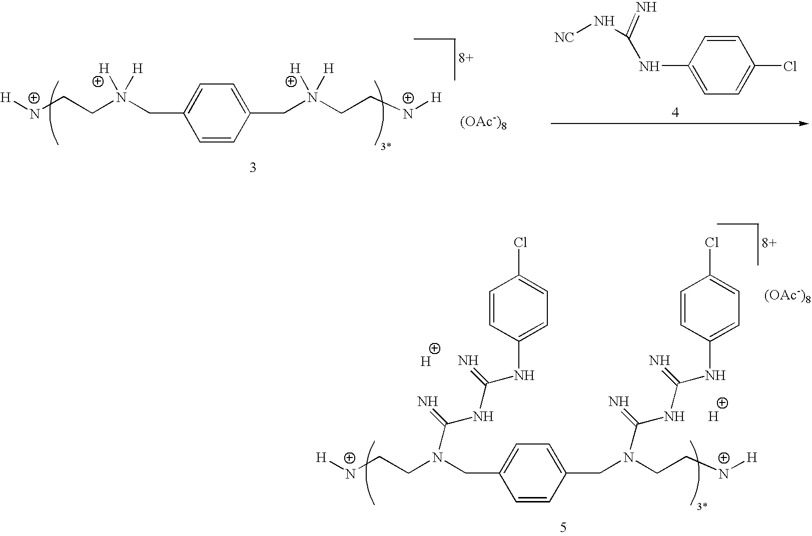

Synthesis of 5: Intermediate 3 (32.9 g, 30.5 mmoles) was added to a 500 mL flask followed by 1-butanol (30.3 mL) which formed a slurry. Then 4 (39.1 g, 201 mmoles) was added. The flask was fitted with a reflux condenser and placed into an oil bath set to reach 90° C. It was heated for 3 days and allowed to cool to room temperature. The volatiles were removed by vacuum transfer and the resulting foam was crushed to a powder. The crude product was dissolved in ethyl alcohol (31.9 mL) and ethyl acetate (65.4 mL). The product was precipitated with ethyl acetate (915 mL) and the solution filtered. Then the product was washed with ethyl acetate (980 mL) and the volatiles removed by vacuum transfer to produce a white powder (57.9 g, 25.8 mmoles, 84.5% yield). Alternatively, the resulting crude product 5 foam was crushed to a powder and dissolved in water followed by extraction of the aqueous phase 3 times with ethyl acetate resulting in an off white powder 5 in ˜77% yield. Analysis of 5: MALDI-MS (m/z): 1269 [M+5 DHB]²⁺, 1423 [M+7 DHB]²⁺ (DHB is MALDI matrix dihydroxybenzoic acid).

The product 5 can be converted to the freebase and then protonated with mineral, organic or other acids to afford the desired counter ion (anion) (for example 5 can be treated with base, isolated as the freebase and treated with acetic acid to regenerate 5, i.e. replace acetic acid with a different acid such as D-Gluconic Acid (or combination of acids) to generate the salt containing the desired anion counter-ion). Analysis of Freebase 5: MALDI-MS (m/z): 883 [M]²⁺

The product 5 can be converted to the freebase and then protonated with mineral, organic or other acids to afford the desired counter ion (anion) (for example 5 can be treated with base, isolated as the freebase and treated with acetic acid to regenerate 5, i.e. replace acetic acid with a different acid such as D-Gluconic Acid, Butyric Acid, Statin Acids (e.g., pravastatin, fluvastatin, atorvastatin, etc.), Nicotinic Acid (i.e. Niacin), (or combination of acids) to generate the salt containing the desired anion counter-ion and/or mixture).

Combining of the Freebase of 5 and Nicotinic Acid: To a vial and stirbar, (0.038 mg, 0.0215 mmol) of the freebase of 5 was added followed by 0.76 mL of deionized water, 0.620 of ethyl alchohol and 8 equiv. (0.017 g, 0.14 mmol) of Nicotinic acid (also known as Niacin or vitamin B₃). The mixture was stirred for about 1 h resulting in a clear homogeneous solution. MALDI-MS (m/z): 1379 [M]²⁺[M]²⁺ −1 Nicotinic Acid 1314; [M]²⁺ −2 Nicotinic Acid 1253

Combining of the Freebase of 5 and Nalidixic Acid: To a vial and stirbar, (0.026 mg, 0.015 mmol) of the freebase of 5 was added followed by 2.0 mL of acetone, 1.0 mL of deionized water and 8 equiv. (0.028 g, 0.12 mmol) of Nalidixic acid. The mixture was stirred for about 1 h to form a clear homogeneous solution. MALDI-MS (m/z): 1210 [M] ³⁺ [M]²⁺ −1 Nalidixic Acid 1697; [M]²⁺ −2 Nalidixic Acid 1580.

Combining of the Freebase of 5 and Butyric Acid: To a vial and stirbar, (0.027 mg, 0.015 mmol) of the freebase of 5 was added followed by 2.0 mL of acetone, 1.0 mL of deionized water and 8 equiv. (0.011 mL, 0.12 mmol) of Butyric acid. The mixture was stirred for about 1 h to form a clear homogeneous solution.

Formulation of Topical Sealant

General: Poly(vinyl acetate-co-crotonic acid) beads and ethyl alcohol (anhydrous 200 proof) were purchased from Aldrich and used without further purification.

Example: Poly(vinyl acetate-co-crotonic acid) (0.80 g) was dissolved in water (1.74 mL) and ethyl alcohol (7.81 mL). The active ingredient 5 (0.50 g) was dissolved in ethyl alcohol (1.00 mL). The two solutions were combined and mixed for 2 hours.

Formulation of Topical Composition

Example: The active ingredient 5 was dissolved in ethyl alcohol and/or water.

Other common topical formulation or coating formulation components known by those skilled in the art may be used in conjunction with or in place of the above example.

### General Experimental Procedures for Animal Studies:

Test Article Palatability Study: Due to the dietary route of exposure of test article a palatability study is conducted prior to the efficacy study of test article dosing. The palatability study is commonly conducted to determine what level of test article can be mixed with food and be accepted by the test subject. The palatability study is short in duration (days) and an appropriate sweetener or other palatable additive is mixed at varied levels with the food and test article. The percentage of required sweetener additive is determined to meet the desired highest level of test article dosing. When the palatability study shows a limit below the desired highest level of dosing, dosing parameters are adjusted accordingly.

Justification of Test System and Number of Animals: The Sprague Dawley rat is chosen as the animal model for this study as it is a preferred rodent species for preclinical toxicity testing by regulatory agencies.

The total number of animals to be used in this type of study is the minimum required to properly characterize the effects of the test article. The number of groups is based on guidelines of the Modification of OECD Testing Guideline No. 4071. Based on statistical sample size calculations 2, the number of animals per group is the minimum necessary to detect a 19% difference in body weight as statistically significant when compared to a mean body weight of 250 g with a standard deviation of 20 g at an α level of 0.05 and a β level of 0.1. From a toxicological perspective, this degree of statistical resolution is considered sufficient for this study design.

Justification of Route and Dose Levels: The dietary route of exposure is selected since this is the intended route of animal exposure. The dose levels are selected based on an acute oral gavage study in rats was conducted with test article and the resulting LD50. Therefore, in a feed study the total exposure should be less than the acute study and any toxic effect should be minimized.

Preparation of Dose Formulations: Test article dosing formulations are prepared at appropriate concentrations (w/w) to meet dosage level requirements. The test diet (e.g., rodent meal) will be formulated to contain the appropriate amount of test article. The required amount of test article will be added initially to a small amount of rodent meal and mixed (e.g., in a Kitchen Aid mixer) for an approximate amount of time (to be documented). The resulting premix will then be added to the final mix in an 8 kg Hobart blender or appropriately sized mixer (if a Kitchen Aid mixer is used for final diet preparation, then a premix preparation will not be necessary) and blended for an appropriate amount of time (to be documented). Any additional mixing details will be documented in the raw data. The test diet will be prepared approximately weekly in order for new test diet to be offered on Days 1 and 7. Animals will receive the test diet ad libitum for the extent of the in-life portion of the study. The prepared test diet will remain at room temperature during the dosing period.

Food: PMI Nutrition International Certified Rodent Chow® #5002 meal will be provided ad libitum throughout the study, except during fasting for clinical pathology determinations. The lot number and expiration date of each batch of diet used during the study will be recorded. The feed is analyzed by the supplier for nutritional components and environmental contaminants. Results of the dietary analyses are provided by the manufacturer for each lot of diet and are on file at the Testing Facility.

Water: Municipal tap water following treatment by reverse osmosis and ultraviolet irradiation will be available ad libitum to each animal via an automatic watering system. The water may also be provided to individual animals via water bottles during urinalysis collection or as warranted by clinical signs or other health changes. The water is analyzed semi-annually for microbial contamination and for total dissolved solids, hardness, and various environmental contaminants. Results of these analyses are on file at the Testing Facility. Based on the results of the most recent analyses, there are no contaminants in the water that would interfere with the conduct or interpretation of the study.

Experimental Design: The experimental design is shown in the following table:

### Experimental Design for the Toxicity and Toxicokinetic Phases

| | | | | Dose |
|---|---|---|---|---|
| | | | Target | Concentration |
| | No. of | Dose | Dose Level | (mg/kg |
| Group No. | Males | Material | (mg/kg/day) | diet)^{a} (PPM) |
| 1 | 5 | Control | 0 | 0 |
| | | Article | | |
| 2 | 5 | Test Article | 100 | 1027 |
| 3 | 5 | Test Article | 500 | 5135 |
| 4 | 5 | Test Article | 1000 | 10269 |

PPM = parts per million
^{a}Dose concentrations will be based on a 267-gram male rat consuming 26 grams of test diet/day.

Administration of Test/Control Articles: Control diet (Group 1) or test article-treated diet (Groups 2-4) will be administered ad libitum to the appropriate animals from Days 1-14. New control and test article-treated diet will be provided on Days 1 and 7. The test article in the diet will be maintained at a constant concentration (%) for each group. Mean test article consumption for each sex/group will be calculated weekly. The first day of dosing will be designated as Study Day 1.

Body Weights: Frequency: Days −1, 1, 3, 6, 9, 12, and 14. Procedure: Each animal will be weighed on the days specified above. Terminal body weights will not be collected from animals found dead or euthanized moribund.

Food Consumption: Frequency: Days 1-3, 3-6, 7-9, 9-12, and 12-14. Procedure: Food consumption will be quantitatively measured on each day. The animals will be fasted overnight prior to clinical chemistry blood collection on Days 7 and 15.

### Clinical Pathology

Sample Collection: Animals will be fasted overnight prior to scheduled clinical pathology sample collections, but will have access to water ad libitum. Samples will be collected according to the following table:

Samples for Clinical Pathology Evaluation

| Group Nos. | Time Point | Clinical Chemistry |
|---|---|---|
| 1-4 | Day 7 | x |
| 1-4 | Day 15 | x |

Note:
“x” = sample to be collected.

### Clinical Chemistry

Method of Collection: Jugular vein (under isoflurane anesthesia). [The orbital plexus (under isoflurane anesthesia) may be used if blood can not be obtained via the jugular vein.]

Target Volume: Day 7-1 mL/sample
Day 15-2 mL/sample

Anticoagulant: None

Processing: To serum

Clinical Chemistry Parameters

| | |
|---|---|
| Alanine aminotransferase (ALT) | Total protein |
| Aspartate aminotransferase (AST) | Albumin |
| Alkaline phosphatase (Alk Phos'tase) | Globulin |
| Gamma-glutamyltransferase (GGT) | Albumin/globulin ratio (A/G |
| Total bilirubin | Ratio) |
| Urea nitrogen | Glucose |
| Creatinine | Cholesterol |
| Calcium | Triglycerides |
| Phosphorus | Sodium |
| Chloride | Potassium |

### Statistical Analysis

Inferential statistical analyses will be performed using the Compaq Alpha DS10 Computer. Each data set collected for the toxicity phase animals will be subjected to a statistical decision tree. At a minimum, the following parameters and endpoints will be analyzed: body weights, body weight changes, food consumption, and clinical chemistry.

Data sets for each interval will be initially analyzed for homogeneity of variance using Levene's test4 followed by the Shapiro-Wilk test5 for normality. A p<0.001 level of significance will be required for each test to reject the null hypothesis.

If both Levene's test and the Shapiro-Wilk test are not significant, a single-factor parametric ANOVA6 will be applied, with animal grouping as the factor, using a p<0.05 level of significance. If the parametric ANOVA is significant at p<0.05, Dunnett's test will be used to identify statistically significant differences between the control group and each test article-treated group using a minimum significance level of p<0.05.

If either Levene's test and/or the Shapiro-Wilk test are significant, then the Kruskal-Wallis non-parametric ANOVA7 will be applied, with animal grouping as the factor, using a p<0.05 level of significance. If the non-parametric Kruskal-Wallis ANOVA is significant at p<0.05, Dunn's test8 will be used to identify statistically significant differences between the control group and each test article-treated group using a minimum significance level of p<0.05.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of controlling cholesterol levels in a subject, comprising:
administering a pharmaceutically effective amount of a composition to a subject, the composition comprising at least one bridged polycyclic compound, wherein the bridged polycyclic compound comprises a general structure (1b): 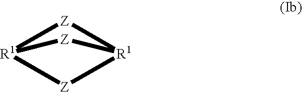
wherein Z comprises at least one bridge, wherein each bridge is independently —R²—N⁺R³₂—R⁴—N⁺R³₂—R², —R²—NR³—R⁴—N⁺R³₂—R²—, —R²—NR³—R⁴—NR³—R²—, or —R²—N═R⁴═N—R²—, and wherein each bridge independently couples R¹ to R¹;
wherein each R¹ is independently N, N⁺H, N⁺R³, a heterocycle group, or a substituted heterocycle group;
wherein each R² is independently an alkyl group, a substituted alkyl group, or an alkene;
wherein each R³ independently comprises a pharmaceutically active agent, an alkyl-aryl group, a substituted alkyl-aryl group, an alkyl group, a substituted alkyl group, an aryl group, a substituted aryl group, a heterocycle group, a substituted heterocycle group, an alkene, an ether, a guanidine, a PEG, a PEI, or a guanidine derivative;
wherein each R⁴ is independently an alkyl-aryl group, a substituted alkyl-aryl group, an aryl group, or a substituted aryl group; and
altering cholesterol levels in the subject.

2. (canceled)

3. (canceled)

4. (canceled)

5. The method of claim 1, further comprising reducing cholesterol levels.

6. The method of claim 1, further comprising reducing triglyceride levels.

7. The method of claim 1, further comprising reducing weight levels.

8. The method of claim 1, further comprising reducing lipid levels.

9. The method of claim 1, further comprising inhibiting LDL production.

10. The method of claim 1, further comprising increasing LDL absorption.

11. The method of claim 1, further comprising increasing hepatic LDL uptake.

12. The method of claim 1, further comprising inhibiting conversion of carbohydrates into fat.

13. The method of claim 1, further comprising inhibiting hunger.

14. The method of claim 1, further comprising inhibiting MTP protein.

15. The method of claim 1, wherein at least one R³ comprises a guanidine or a guanidine derivative.

16. The method of claim 1, wherein at least one R³ comprises a phenol or a phenol derivative.

17. The method of claim 1, wherein at least one R³ comprises a nicotinic acid derivative.

18. The method of claim 1, wherein at least one of the bridged polycyclic compounds is a salt of the bridged polycyclic compounds.

19. The method of claim 1, wherein at least one of the bridged polycyclic compounds is a salt of the bridged polycyclic compounds, and wherein at least one counterion forming the salt is an acetic acid derivative, a pinolenic acid derivative, a nicotinic acid derivative, a hydroxycitric acid derivative, a propionic, acid derivative, a butyric acid derivative, a D-Gluconic acid derivative, a Statin acid derivative, or a short chain alkyl carboxylic acid derivative.

20. The method of claim 19, wherein at least one Statin acid derivative comprises a pravastatin acid derivative, a fluvastatin acid derivative, a atorvastatin acid derivative, a lovastatin acid derivative, or a simvastatin acid derivative.

21. (canceled)

22. (canceled)

23. (canceled)

24. (canceled)

25. The method of claim 1, wherein at least one of the pharmaceutically active agents is a Statin or Statin derivative.

26. The method of claim 1, wherein, the subject is a canine.

27. The method of claim 1 wherein, the subject is a feline.

28. The method of claim 1, wherein the subject is an animal.

29. The method of claim 1, wherein the subject is a human.

30. The method of claim 1, wherein the subject is an avian, a reptile, a horses, a pig, a sheep, a goat, a deer, a tiger, and/or a lion.

31. (canceled)

32. (canceled)

33. (canceled)

34. The method of claim 1, wherein Z comprises at least one bridge, wherein at least one of the bridges is 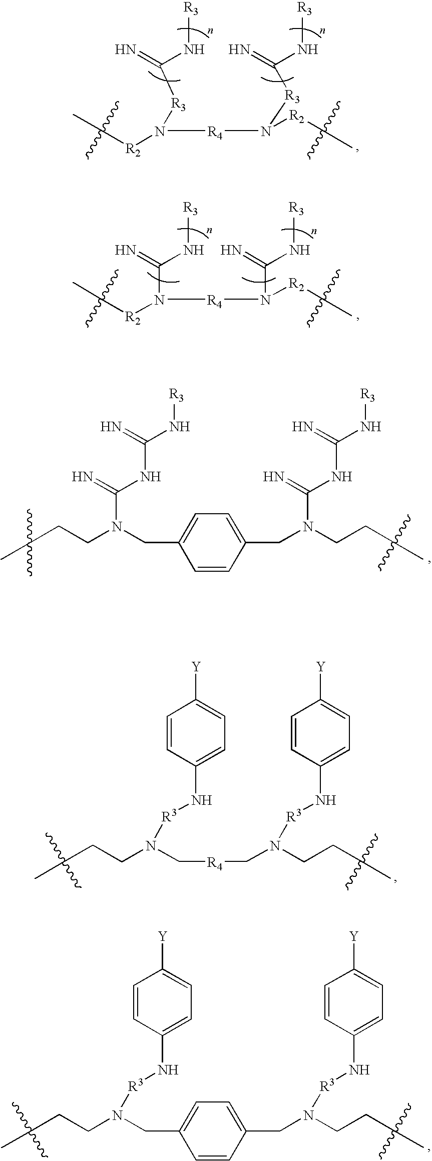 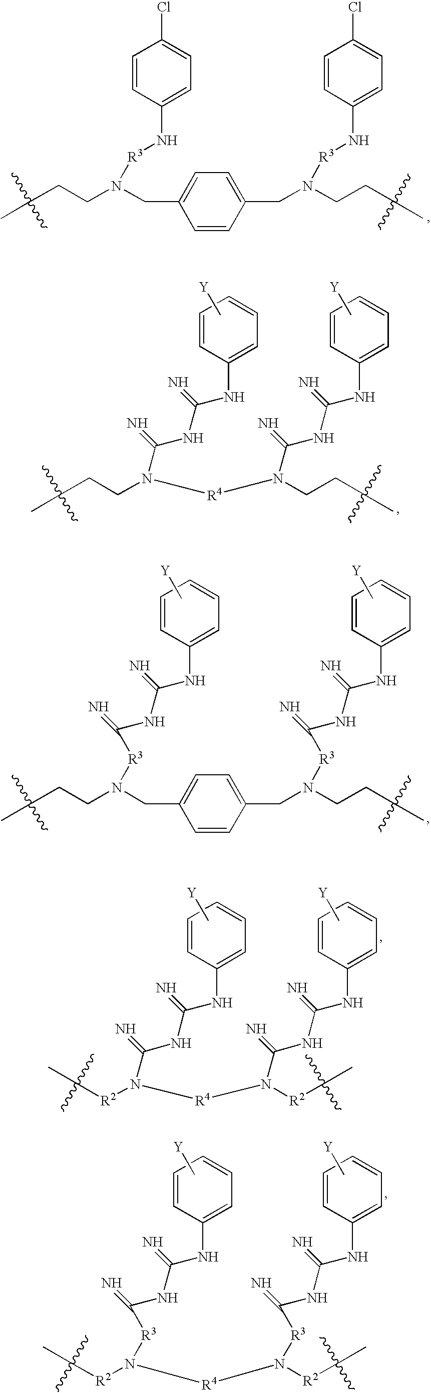 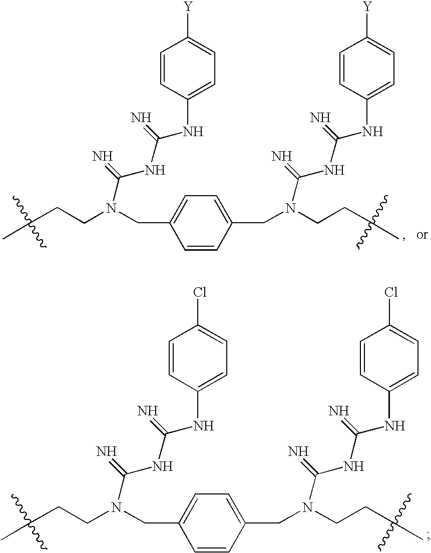
wherein the bridged polycyclic compound is a salt of the bridged polycyclic compound, and wherein at least one counter ion forming the salt is derived from PEG acid, PEG diacid, acetic acid, pinolenic acid, nicotinic acid, hydroxycitric acid, butyric acid, a short chain alkyl carboxylic acid, propionic, acid, D-Gluconic acid, or a Statin acid;
wherein n ranges from 1-10, 2-8, 2-4, 3-6, 2-3, or 1-3; and
wherein Y comprises a halogen, an alcohol, NPEG, OPEG, nicotinic acid derivative or a pharmaceutical active agent.

35. The method of claim 1, wherein Z is 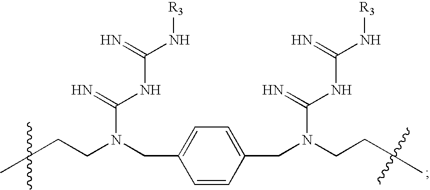
wherein the bridged polycyclic is a salt of the bridged polycyclic compound, and wherein at least one counter ion forming the salt is derived from PEG acid, PEG diacid, acetic acid, pinolenic acid, nicotinic acid, hydroxycitric acid, butyric acid, a short chain alkyl carboxylic acid, propionic, acid, D-Gluconic acid, or a Statin acid.

36. The method of claim 1, wherein Z is 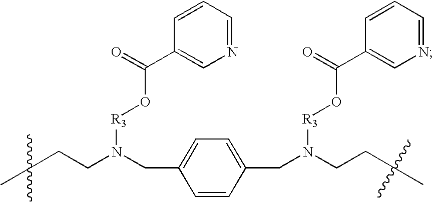
wherein the bridged polycyclic is a salt of the bridged polycyclic compound, and wherein at least one counter ion forming the salt is derived from PEG acid, PEG diacid, acetic acid, pinolenic acid, nicotinic acid, hydroxycitric acid, butyric acid, a short chain alkyl carboxylic acid, propionic, acid, D-Gluconic acid, or a Statin acid.

37. A pharmaceutical composition for altering cholesterol levels, comprising:
a chemical composition comprising at least one bridged polycyclic compound, wherein the bridged polycyclic compound comprises a general structure (1b): 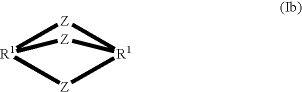
wherein Z comprises at least one bridge, wherein each bridge is independently —R²—N⁺R³₂—R⁴—N⁺R³₂—R²—, —R²—NR³—R⁴—N⁺R³₂—R²—, —R²—NR³—R⁴—NR³—R²—, or —R²—N═R⁴═N—R²—, and wherein each bridge independently couples R¹ to R¹;
wherein each R¹ is independently N, N⁺H, N⁺R³, a heterocycle group, or a substituted heterocycle group;
wherein each R² is independently an alkyl group, a substituted alkyl group, or an alkene;
wherein each R³ independently comprises a pharmaceutically active agent, an alkyl-aryl group, a substituted alkyl-aryl group, an alkyl group, a'substituted alkyl group, an aryl group, a substituted aryl group, a heterocycle group, a substituted heterocycle group, an alkene, an ether, a guanidine, a PEG, a PEI, or a guanidine derivative;
wherein each R⁴ is independently an alkyl-aryl group, a substituted alkyl-aryl group, an aryl group, or a substituted aryl group; and
and wherein at least one of the bridged polycyclic compounds is configured to alter cholesterol levels when administered in pharmaceutically effective amounts to a subject.

38-75. (canceled)

76. A chemical compound, comprising:
a bridged polycyclic compound, wherein the bridged polycyclic compound comprises a general structure (1b): 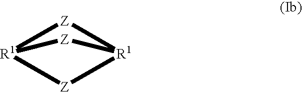
wherein Z comprises at least one bridge, wherein each bridge is independently —R²—N⁺R³₂—R⁴—N⁺R³₂—R²—, —R²—NR³—R⁴—N⁺R³₂—R²—, —R²—NR³—R⁴—NR³—R²—, or —R²—N═R⁴═N—R²—, and wherein each bridge independently couples R¹ to R¹;
wherein each R¹ is independently N, N⁺H, N⁺R³, a heterocycle group, or a substituted heterocycle group;
wherein each R² is independently an alkyl group, a substituted alkyl group, or an alkene;
wherein each R³ independently comprises a pharmaceutically active agent, an alkyl-aryl group, a substituted alkyl-aryl group, an alkyl group, a substituted alkyl group, an aryl group, a substituted aryl group, a heterocycle group, a substituted heterocycle group, an alkene, an ether, a guanidine, a PEG, a PEI, or a guanidine derivative, wherein at least one R³ comprises at least one guanidine, guanidine derivative, or pharmaceutically active agent, and wherein at least one of the pharmaceutically active agents is a Statin or Statin derivative;
wherein each R⁴ is independently an alkyl-aryl group, a substituted alkyl-aryl group, an aryl group, or a substituted aryl group; and
wherein the chemical compound is a salt of the bridged polycyclic compound, and wherein at least one counterion forming the salt is a pinolenic acid derivative, a nicotinic acid derivative, a hydroxycitric acid derivative, a propionic, acid derivative, a butyric acid derivative. a D-Gluconic acid derivative a Statin acid derivative, or a short chain alkyl carboxylic acid derivative.

77-95. (canceled)
